(19) United States
(12) Patent Application Publication
(10) Pub. No.: US 2025/0339200 A1
(43) Pub. Date: Nov. 6, 2025

(54) DEVICES AND METHODS FOR TREATING A LUMEN

(71) Applicant: Boston Scientific Scimed, Inc., Maple Grove, MN (US)

(72) Inventors: Theresa Williams, Savage, MN (US); Tina Marie Lenneman, Otsego, MN (US); Clare Smith, Novi, MI (US)

(73) Assignee: Boston Scientific Scimed, Inc., Maple Grove, MN (US)

(21) Appl. No.: 19/195,986

(22) Filed: May 1, 2025

Related U.S. Application Data
(60) Provisional application No. 63/641,627, filed on May 2, 2024.

(51) Int. Cl. A61B 18/14; A61B 18/00; A61B 90/00

(52) U.S. Cl. CPC A61B 18/1492; A61B 2018/00404; A61B 2018/00577; A61B 2090/0811

(57) ABSTRACT

Medical devices for the heat treatment of a vessel or lumen. An illustrative medical device may comprise a flexible elongate shaft, a first heating element having a first length positioned adjacent to a distal end of the flexible elongate shaft, a second heating element having a second length different from the first length, a first plurality of marking segments extending from a proximal end of the flexible elongate shaft to an intermediate region, each marking segment of the first plurality of marking segments having a third length, and a second plurality of marking segments extending distally from the first plurality of marking segments, each marking segment of the second plurality of marking segments spaced from one another by a fourth length less than the first length. The first heating element may be configured to operate alone or in combination with the second heating element.

### CROSS-REFERENCE TO RELATED APPLICATIONS

This application claims the benefit of priority under 35 U.S.C. § 119 of U.S. Provisional Application No. 63/641,627, filed May 2, 2024, the entire disclosure of which is hereby incorporated by reference.

### TECHNICAL FIELD

The present disclosure pertains to medical devices and methods for manufacturing and/or using medical devices. More particularly, the present disclosure pertains to configurations of a system for closing body lumens.

### BACKGROUND

A wide variety of intracorporeal medical devices have been developed for medical use, for example, surgical and/or intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. There is an ongoing need to provide alternative medical devices as well as alternative methods for manufacturing and/or using medical devices.

### SUMMARY

The disclosure is directed to several alternative designs, materials and methods of manufacturing medical device structures and assemblies.

In a first example, a medical device may comprise a flexible elongate shaft extending from a proximal end to a distal end, a first heating element having a first length (L1) positioned adjacent to the distal end of the flexible elongate shaft, a second heating element having a second length (L2) different from the first length, the second heating element positioned proximal to and abutting a proximal end of the first heating element, a first plurality of marking segments extending from a proximal end of the flexible elongate shaft to an intermediate region positioned between the proximal end and the distal end of the flexible elongate shaft, each marking segment of the first plurality of marking segments having a third length, wherein the third length may satisfy the following equation:

$$L 3 < \frac{L 1 + L 2}{2}$$

where L3 is the third length, L1 is the first length and L2 is the second length, a second plurality of marking segments extending distally from a location distal to the first plurality of marking segments, each marking segment of the second plurality of marking segments spaced from one another by a fourth length, wherein the fourth length may satisfy the following equation:

$$L 4 < L 1$$

where L4 is the fourth length. The first heating element may be configured to operate alone or in combination with the second heating element.

Alternatively or additionally to any of the examples above, in another example, the medical device may further comprise a first sheath removal marking segment. The first sheath removal marking segment may be disposed between the first plurality of marking segments and the second plurality of marking segments.

Alternatively or additionally to any of the examples above, in another example, the medical device may further comprise a second sheath removal marking segment. The second sheath removal marking segment may be positioned proximal to and abutting a proximal end of the second heating element.

Alternatively or additionally to any of the examples above, in another example, the medical device may further comprise a first final treatment indicator. The first final treatment indicator may provide an indication of a final treatment using the first heating element in combination with the second heating element.

Alternatively or additionally to any of the examples above, in another example, the medical device may further comprise a second final treatment indicator. The second final treatment indicator may provide an indication of a final treatment using the first heating element.

Alternatively or additionally to any of the examples above, in another example the second final treatment indicator may be distal to the first final treatment indicator.

Alternatively or additionally to any of the examples above, in another example the first length of the first heating element may be less than the second length of the second heating element.

Alternatively or additionally to any of the examples above, in another example, the third length may satisfy the following equation:

$$L 3 = \frac{( ( L 1 + L 2 ) - OL )}{2}$$

where OL is a treatment overlap length.

Alternatively or additionally to any of the examples above, in another example the fourth length may satisfy the following equation:

$$L 4 = L 1 - O L$$

where OL is a treatment overlap length.

Alternatively or additionally to any of the examples above, in another example the third length may satisfy the following equation:

$$L 3 = L 1 - O L$$

where OL is a treatment overlap length.

In another example, a medical device may comprise a flexible elongate shaft extending from a proximal end to a distal end, a first heating element having a first length (L1) positioned adjacent to the distal end of the flexible elongate shaft, a second heating element having a second length (L2) different from the first length, the second heating element positioned proximal to and abutting a proximal end of the first heating element, a first plurality of marking segments extending from a proximal end of the flexible elongate shaft to an intermediate region positioned between the proximal end and the distal end of the flexible elongate shaft, each marking segment of the first plurality of marking segments having a third length (L3), the third length less than the first length of the first heating element, and a second plurality of marking segments extending distally from a location distal to the first plurality of marking segments, each marking segment of the second plurality of marking segments spaced from one another by a fourth length (L4), the fourth length less than the first length of the first heating element. The first heating element may be configured to operate alone or in combination with the second heating element.

Alternatively or additionally to any of the examples above, in another example, the medical device may further comprise a first sheath removal marking segment. The first sheath removal marking segment may be disposed between the first plurality of marking segments and the second plurality of marking segments.

Alternatively or additionally to any of the examples above, in another example, the medical device may further comprise a second sheath removal marking segment. The second sheath removal marking segment may be positioned proximal to and abutting a proximal end of the second heating element.

Alternatively or additionally to any of the examples above, in another example, the medical device may further comprise a first final treatment indicator. The first final treatment indicator may provide an indication of a final treatment using the first heating element in combination with the second heating element.

Alternatively or additionally to any of the examples above, in another example, the medical device may further comprise a second final treatment indicator. The second final treatment indicator may provide an indication of a final treatment using the first heating element.

Alternatively or additionally to any of the examples above, in another example, the second final treatment indicator may be distal to the first final treatment indicator.

Alternatively or additionally to any of the examples above, in another example, the first length of the first heating element may be less than the second length of the second heating element.

Alternatively or additionally to any of the examples above, in another example, the third length may satisfy the following equation:

$$L 3 = L 1 - O L$$

where L3 is the third length, L1 is the first length, and OL is a treatment overlap length.

Alternatively or additionally to any of the examples above, in another example, the fourth length may satisfy the following equation:

$$L 4 = L 1 - O L$$

where L4 is the fourth length, L1 is the first length, and OL is a treatment overlap length.

In another example, a medical device may comprise a flexible elongate shaft extending from a proximal end to a distal end, a first heating element having a first length positioned adjacent to the distal end of the flexible elongate shaft, a second heating element having a second length different from the first length, the second heating element positioned proximal to and abutting a proximal end of the first heating element, a first plurality of marking segments extending from a proximal end of the flexible elongate shaft to an intermediate region positioned between the proximal end and the distal end of the flexible elongate shaft, each marking segment of the first plurality of marking segments having a third length, and a second plurality of marking segments extending distally from a location distal to the first plurality of marking segments, each marking segment of the second plurality of marking segments spaced from one another by a fourth length, the fourth length less than the first length of the first heating element. The first heating element may be configured to operate alone or in combination with the second heating element.

In another example, a medical device may comprise a flexible elongate shaft extending from a proximal end to a distal end, a first heating element having a first length (L1) positioned adjacent to the distal end of the flexible elongate shaft, a second heating element having a second length (L2) different from the first length, the second heating element positioned proximal to and abutting a proximal end of the first heating element, a first plurality of marking segments extending from a proximal end of the flexible elongate shaft to an intermediate region positioned between the proximal end and the distal end of the flexible elongate shaft, each marking segment of the first plurality of marking segments having a third length, wherein the third length may satisfy the following equation:

$$L 3 < \frac{L 1 + L 2}{2}$$

where L3 is the third length, L1 is the first length and L2 is the second length, a second plurality of marking segments extending distally from a location distal to the first plurality of marking segments, each marking segment of the second plurality of marking segments spaced from one another by a fourth length, wherein the fourth length may satisfy the following equation:

$$L 4 < L 1$$

where L4 is the fourth length. The first heating element may be configured to operate alone or in combination with the second heating element.

Alternatively or additionally to any of the examples above, in another example, the medical device may further comprise a first sheath removal marking segment. The first sheath removal marking segment may be disposed between the first plurality of marking segments and the second plurality of marking segments.

Alternatively or additionally to any of the examples above, in another example, the first sheath removal marking segment may have a different color or pattern the first plurality of marking segments.

Alternatively or additionally to any of the examples above, in another example, the medical device may further comprise a second sheath removal marking segment disposed distal to the first sheath removal marking segment.

Alternatively or additionally to any of the examples above, in another example, the second sheath removal marking segment may be positioned proximal to and abutting a proximal end of the second heating element.

Alternatively or additionally to any of the examples above, in another example, the second sheath removal marking segment may have a different color or pattern the first plurality of marking segments.

Alternatively or additionally to any of the examples above, in another example, the medical device may further comprise a first final treatment indicator. The first final treatment indicator may provide an indication of a final treatment using the first heating element in combination with the second heating element.

Alternatively or additionally to any of the examples above, in another example, the medical device may further comprise a second final treatment indicator. The second final treatment indicator may provide an indication of a final treatment using the first heating element.

Alternatively or additionally to any of the examples above, in another example, the second final treatment indicator may be distal to the first final treatment indicator.

Alternatively or additionally to any of the examples above, in another example, the first length of the first heating element may be less than the second length of the second heating element.

Alternatively or additionally to any of the examples above, in another example, the third length may satisfy the following equation:

$$L 3 = \frac{( ( L 1 + L 2 ) - OL )}{2}$$

where OL is a treatment overlap length.

Alternatively or additionally to any of the examples above, in another example, the fourth length may satisfy the following equation:

$$L 4 = L 1 - O L$$

where OL is a treatment overlap length.

Alternatively or additionally to any of the examples above, in another example, the third length may satisfy the following equation:

$$L 3 = L 1 - O L$$

where OL is a treatment overlap length.

Alternatively or additionally to any of the examples above, in another example, the first heating element may have a length of about 3 cm (1.181 in).

Alternatively or additionally to any of the examples above, in another example, the second heating element may have a length of about 9 cm (3.543 in).

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1A is cross-sectional view of an illustrative vessel having a healthy venous valve;

FIG. 1B is a cross-section view of an illustrative vessel having an incompetent venous valve;

FIG. 2 is a side view of an illustrative medical device configured to perform a thermal ablation procedure;

FIG. 3 is a schematic side view of the illustrative medical device of FIG. 2 with an introducer sheath;

FIGS. 4-7 are partial cross-sectional views of the medical device of FIG. 2 in a vessel during differing portions of a procedure;

FIG. 8 is a side view of another illustrative medical device configured to perform a thermal ablation procedure;

FIG. 9 is a side view of another illustrative medical device configured to perform a thermal ablation procedure;

FIG. 10 is a side view of another illustrative medical device configured to perform a thermal ablation procedure;

FIG. 11 is a side view of another illustrative medical device configured to perform a thermal ablation procedure; and

FIG. 12 is a side view of another illustrative medical device configured to perform a thermal ablation procedure.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

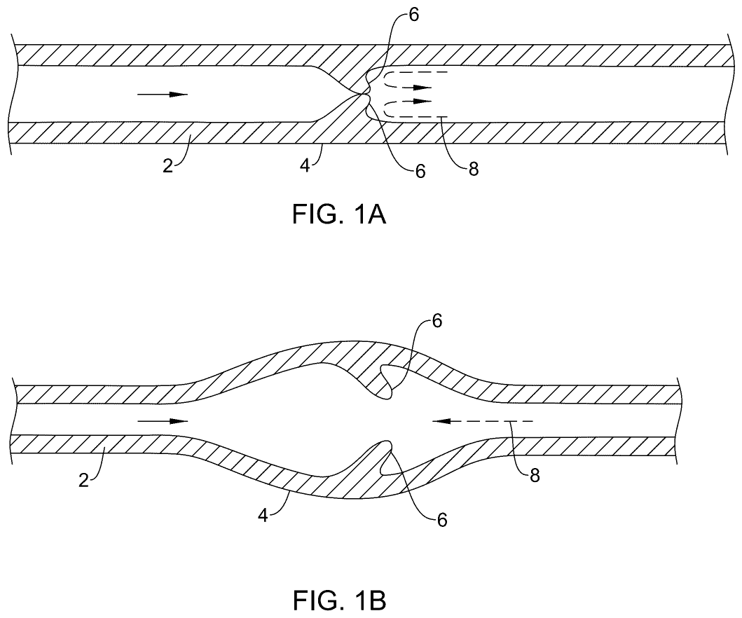

Healthy leg veins contain valves that allow blood to move in one direction from the lower limbs toward the heart. Referring to FIG. 1A, a healthy venous valve 4 is illustrated in a vessel 2. The valve 4 is bicuspid, with opposed cusps 6. In the closed condition, the cusps 6 are drawn together to prevent retrograde flow (arrow 8) of blood. These valves 4 open when blood is flowing toward the heart, and close to prevent venous reflux (also known as venous insufficiency), or the backward flow of blood. When veins weaken and become enlarged, their valves cannot close properly, which leads to venous reflux and impaired drainage of venous blood from the legs. Incompetence of venous valves can contribute to medical conditions such as varicose veins and chronic venous insufficiency, in general. In some cases, pelvic venous insufficiency can result in or contribute to chronic venous insufficiency.

Referring to FIG. 1B, if the valve 4 is incompetent, the cusps 6 do not seal properly and retrograde flow of blood occurs. Venous reflux is most common in the superficial veins. The largest superficial vein is the great saphenous vein, which runs from the top of the foot to the groin, where it terminates at a deep vein. Factors that contribute to venous reflux disease include female gender, heredity, obesity, lack of physical activity, multiple pregnancies, age, past history of blood clots in the legs, and professions that involve long periods of standing. According to population studies, the prevalence of visible tortuous varicose veins, a common indicator of venous reflux disease, is up to 15% for adult men and 25% for adult women. Venous reflux can be classified as either asymptomatic or symptomatic, depending on the degree of severity. Symptomatic venous reflux disease is a more advanced stage of the disease and can have a profound impact on the patient's quality of life. People with symptomatic venous reflux disease may seck treatment due to a combination of symptoms and signs, which may include leg pain and swelling, painful varicose veins; skin changes such as discoloration or inflammation, and/or open skin ulcers. A primary goal of treating symptomatic venous reflux is to eliminate the reflux at its source, such as, for example, the great saphenous vein. If a diseased vein is either closed or removed, blood can reroute into other collateral veins without any known negative consequences to the patient.

There are several treatments available for superficial venous insufficiency and/or pelvic venous disease including, but not necessarily limited to, the use of adhesives delivered endovenously, thermal ablation (radiofrequency, laser, microwave, or steam), mechanochemical ablation (MOCA), foam sclerotherapy, hormone therapy, coils, ligation and stripping, ambulatory phlebectomy, and/or venoactive drugs. In devices that use thermal ablation, it may be desirable to provide a marking scheme on the treatment device to aid in the usability of the device for the clinician. The marking scheme may accommodate varying lengths of treatment elements and/or introducer sheaths. While the present device and/or method is described with respect to treatments for varicose veins, superficial vein insufficiency, and/or pelvic congestion syndrome, it is contemplated that the devices and methods described herein may be used in other anatomical locations and/or therapies requiring heating or ablation of target tissue.

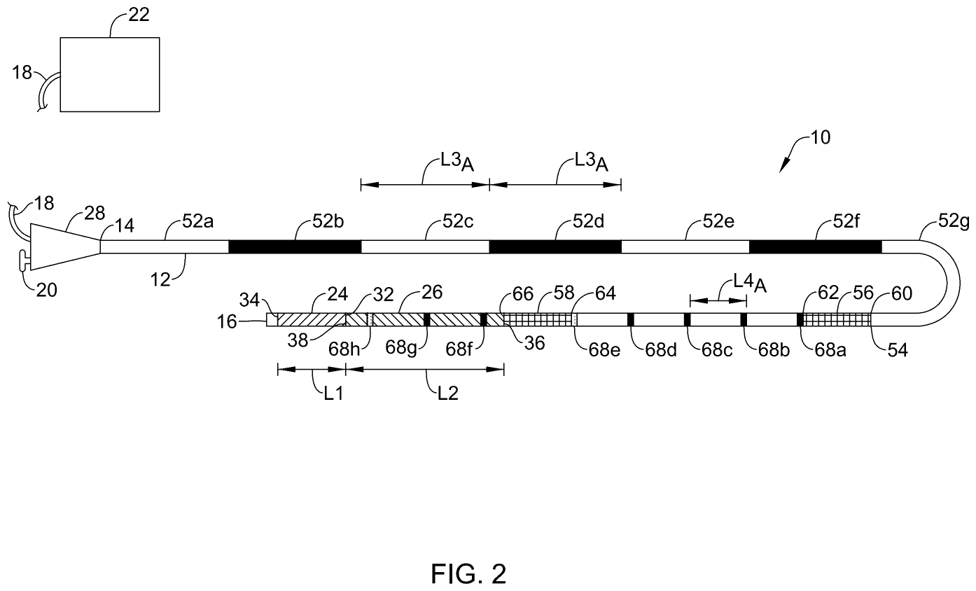

FIG. 2 is a side view of an illustrative medical device 10 configured to perform a thermal ablation procedure. The medical device 10 may be a catheter including a flexible elongate shaft 12. The flexible elongate shaft 12 may extend from a proximal end 14 to a distal end 16. The proximal end 14 may be configured to remain outside of a patient's body. The proximal end 14 of the elongate shaft 12 may include a hub 28 attached thereto for connecting other treatment devices or providing a port 20 for facilitating other treatments. It is contemplated that the stiffness of the elongate shaft 12 may be modified to form a medical device 10 for use in various vessel diameters and various locations within a patient's vasculature. The elongate shaft 12 may further include one or more lumens extending therethrough. For example, the elongate shaft 12 may include a guidewire lumen and/or one or more auxiliary lumens. The lumens may be configured in any way known in the art. For example, the guidewire lumen may extend the entire length of the elongate shaft 12 such as in an over-the-wire catheter or may extend only along a distal portion of the elongate shaft 12 such as in a single operator exchange (SOE) catheter. It is contemplated that if a guidewire is used for access, the guidewire may be proximally retracted prior to treatment unless the guidewire lumen is insulated. These examples are not intended to be limiting, but rather examples of some possible configurations. While not explicitly shown, the medical device 10 may further include temperature sensors/wires, an infusion lumen, radiopaque/echogenic marker bands, fixed guidewire tip, a guidewire lumen, external sheath, and/or other components to facilitate the use and advancement of the medical device 10 within the vasculature.

The medical device 10 may also include an electrical connection or other element 18 for providing power to a heating element disposed around and/or within the flexible elongate shaft 12. A proximal end of the electrical connection 18 may be connected to a control and power element 22, which supplies the necessary electrical energy to activate the one or more heating elements 24, 26 at or near a distal end of the electrical connection 18. While not explicitly shown, the electrical connection 18 may extend through the flexible elongate shaft 12 to the one or more heating elements 24, 26. For example, the electrical connection 18 may be embedded within a side wall of the elongate shaft 12 or may extend through a lumen thereof. These are just some possible configurations. A distal end of the electrical connection 18 may be communicably coupled to the one or more heating elements 24, 26. In some embodiments, the electrical connection 18 may include two or more separate wires or filaments individually coupled to the one or more heating elements 24, 26 such that the one or more heating elements 24, 26 may be individually controlled or activated together. Said differently, the medical device 10 may include more than one electrical connection 18 for providing power from the control and power element 22 to the heating elements 24, 26.

The control and power element 22 may include monitoring elements to monitor parameters such as power, temperature, voltage, impedance pulse size, and/or shape and other suitable parameters as well as suitable controls for performing the desired procedure.

It is contemplated that more than one control and power element 22 may be provided. In some instances, the control and power element 22 may control one or more heating elements 24, 26 such as, but not limited to, one or more radiofrequency (RF) ablation electrodes 24, 26. However, it is contemplated that different types of energy outside the RF spectrum may be used as desired, for example, but not limited to ultrasound, microwave, laser, or the like to perform the ablation or heat treatment. While the term ablation electrode is used herein, it is contemplated that the modulation element and modulation frequency may be selected according to the energy used to perform the ablation. For example, when ultrasound energy is used, an ultrasonic transducer may be selected as the modulation element and modulation frequencies may be in the MHz range.

The medical device 10 may further include one or more heating elements or ablation electrodes 24, 26 disposed on and/or surrounding the outer surface of the elongate shaft 12 adjacent the distal end 16. While the medical device 10 is illustrated as including two heating elements 24, 26, it is contemplated that the medical device 10 may include any number of heating elements 24, 26 desired, such as, but not limited to, one, three, four, or more. If multiple heating elements 24, 26 are provided, the heating elements 24, 26 may be longitudinally and/or circumferentially spaced as desired.

In some examples, a first heating element 24 having a first length L1 extending from a proximal end 32 to a distal end 34 may extend proximally from the distal end 16 of the elongate shaft 12. In some embodiments, the distal end 34 of the first heating element 24 may be proximally spaced from the distal end 16 of the elongate shaft 12. In some cases, the distance from the distal end 16 of the elongate shaft 12 to the distal end 34 of the first heating element 24 may be as short as possible so that the treatment starts as distal to the access site as possible without needing to track further while providing a rounded atraumatic tip, insulation at the forward (or distal) facing end of the first heating element 24, and mechanical robustness to the forward (or distal) facing end of the first heating element 24 to be able to push the medical device 10. However, the distance could be made longer, if so desired. A second heating element 26 having a second length L2 extending from a proximal end 36 to a distal end 38 may be positioned proximal to the first heating element 24. The distal end 38 of the second heating element 26 may abut or contact the proximal end 32 of the first heating element 24 such that when the first and second heating elements 24, 26 are activated together a continuous treatment area results. However, this is not required. In some embodiments, there may be a space or gap between the proximal end 32 of the first heating element 24 and the distal end 38 of the second heating element 26. The second length L2 of the second heating element 26 may be greater than the first length L1 of the first heating element 24. However, this is not required. In some cases, the first length L1 of the first heating element 24 may be greater than the second length L2 of the second heating element 26. In yet other examples, the first length L1 of the first heating element 24 and the second length L2 of the second heating element 26 may be approximately the same. In one illustrative example, the first length L1 of the first heating element 24 may be in the range of about 2 centimeters (cm) (0.787 inches (in)) to about 4 cm (1.575 in), or about 3 cm (1.181 in) and the second length L2 of the second heating element 26 may be in the range of about 8 cm (3.150 in) to about 10 cm (3.937 in), or about 9 cm (3.543 in). This is just an example. Other heating element lengths may be used, as desired.

In some instances, the heating elements 24, 26 may be circumferential electrodes or elements extending around the outer surface of the elongate shaft 12. A circumferential heating element 24, 26 may allow for circumferential treatment or ablation while reducing and/or eliminating the need for circumferential repositioning of the heating elements 24, 26 and/or elongate shaft 12. In some embodiments, the heating elements 24, 26 may not extend around an entirety of the perimeter of the elongate shaft 12. It is contemplated that multiple heating elements 24, 26 may be circumferentially positioned around the perimeter of the elongate shaft 12 to reduce and/or eliminate the need to circumferentially reposition the elongate shaft 12 to perform 360° ablation.

In some embodiments, the heating elements 24, 26 may be formed of one or more separate structures and attached to the elongate shaft 12. For example, each of the heating elements 24, 26 may be machined or stamped from a monolithic piece of material and subsequently bonded or otherwise attached to the elongate shaft 12. In other embodiments, the heating elements 24, 26 may be formed directly on the surface of the elongate shaft 12. For example, the heating elements 24, 26 may be plated, printed, or otherwise deposited on the surface. In some instances, one or more of the heating elements 24, 26 may be sufficiently radiopaque/echogenic so that it also functions as a visual marker under respective imaging modalities. The heating elements 24, 26 may be formed from any suitable material such as, but not limited to, platinum, gold, stainless steel, cobalt alloys, or other non-oxidizing and/or echogenic materials. In some instances, titanium, tantalum, or tungsten may be used. It is contemplated that the heating elements 24, 26 may take any shape desired, such as, but not limited to, square, rectangular, circular, elliptical, cylindrical, semi-cylindrical, tubular, semi-tubular, helically wound coils (open pitch or closed pitch), bidirectionally wound coils, or the like. In some embodiments, the heating elements 24, 26 may have rounded edges in order to reduce the effects of sharp edges on current density. The size and/or shape of the heating elements 24, 26 may be chosen to optimize the current density without increasing the profile of the medical device 10.

As described above, the first and second heating elements 24, 26 may be electrically coupled to the control and power element 22 using separate electrical connections via the electrical connection 18. Each electrical connection may extend proximally from a distal end coupled to an individual heating element 24, 26 to a proximal end coupled to the control and power element 22. In some examples, the electrical connections may extend through the electrical connection 18 or the electrical connection 18 may be formed from the electrical connections. This may allow the first and second heating elements 24, 26 to be activated individually or together. For example, activating only the first heating element 24 may provide a treatment length equal to the first length L1 of the first heating element 24 while activating both the first and the second heating elements 24, 26 may provide a treatment length longer than the first length L1 of the first heating element 24 or equal to the sum of the first length L1 of the first heating element 24 and the second length L2 of the second heating element 26. In other examples, activating only the second heating element 26 may provide yet another treatment length different from the treatment length of the first heating element 24 and the treatment length equal to the sum of the first length L1 of the first heating element 24 and the second length L2 of the second heating element 26.

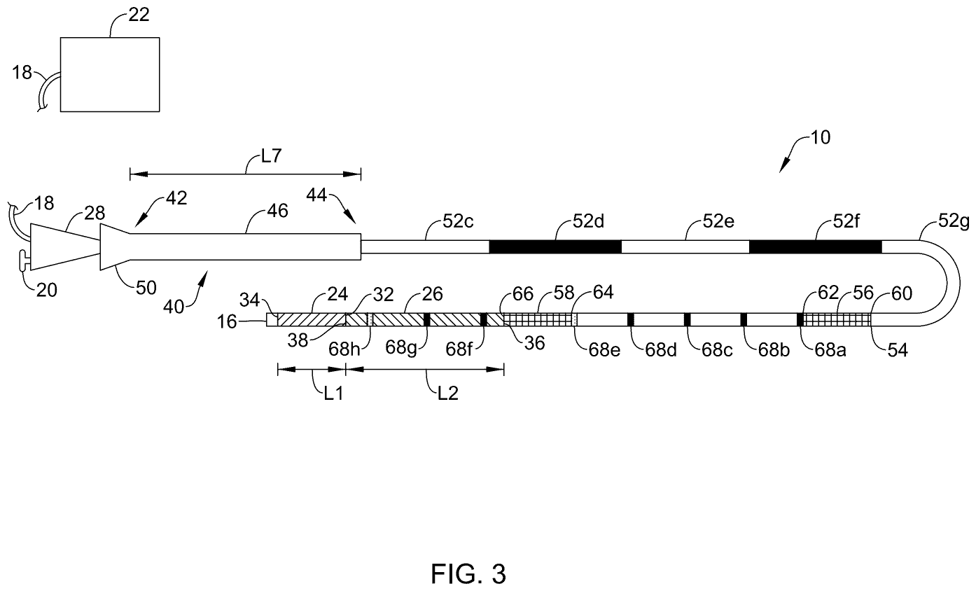

Referring additionally to FIG. 3, which illustrates a schematic side view of the illustrative medical device 10 with an introducer sheath 40, in some embodiments, an introducer sheath 40 may be used for facilitating the passage of the medical device 10 through the introducer sheath 40 and into the vasculature. The introducer sheath 40 includes a proximal end region 42 proximate a proximal end of the introducer sheath 40 and a distal end region 44 proximate a distal end of the introducer sheath 40 that is opposite the proximal end region 42. An elongate shaft 46 of the introducer sheath 40 extends between the proximal end region 42 and the distal end region 44, and the elongate shaft 46 defines a lumen (not explicitly shown) of the introducer sheath 40. The elongate shaft 46 may have a length L7 extending from a proximal end thereof to a distal end thereof. It is contemplated that more than one introducer sheath 40 having differing lengths of elongate shafts 46 may be available to a clinician. The clinician may select an introducer sheath 40 having a length L7 of the elongate shaft 46 that is suitable for the anatomy or the clinician's personal preference. In some illustrative examples, the length L7 of the elongate shaft 46 may be about 7 cm (2.756 in) or about 11 cm (4.331 in). These are just some examples. The elongate shaft 46 may have other lengths L7, as desired.

The introducer sheath 40 includes a proximal opening (not explicitly shown) adjacent the proximal end region 42 and a distal opening (not explicitly shown) adjacent the distal end region 44, with the lumen extending from the proximal opening to the distal opening. The introducer sheath 40, or components thereof, may be formed by various materials, such as polymeric and/or metallic materials. In some instances, the introducer sheath 40, such as the elongate shaft of the introducer sheath 40, may include an additional surface coating, such as but not limited to, silicone, polyethylene terephthalate (PET), or other applicable polymer. While not explicitly shown, in some embodiments, the introducer sheath 40 may include a slit seal, a mechanical iris seal, or the like.

A hemostasis valve hub 50 (hereinafter “hub 50” for brevity) may be provided at the proximal end region 42 to provide access to the lumen of the introducer sheath 40 at approximately the proximal end region 42. The hub 50 may be configured for hemostasis by, for example, helping to prevent blood from leaking out of the introducer sheath 40 during use. For example, the medical device 10 may be inserted through the hub 50 and lumen of the introducer sheath 40 and into the blood vessel, and the hub 50 may maintain hemostasis between the medical device 10, the introducer sheath 40, and the external surroundings. The length of the hub 50 may vary.

For illustrative purposes, a method for treating a patient using two different lengths of heating elements will be described. However, it should be understood that a single length of heating element may be used or more than two differing lengths of heating elements may be used. In an illustrative method, a longer treatment length may be used until the heating element approaches the incision location, at which point the user may switch to a shorter treatment length to continue treatment while protecting the patient. It is contemplated that a mechanical switch or button or an electrical interface may be provided at the control and power element 22 to allow the clinician to switch between treatment lengths. For example, the first and second heating elements 24, 26 may be activated simultaneously to provide a treatment length that is the sum of the length L1 of the first electrode 24 and the length L2 of the second electrode 26. The first and second heating elements 24, 26 may be activated together until the heating elements 24, 26 approach the incision location at which point the first heating element 24 may be activated independently.

To perform a thermal heat treatment of a length of vessel, after preparation of the patient, the introducer sheath 40 may be inserted into the target vessel at an introduction point. Ultrasound or other imaging techniques may be used to guide insertion of the introducer sheath 40. Next, the distal end 16 of the medical device 10 may be inserted through the introducer sheath 40 and advanced through the vasculature to a desired treatment location. The medical device 10 may be advanced with or without a guidewire. The entire leg may be repositioned as necessary to facilitate access. When so provided, a guidewire may be removed from the lumen of the medical device prior to treatment if said lumen is not insulated. In some examples, the distal end 16 of the medical device 10 may be positioned about 2 cm (0.787 in) distal to the crypto-femoral junction (SFJ) without covering the opening of the superficial abdominal wall vein. This is just an example. When the distal end 16 is adjacent to the distalmost end of the target treatment region (relative to the clinician), tumescent anesthesia may be injected into the fascia around the incompetent vein and along the length of an incompetent vein via external, direct injection. The tumescent anesthesia may provide local anesthetic relief as well as pressure along the incompetent vein such that the inner surface of the vein is in closer proximity to the heating elements 24, 26 to optimize heat transfer between the medical device 10 and the vein wall. Next, energy may be supplied to the first and/or second heating element 24, 26. It is contemplated that the clinician may choose to activate the first and second heating elements 24, 26 together to treat a longer length of vessel with each application of energy or the clinician may choose to activate the first heating element 24 individually to treat a smaller length of vessel with each application of energy. As the thermal energy is delivered to the vessel, a cascade of events begin to ultimately occlude the vein. After a predetermined treatment time or a desired temperature is reached, the medical device 10 may be proximally retracted toward the introducer sheath 40 to treat another length of the vessel. In some cases, clinician may choose to reactivate the first and second heating elements 24, 26 again at a same location prior to retraction to treat a region vessel two or more times. The retraction distance may be less than the length L1 of the heating element 24 and/or less than the sum of the lengths L1, L2 of the heating elements 24, 26 used to heat the vessel. This may allow some overlap in the adjacent treatment regions. As will be described in more detail herein, the elongate shaft 12 may include markings configured to help the user withdraw the elongate shaft 12 by a desired amount. The clinician may continue to treat and withdraw the elongate shaft 12 until another marking indicates that the heating element(s) 24, 26 are approaching the introducer sheath 40. Said marking may indicate to the user that the introducer sheath 40 should be removed or withdrawn to avoid damage to the introducer sheath 40, layers of dermis, and to allow the treatment energy to penetrate the vessel. After removal of the introducer sheath 40, the user may continue to activate the first heating element 24 and/or the first and second heating elements 24, 26 and proximally retract or withdraw the elongate shaft 12. This may be repeated until a final treatment marker is visible. At this time, the clinician may perform a final treatment and remove the elongate shaft 12 from the vasculature.

Returning to FIG. 2, the medical device 10 may further include one or more visual indicia disposed along an outer surface of the elongate shaft 12 and/or heating elements 24, 26. The visual indicia may be positioned along the outer surface of the elongate shaft 12 and/or the heating elements 24, 26 at predefined intervals configured to guide the clinician during use of the medical device 10. In some cases, the predefined intervals may vary along a length of the medical device 10. For example, the medical device 10 may include a first marking scheme along a proximal portion thereof and a second marking scheme along a distal portion thereof. Further, different visual indicia may provide the clinician with different information. For example, the visual indicia may provide the user with reference points to facilitate treating lengths of the vessel, moving the medical device 10, and treating another length of the vessel. In another example, the visual indicia may indicate to the user that the introducer sheath 40 should be removed prior to continuing treatment. In yet another example, the visual indicia may indicate to the user that a particular treatment should be a final treatment of a given length. Different patterns, colors, and/or other features may be provided for different types of markings. For example, markings used to guide treatment length may have a first type of marking, markings used to guide removal of the sheath may have a second type of marking, and final treatment markings may have a third type of marking. Each of the first, second, and third types of markings may be different from one another or distinguishable from one another. More than three or fewer than three types of markings may be used, as desired. For example, different marking types may be used to guide removal for two or more different lengths of sheaths.

In one illustrative example, the elongate shaft 12 may include a first plurality of markers or marking segments 52a-g extending distally from the proximal end 14 of the elongate shaft 12 to an intermediate location 54 proximal to the distal end 16 of the elongate shaft 12. The markers 52a-g may alternate in color such that each marker 52a-g is easily distinguishable. For example, the elongate shaft 12 may include a first group of markers 52a, 52c, 52e, 52g having a first color and a second group of markers 52b, 52d, 52f having a second color different from the first color where markers of the first color 52a, 52c, 52c, 52g are separated by markers of the second color 52b, 52d, 52f. It is contemplated that the first plurality of marking segments 52a-g may include other distinguishing features such as, but not limited to, patterns, etchings, alphanumeric indicators, or the like. It is further contemplated that the marking segments 52a-g may have a similar color or distinguishing feature and may be separated by regions free from a marking or by other markers. Thus, each marking segment of the first plurality of marking segments 52a-g may have a length that is less than a desired measuring distance. The markers 52a-g may be separate components extending around and coupled to an outer surface of the elongate shaft 12 or may be formed as a single monolithic structure with the elongate shaft 12, as desired. In one example, the first plurality of marking segments 52a-g may be sections of differently colored tubing joined together. Alternatively, or additionally, the first plurality of marking segments 52a-g may be printed onto an outer surface of the elongate shaft 12.

The first plurality of marking segments 52a-g may each have a third length L3_{A}. In some embodiments, the third length L3_{A} may be defined by Equation 1:

$$L {3}_{A} < \frac{L 1 + L 2}{2} \qquad Equation l$$

where L3_{A} is the third length of each marking segment of the first plurality of marking segments 52a-g, L1 is the first length of the first heating element, and L2 is the second length of the second heating element. Said differently each marking segment of the first plurality of marking segments 52a-g may be less than half the sum of the first length L1 of the first heating element 24 and the second length L2 of the second heating element 26. In some cases, L3_{A} may be determined by optimizing the distance between the proximal and distal ends of a marking segment 52a based on where the physician may start treatment as visualized on the marking segment extending from or positioned adjacent to the hub 50 of the introducer sheath 40. When the clinician is using both the first and second heating elements 24, 26 together, after a treatment is performed, the elongate shaft 12 may be proximally retracted until two adjacent segments have been proximally retracted from the body. This may indicate to the clinician that the heating elements 24, 26 have been displaced from the previously treated region by a desired length.

In some cases, it may be desirable to provide an overlap in adjacent treatment segments. This may ensure that an entirety of the length of the vessel is treated. The desired overlap may be taken into consideration when determining the length of each marking segment of the first plurality of marking segments 52a-g. For example, to obtain a desired overlap in adjacent treatment segments, the third length L3_{A} may be defined by Equation 2:

$$L {3}_{A} = \frac{( ( L 1 + L 2 ) - O L )}{2} \qquad Equation 2$$

where L3_{A} is the third length of a marking segment of the first plurality of marking segments 52a-g, L1 is the first length of the first heating element 24, L2 is the second length of the second heating element 26, and OL is a desired treatment overlap length. In one illustrative example, the first length L1 of the first heating element 24 may be 3 cm (1.181 in), the second length L2 of the second heating element 26 may be 9 cm (3.543 in) and the desired overlap between adjacent treatment segments may be 0.5 cm (0.197 in). In this example, the third length L3_{A} of each marking segment of the first plurality of marking segments 52a-g may be 5.75 cm (2.264 in). This is just one example. Thus, when using both the first and second heating elements 24, 26 together, 12 cm (4.724 in) of vessel may be treated in a single application of energy. The medical device 10 may be retracted by about 11.5 cm (4.528 in) (e.g., the length of two marking segments of the first plurality of marking segments 52a-g) before energy is applied again.

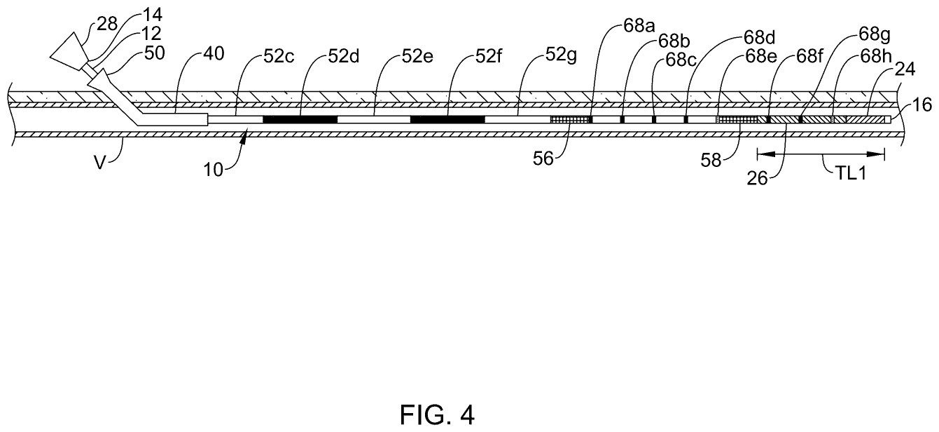

An illustrative method of treatment and the use of various marking segments will be described in more detail with respect to FIGS. 4-7 which illustrate partial cross-sectional views of the medical device 10 in a vessel V during differing portions of a procedure. In FIG. 4, the medical device 10 has been inserted into the vessel V through the introducer sheath 40. In the illustrated embodiment, the medical device 10 has been distally advanced until the proximal end 14 of the elongate shaft 12 is adjacent to or just proximal to the hub 50. However, this is not required. The distance the elongate shaft 12 is inserted may depend on the length of the vessel to be treated. Once the elongate shaft 12 is in position, the first and second heating elements 24, 26 may be activated together to treat a first length TL1 of the vessel V. As the thermal energy is delivered to the vessel V, a cascade of events begin to ultimately occlude the vessel V. For example, the vessel V may shrink and/or collapse on itself to preclude a flow of blood therethrough.

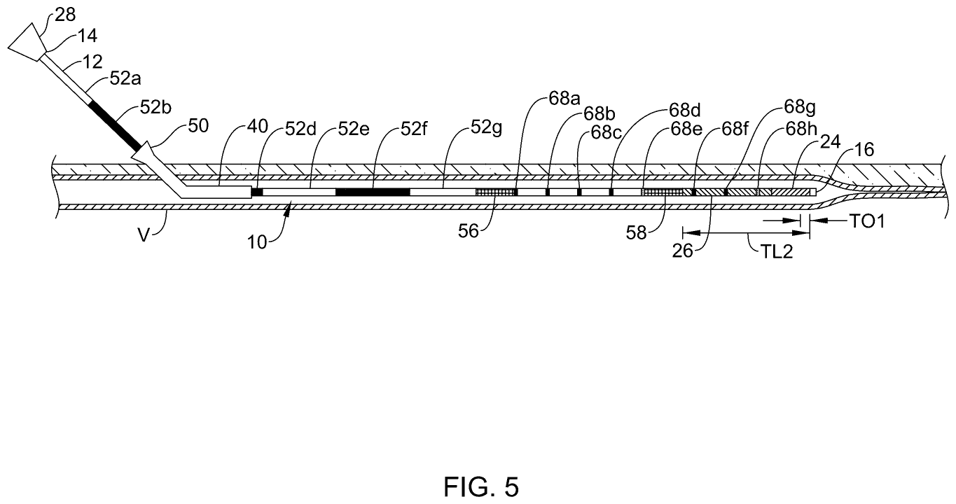

Once the first length TL1 of the vessel has been treated, the medical device 10 is proximally retracted. The clinician may use the first plurality of marking segments 52a-g and the treatment length TL1 to determine how far to retract the medical device 10. For example, as described above, the medical device 10 may be retracted until two marking segments 52a, 52b are removed from the body, as shown in FIG. 5. The first and second heating elements 24, 26 may again be activated together to treat a second length TL2 of the vessel V. The distance of proximal retraction of the medical device 10 may be less than the sum of the lengths L1, L2 of the first and second heating elements 24, 26 such that a first treatment overlap length TO1 occurs adjacent the distal end 16 of the elongate shaft 12 where a length of the vessel V has been treated by both the current treatment and the preceding treatment. Additional treatment overlap lengths may occur for subsequent treatments. The medical device 10 may be repeatedly proximally retracted after each treatment.

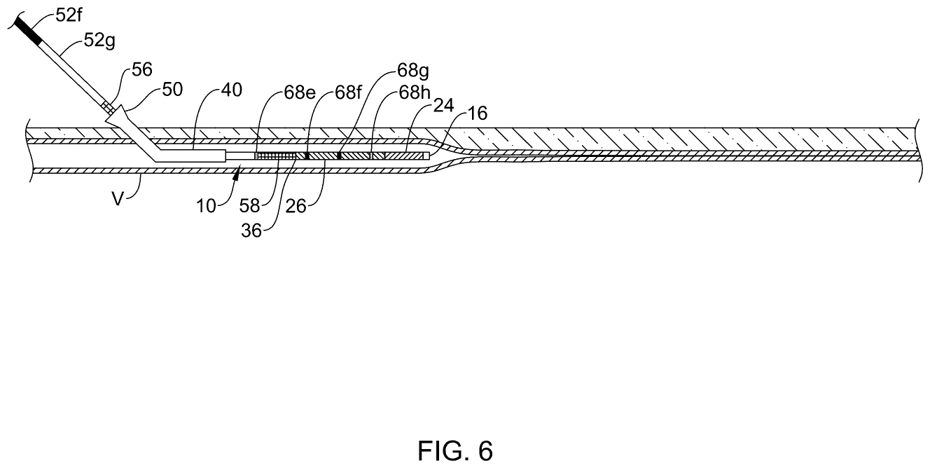

After one or more treatments, the proximal end 36 of the second heating element 26 may approach the introducer sheath 40, as shown in FIG. 6. The elongate shaft 12 may include one or more sheath removal marking segments such as, but not limited to, a proximal sheath removal marking segment 56 and a distal sheath removal marking segment 58 distally spaced from the proximal sheath removal marking segment 56. Generally, the proximal and distal sheath removal marking segments 56, 58 may indicate to the clinician that the introducer sheath 40 should be removed prior to performing any additional treatments to avoid damage to the introducer sheath 40. It is contemplated that as different length of heating elements 24, 26 and/or different lengths of introducer sheaths 40 may be used, the introducer sheath 40 may be removed at different times in the procedure. For example, an 11 cm introducer sheath may extend further into the vessel V than a 7 cm (2.756 in) introducer sheath and thus the proximal end of the heating elements 24, 26 may approach or near the distal end of an 11 cm (4.331 in) introducer sheath with less proximal retraction of the medical device 10 relative to the 7 cm (2.756 in) introducer sheath. Similarly, the proximal end 36 of the second heating element 26 may approach the distal end region 44 of the introducer sheath 40 with less proximal retraction of the medical device 10 than the proximal end 32 of the first heating element 24. In some cases, the sheath removal marking segments 56, 58 may interrupt the first and/or second plurality of marking segments 52a-g, 68a-h. The clinician may use any of the following in that region to help guide proximal retraction of the device 10: approximation by sight or using an O-ring marker on the physical outer diameter of the sheath, allowing overlap of a given region and then go to the next marker (e.g., more overlap in treatment of a segment of the vessel V), or subsequent markings may be spaced to allow counting like a ruler.

Referring additionally to FIGS. 2 and 3, generally, the proximal sheath removal marking segment 56 may be configured to indicate to the clinician that the introducer sheath 40 should be removed when both the first and second heating elements 24, 26 are being activated together. The proximal sheath removal marking segment 56 may extend from a proximal end 60 to a distal end 62 (shown in FIGS. 2 and 3). In some cases, the proximal end 60 of the proximal sheath removal marking segment 56 may be aligned with the intermediate location 54 adjacent to the distal end region of the first plurality of marking segments 52a-g. However, this is not required. The proximal sheath removal marking segment 56 may be sized and shaped to provide a visual indication that prompts the removal of various lengths of introducer sheaths 40. In one illustrative example, the proximal end 60 of the proximal sheath removal marking segment 56 may indicate to the user that the introducer sheath 40 should be removed when an 11 cm (4.331 in) introducer sheath is being used while the distal end 62 of the proximal sheath removal marking segment 56 may indicate to the user that the introducer sheath 40 should be removed when a 7 cm (2.756 in) introducer sheath is being used.

Generally, the distal sheath removal marking segment 58 may be configured to indicate to the clinician that the introducer sheath 40 should be removed when only the first heating element 24 is being activated. When only the first heating element 24 is being activated, the introducer sheath 40 may remain in the body for more treatments than if the first and second heating elements 24, 26 are used together. The distal sheath removal marking segment 58 may be positioned distal to the proximal sheath removal marking segment 56. The distal sheath removal marking segment 58 may extend from a proximal end 64 to a distal end 66 (shown in FIGS. 2 and 3). In some cases, the proximal end 64 of the distal sheath removal marking segment 58 may be longitudinally spaced from the proximal end 60 of the proximal sheath removal marking segment 56 by a length approximately equal to the length L2 of the second heating element 26. However, this is not required. The spacing may be less than or greater than L2, as desired. The distal sheath removal marking segment 58 may be sized and shaped to provide a visual indication that prompts the removal of various lengths of introducer sheaths 40. In one illustrative example, the proximal end 64 of the distal sheath removal marking segment 58 may indicate to the user that the introducer sheath 40 should be removed when an 11 cm (4.331 in) introducer sheath is being used while the distal end 66 of the distal sheath removal marking segment 58 may indicate to the user that the introducer sheath 40 should be removed when a 7 cm (2.756 in) introducer sheath is being used.

The proximal and/or distal sheath removal marking segments 56, 58 may have a different color, pattern, etc. from the first plurality of marking segments 52a-g to indicate to the user that the marking segment is relaying a different type of information. It is contemplated that the proximal and/or distal sheath removal marking segments 56, 58 may include other distinguishing features such as, but not limited to, patterns, etchings, alphanumeric indicators, or the like. The proximal and/or distal sheath removal marking segments 56, 58 may be separate components extending around and coupled to an outer surface of the elongate shaft 12 or may be formed as a single monolithic structure with the elongate shaft 12, as desired. In one example, the proximal and/or distal sheath removal marking segments 56, 58 may be sections of differently colored tubing joined together with other portions of the elongate shaft 12. Alternatively, or additionally, the proximal and/or distal sheath removal marking segments 56, 58 may be printed onto an outer surface of the elongate shaft 12.

The proximal and/or distal sheath removal marking segments 56, 58 may have varying lengths. In one example, the proximal and/or distal sheath removal marking segments 56, 58 may each have a length that is approximately equal to the difference in lengths of the available introducer sheaths 40. For example, if the available introducer sheaths 40 have lengths of 11 cm (4.331 in) and 7 cm (2.756 in), the proximal and/or distal sheath removal marking segments 56, 58 may each have a length of approximately 4 cm (1.575 in). This is just one example. The proximal and/or distal sheath removal marking segments 56, 58 may have lengths longer than 4 cm (1.575 in) or shorter than 4 cm (1.575 in), as desired. Further, the proximal and/or distal sheath removal marking segments 56, 58 need not have the same length.

At or near the intermediate location 54, the elongate shaft 12 may have a change in the marking scheme for the treatment markers. For example, the elongate shaft 12 may include a second plurality of markers or marking segments 68a-68h extending distally from the intermediate location 54. The marking segments 68a-h may each have a similar color or marking scheme to one another, although this is not required. The marking segments 68a-h may be separated by regions free from a marking or by other markers, such as, but not limited to the proximal and/or distal sheath removal marking segments 56, 58. Thus, each marking segment of the second plurality of marking segments 68a-h may have a length that is less than a desired measuring distance. For example, if it is desired to have a marking distance of about 2.5 cm (0.984 in), the length of the marking segments 68a-h may be less than 2.5 cm (0.984 in) to provide separation between each marking segment of the second plurality of marking segments 68a-h. This may help to differentiate the second plurality of marking segments 68a-h from the first plurality of marking segments 52a-g. It is contemplated that the second plurality of marking segments 68a-h may include other distinguishing features such as, but not limited to, colors, patterns, etchings, alphanumeric indicators, or the like. The markers 68a-h may be separate components extending around and coupled to an outer surface of the elongate shaft 12 or may be formed as a single monolithic structure with the elongate shaft 12, as desired. In one example, the second plurality of marking segments 68a-h may be sections of differently colored tubing joined together. Alternatively, or additionally, the second plurality of marking segments 68a-h may be printed onto an outer surface of the elongate shaft 12.

It is contemplated that the second plurality of marking segments 68a-h may be spaced from one another by a fourth length L4_{A} (shown in FIG. 2) that is less than the first length L1 of the first heating element 24. In some cases, the fourth length L4_{A} may be measured from a proximal end of a first marking segment 68b to a proximal end of a second adjacent marking segment 68c, the second marking segment 68c distally spaced from first marking segment 68b. This is just one example. In some cases, the fourth length L4_{A} may be measured from distal end to distal end of adjacent marking segments 68a-h. In other examples, the fourth length L4_{A} may be measured from a distal end of a marking segment to the proximal end of the adjacent marking segment. The fourth length L4_{A} may be defined by Equation 3:

$$L {4}_{A} < L 1 \qquad Equation 3$$

where L4_{A} is the fourth length between each pair of adjacent marking segments of the second plurality of marking segments 68a-h and L1 is the first length of the first heating element 24. When the clinician is using only the first heating element 24, after a treatment is performed, the elongate shaft 12 may be proximally retracted until a next marking segment of the second plurality of marking segments 68a-h is proximally retracted from the body. This may indicate to the clinician that the first heating element 24 has been displaced from the previously treated region by a desired length. In some cases, it may be desirable to provide an overlap in adjacent treatment segments. This may ensure that an entirety of the length of the vessel is treated. The desired overlap may be taken into consideration when determining the length between each pair of adjacent marking segments of the second plurality of marking segments 68a-h. For example, to obtain a desired overlap in adjacent treatment segments when using only the first heating element 24, the fourth length L4_{A} may be defined by Equation 4:

$$L {4}_{A} = L 1 - O L \qquad Equation 4$$

where L4_{A} is the fourth length between each pair of adjacent marking segments of the second plurality of marking segments 68a-h and OL is a desired treatment overlap length. In one illustrative example, the first length L1 of the first heating element 24 may be 3 cm (1.181 in) and the desired overlap between adjacent treatment segments may be 0.5 cm (0.197 in). In this example, the fourth length L4_{A} between each pair of adjacent marking segments of the second plurality of marking segments 68a-h may be 2.5 cm (0.984 in). This is just one example. Thus, when using only the first heating element 24, 3 cm (1.181 in) of vessel may be treated in a single application of energy. The medical device 10 may be retracted by about 2.5 cm (0.984 in) (e.g., the length between adjacent marking segments of the second plurality of marking segments 68a-h) before energy is applied again.

It is contemplated that the second plurality of marking segments 68a-h may also be used to help guide proximal retraction of the medical device 10 when both the first and second heating elements 24, 26 are used together to deliver treatment. In one illustrative example, the first length L1 of the first heating element 24 may be 3 cm (1.181 in), the second length L2 of the second heating element 26 may be 9 cm (3.543 in) such that the treatment length is 12 cm (4.724 in). After the treatment energy has been delivered, the medical device may be retracted by four marking segments of the second plurality of marking segments 68a-h. This may retract the medical device 10 by about 10 cm (3.937 in) (e.g., 2.5 cm multiplied by 4). In this instance, the adjacent treatment regions may overlap by about 2 cm (0.787 in). The number of marking segments of the second plurality of marking segments 68a-h used to guide the proximal retraction when both the first and second heating elements 24, 26 are used may vary depending on the fourth length L4_{A} between adjacent marking segments 68a-h.

In some embodiments, one or more of the second plurality of marking segments 68a-h may include a pattern, color, or other feature to distinguish said marking segment from the other marking segments of the second plurality of marking segments 68a-h. In the illustrated embodiment, two marking segments 68e, 68h have a different pattern from the remaining marking segments 68a-d, 68f-g. These marking segments 68e, 68h having the different pattern may be final treatment indicators, when the introducer sheath 40 has been removed. For example, the final treatment indicators may be positioned such that the clinician is alerted that the heating elements 24, 26 are approaching the tissue and no further application of energy should occur after the current treatment or else unintentional damage to surrounding tissues (e.g., dermis) may occur. It is contemplated that the more proximal final treatment indicator 68e may indicate that if both the first and second heating elements 24, 26 are being used, the current treatment should be the final treatment when the introducer sheath 40 has been removed. The more distal final treatment indicator 68h may indicate that if only the first heating element 24 is being used, the current treatment should be the final treatment when the introducer sheath 40 has been removed. Said differently, once the proximal final treatment indicator 68e is visible a last treatment may be performed using both the first and second heating elements 24, 26 and once the distal final treatment indictor 68h is visible a last treatment may be performed using only the first heating element 24 when the introducer sheath 40 has been removed.

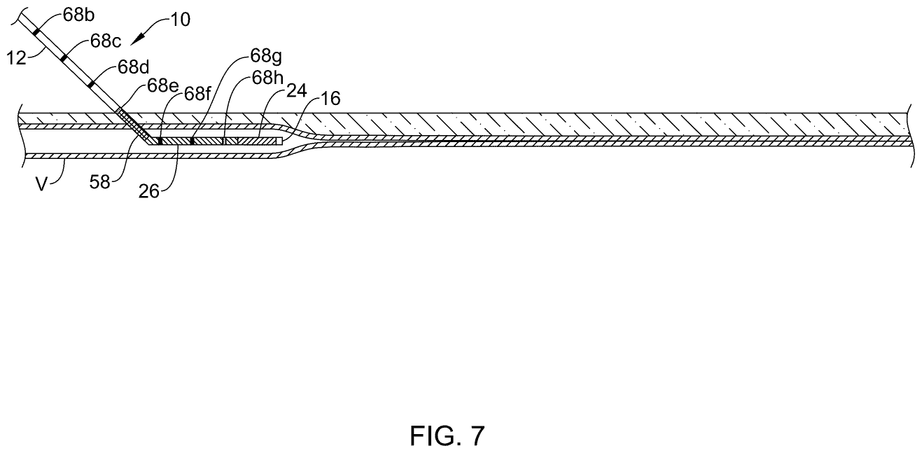

FIG. 7 illustrates a schematic view of the proximal final treatment indicator 68e as exposed from the body. As can be seen in FIG. 7, the introducer sheath 40 has been removed to allow the first and/or second heating elements 24, 26 to treat a portion of the vessel V closest to the introduction location. Once the proximal final treatment indicator 68e is visible, the clinician may perform a treatment using both the first and second heating elements 24, 26 a final time. The clinician may proximally retract the medical device 10 and use only the first heating element 24 to perform additional treatments until the distal final treatment indicator 68h is visible.

It is contemplated that in some cases, the clinician may choose to use both the first and second heating elements 24, 26 for an entirety of the procedure. The first plurality of marking segments 52a-g may guide the proximal retraction of the medical device 10 over a first part of the procedure while the second plurality of marking segments 68a-h guide a second part of the procedure. For example, the clinician may position the distal end 16 of the medical device 10 at the distalmost treatment location (relative to the clinician). The clinician may activate the first and second heating elements 24, 26 to treat a first length of vessel. The first and second heating elements 24, 26 may be deactivated in response to a predetermined condition, such as, but not limited to, obtaining a preset temperature, activating for a predetermined length of time, or the like. The clinician may then proximally retract the medical device 10 using the first plurality of marking segments 52a-g to guide withdrawal length. For example, the clinician may proximally retract the medical device 10 by a length of about two consecutive marking segments 52a-g. The clinician may again activate the first and second heating elements 24, 26 to treat a second length of vessel. A proximal end region of the first treated length of vessel may overlap with a distal end region of the second treated length of vessel such that there is a first length of overlap between adjacent treatment regions. The clinician may repeatedly activate the first and second heating elements 24, 26 and proximally retract the medical device 10 using the first plurality of marking segments 52a-g as a guide until the second plurality of marking segments 68a-h become visible (e.g., the medical device 10 has been proximally retracted by a length sufficient to position a proximal most marking segment 68a of the second plurality of marking segments 68a-h exterior to the body). The clinician may then use the second plurality of marking segments 68a-h to guide the proximal retraction of the medical device. For example, after treatment of a length of the vessel, the clinician may proximally retract the medical device by a length of about four consecutive marking segments 68a-h. The clinician may again activate the first and second heating elements 24, 26 to treat another length of vessel. A proximal end region of the first treated length of vessel may overlap with a distal end region of the second treated length of vessel such that there is a second length of overlap between adjacent treatment regions. It is contemplated that as the second plurality of marking segments 68a-h are spaced differently than the first plurality of marking segments 52a-g, the second length of overlap may be different than the first length of overlap. In the current example, the second length of overlap may be greater than the first length of overlap. However, this is not required. In some examples, the first length of overlap may be greater than the second length of overlap. While performing the incremental treatments, the clinician may remove the introducer sheath 40 when the proximal sheath removal marking segment 56 is visible. The clinician may use the proximal end 60 or the distal end 62 to determine when to remove the introducer sheath 40 based on the length of the introducer sheath 40 used with the procedure. When the proximal final treatment indicator 68e is visible, the clinician may perform a final treatment and remove the medical device 10 from the body.

It is contemplated that in some cases, the clinician may choose to use both the first and second heating elements 24, 26 for a first part or portion of the procedure and only the first heating element 24 for a second part or portion of the procedure. For example, the clinician may use the first and second heating elements 24, 26 together for a first part of the procedure (e.g., further from the introduction point) and switch to using only the first heating element 24 as the heating elements 24, 26 approach the introduction point. The first plurality of marking segments 52a-g may guide the proximal retraction of the medical device 10 over a first part of the procedure while the second plurality of marking segments 68a-h guide a second part of the procedure. For example, the clinician may position the distal end 16 of the medical device 10 at the distalmost treatment location (relative to the clinician). The clinician may activate the first and second heating elements 24, 26 to treat a first length of vessel. The first and second heating elements 24, 26 may be deactivated in response to a predetermined condition, such as, but not limited to, obtaining a preset temperature, activating for a predetermined length of time, or the like. The clinician may then proximally retract the medical device 10 using the first plurality of marking segments 52a-g to guide withdrawal length. For example, the clinician may proximally retract the medical device 10 by a length of about two consecutive marking segments 52a-g. The clinician may again activate the first and second heating elements 24, 26 to treat a second length of vessel. A proximal end region of the first treated length of vessel may overlap with a distal end region of the second treated length of vessel such that there is a first length of overlap between adjacent treatment regions. The clinician may repeatedly activate the first and second heating elements 24, 26 and proximally retract the medical device 10 using the first plurality of marking segments 52a-g as a guide until the second plurality of marking segments 68a-h become visible (e.g., the medical device 10 has been proximally retracted by a length sufficient to position a proximal most marking segment 68a of the second plurality of marking segments 68a-h exterior to the body). The clinician may then switch to using only the first heating element 24 (e.g., by activating a switch or button at the control and power element 22) and then use the second plurality of marking segments 68a-h to guide the proximal retraction of the medical device. For example, after treatment of a length of the vessel, the clinician may proximally retract the medical device by a length of a single consecutive marking segments 68a-h. The clinician may again activate only the first heating element 24 to treat another length of vessel. A proximal end region of the first treated length of vessel may overlap with a distal end region of the second treated length of vessel such that there is a second length of overlap between adjacent treatment regions. It is contemplated that the second length of overlap may be approximately the same as the first length of overlap. However, this is not required. The second length of overlap may be greater than or less than the first length of overlap depending on a spacing of the second plurality of marking segments 68a-h. While performing the incremental treatments, the clinician may remove the introducer sheath 40 when either the proximal sheath removal marking segment 56 or the distal sheath removal marking segment 58 is visible depending on whether the clinician is using both the first and second heating elements 24, 26 or only the first heating element 24 when the proximal and/or distal sheath removal marking segments 56, 58 become visible. The clinician may use the proximal ends 60, 64 or the distal ends 62, 66 to determine when to remove the introducer sheath 40 based on the length of the introducer sheath 40 used with the procedure. When the distal final treatment indicator 68h is visible, the clinician may perform a final treatment and remove the medical device 10 from the body. While the current example describes transitioning from the use of both the first and second heating elements 24, 26 to the use of only the first heating element 24 at the transition between the first plurality of marking segments 52a-g to the second plurality of marking segments 68a-h, this is not required. The clinician may transition between the use of both the first and second heating elements 24, 26 to the use of only the first heating element 24 at any point in the procedure. For example, the clinician may use the first plurality of marking segments 52a-g to approximate a withdrawal distance for use of only the first heating element 24 or the clinician may use one or more of the marking segments of the second plurality of marking segments 68a-h to determine a withdrawal distance for the use of both the first and second heating elements 24, 26.

In yet another example, the clinician may choose to use only the first heating element 24 for an entirety of the procedure. The first plurality of marking segments 52a-g may guide the proximal retraction of the medical device 10 over a first part of the procedure while the second plurality of marking segments 68a-h guide a second part of the procedure. For example, the clinician may position the distal end 16 of the medical device 10 at the distalmost treatment location (relative to the clinician). The clinician may activate the first heating element 24 to treat a first length of vessel. The first heating element 24 may be deactivated in response to a predetermined condition, such as, but not limited to, obtaining a preset temperature, activating for a predetermined length of time, or the like. The clinician may then proximally retract the medical device 10 using the first plurality of marking segments 52a-g to guide withdrawal length. For example, the clinician may proximally retract the medical device 10 by a length of about half of one marking segments 52a-g. The clinician may again activate the first heating element 24 to treat a second length of vessel. A proximal end region of the first treated length of vessel may overlap with a distal end region of the second treated length of vessel such that there is a first length of overlap between adjacent treatment regions. It is contemplated that there may be some variability in the overlap regions due to the estimation of proximal retraction distance. The clinician may repeatedly activate the first heating element 24 and proximally retract the medical device 10 using the first plurality of marking segments 52a-g as a guide until the second plurality of marking segments 68a-h become visible (e.g., the medical device 10 has been proximally retracted by a length sufficient to position a proximal most marking segment 68a of the second plurality of marking segments 68a-h exterior to the body). The clinician may then use the second plurality of marking segments 68a-h to guide the proximal retraction of the medical device. For example, after treatment of a length of the vessel, the clinician may proximally retract the medical device by a length of a single marking segment 68a-h. The clinician may again activate the first heating element 24 to treat another length of vessel. A proximal end region of the first treated length of vessel may overlap with a distal end region of the second treated length of vessel such that there is a second length of overlap between adjacent treatment regions. It is contemplated that as the second plurality of marking segments 68a-h are spaced differently than the first plurality of marking segments 52a-g, the second length of overlap may be different than the first length of overlap. In some examples, the second length of overlap may be greater than the first length of overlap. However, this is not required. In some examples, the first length of overlap may be greater than the second length of overlap. While performing the incremental treatments, the clinician may remove the introducer sheath 40 when the distal sheath removal marking segment 58 is visible. The clinician may use the proximal end 64 or the distal end 66 to determine when to remove the introducer sheath 40 based on the length of the introducer sheath 40 used with the procedure. When the distal final treatment indicator 68h is visible, the clinician may perform a final treatment and remove the medical device 10 from the body.

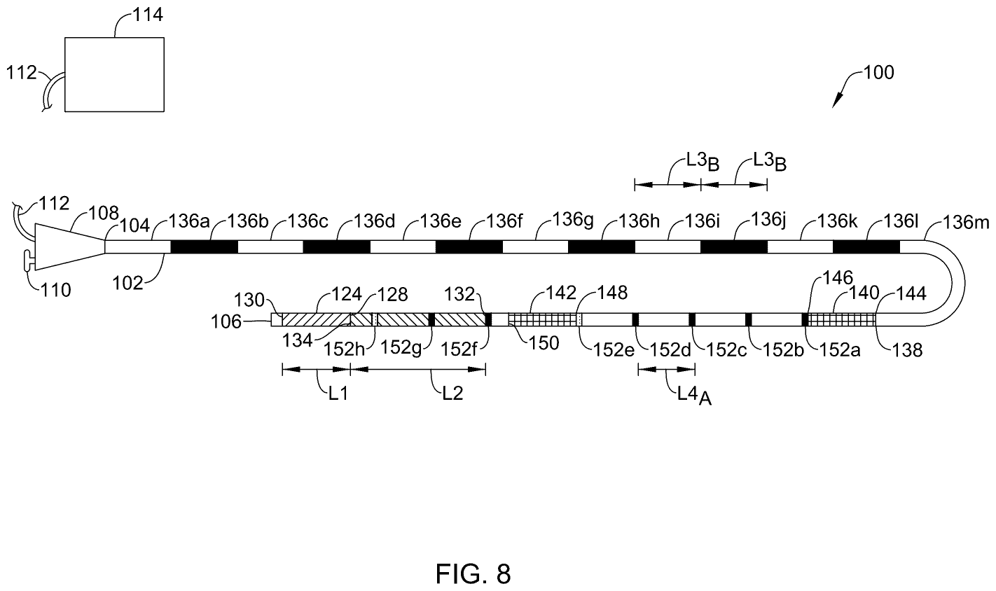

FIG. 8 is a side view of another illustrative medical device 100 configured to perform a thermal ablation procedure. The medical device 100 may be similar in form and function to the medical device 10 described herein with the medical device 100 having an alternative marking scheme. The medical device 100 may be a catheter including a flexible elongate shaft 102. The flexible elongate shaft 102 may extend from a proximal end 104 to a distal end 106. The proximal end 104 of the elongate shaft 102 may include a hub 108 attached thereto for connecting other treatment devices or providing a port 110 for facilitating other treatments. It is contemplated that the stiffness of the elongate shaft 102 may be modified to form a medical device 100 for use in various vessel diameters and various locations within a patient's vasculature. The elongate shaft 102 may further include one or more lumens extending therethrough. For example, the elongate shaft 102 may include a guidewire lumen and/or one or more auxiliary lumens. The lumens may be configured in any way known in the art. For example, the guidewire lumen may extend the entire length of the elongate shaft 102 such as in an over-the-wire catheter or may extend only along a distal portion of the elongate shaft 102 such as in a single operator exchange (SOE) catheter. It is contemplated that if a guidewire is used for access, the guidewire may be proximally retracted prior to treatment unless the guidewire lumen is insulated. These examples are not intended to be limiting, but rather examples of some possible configurations. While not explicitly shown, the medical device 100 may further include temperature sensors/wires, an infusion lumen, radiopaque/echogenic marker bands, fixed guidewire tip, a guidewire lumen, external sheath, and/or other components to facilitate the use and advancement of the medical device 100 within the vasculature.

The medical device 100 may also include an electrical connection or other element 112 similar in form and function to the electrical connection 18 described herein for providing power to a heating element disposed around and/or within the flexible elongate shaft 102. A proximal end of the electrical connection 118 may be connected to a control and power element 114 similar in form and function to the control and power element 22 described herein, which supplies the necessary electrical energy to activate the one or more heating elements 124, 126 at or near a distal end of the electrical connection.

The medical device 100 may further include one or more heating elements or ablation electrodes 124, 126 disposed on and/or surrounding the outer surface of the elongate shaft 102 adjacent the distal end 106. While the medical device 100 is illustrated as including two heating elements 124, 126, it is contemplated that the medical device 100 may include any number of heating elements 124, 126 desired, such as, but not limited to, one, three, four, or more. If multiple heating elements 124, 126 are provided, the heating elements 124, 126 may be longitudinally and/or circumferentially spaced as desired.

In some examples, a first heating element 124 having a first length L1 extending from a proximal end 128 to a distal end 130 may extend proximally from the distal end 106 of the elongate shaft 102. In some embodiments, the distal end 130 of the first heating element 124 may be proximally spaced from the distal end 106 of the elongate shaft 102. In some cases, the distance from the distal end 106 of the elongate shaft 102 to the distal end 130 of the first heating element 124 may be as short as possible so that the treatment starts as distal to the access site as possible without needing to track further while providing a rounded atraumatic tip, insulation at the forward (or distal) facing end of the first heating element 124, and mechanical robustness to the forward (or distal) facing end of the first heating element 124 to be able to push the medical device 100. However, the distance could be made longer, if so desired. A second heating element 126 having a second length L2 extending from a proximal end 132 to a distal end 134 may be positioned proximal to the first heating element 124. The distal end 134 of the second heating element 126 may abut or contact the proximal end 128 of the first heating element 124 such that when the first and second heating elements 124, 126 are activated together a continuous treatment area results. However, this is not required. In some embodiments, there may be a space or gap between the proximal end 128 of the first heating element 124 and the distal end 134 of the second heating element 126. The second length L2 of the second heating element 126 may be greater than the first length L1 of the first heating element 124. However, this is not required. In some cases, the first length L1 of the first heating element 124 may be greater than the second length L2 of the second heating element 126. In yet other examples, the first length L1 of the first heating element 124 and the second length L2 of the second heating element 126 may be approximately the same. In one illustrative example, the first length L1 of the first heating element 124 may be in the range of about 2 cm (0.787 in) to about 4 cm (1.575 in), or about 3 cm (1.181 in) and the second length L2 of the second heating element 126 may be in the range of about 8 cm (3.150 in) to about 10 cm (3.937 in), or about 9 cm (3.543 in). This is just an example. Other heating element lengths may be used, as desired.

In some instances, the heating elements 124, 126 may be circumferential electrodes or elements extending around the outer surface of the elongate shaft 102. A circumferential heating element 124, 126 may allow for circumferential treatment or ablation while reducing and/or eliminating the need for circumferential repositioning of the heating elements 124, 126 and/or elongate shaft 102. In some embodiments, the heating elements 124, 126 may not extend around an entirety of the perimeter of the elongate shaft 102. It is contemplated that multiple heating elements 124, 126 may be circumferentially positioned around the perimeter of the elongate shaft 102 to reduce and/or eliminate the need to circumferentially reposition the elongate shaft 102 to perform 360° ablation.

In some embodiments, the heating elements 124, 126 may be formed of one or more separate structures and attached to the elongate shaft 102. For example, each of the heating elements 124, 126 may be machined or stamped from a monolithic piece of material and subsequently bonded or otherwise attached to the elongate shaft 102. In other embodiments, the heating elements 124, 126 may be formed directly on the surface of the elongate shaft 102. For example, the heating elements 124, 126 may be plated, printed, or otherwise deposited on the surface. In some instances, one or more of the heating elements 124, 126 may be sufficiently radiopaque/echogenic so that it also functions as a visual marker under respective imaging modalities. The heating elements 124, 126 may be formed from any suitable material such as, but not limited to, platinum, gold, stainless steel, cobalt alloys, or other non-oxidizing materials and/or echogenic materials. In some instances, titanium, tantalum, or tungsten may be used. It is contemplated that the heating elements 124, 126 may take any shape desired, such as, but not limited to, square, rectangular, circular, elliptical, cylindrical, semi-cylindrical, tubular, semi-tubular, helically wound coils (open pitch or closed pitch), bidirectionally wound coils, or the like. In some embodiments, the heating elements 124, 126 may have rounded edges in order to reduce the effects of sharp edges on current density. The size and/or shape of the heating elements 124, 126 may be chosen to optimize the current density without increasing the profile of the medical device 100.

As described above, the first and second heating elements 124, 126 may be electrically coupled to the control and power element 114 using separate electrical connections via the electrical connection. Each electrical connection may extend proximally from a distal end coupled to an individual heating element 124, 126 to a proximal end coupled to the control and power element 114. In some examples, the electrical connections may extend through the electrical connection 112 or the electrical connection 112 may be formed from the electrical connections. This may allow the first and second heating elements 124, 126 to be activated individually or together. For example, activating only the first heating element 124 may provide a treatment length equal to the first length L1 of the first heating element 124 while activating both the first and the second heating elements 124, 126 may provide a treatment length longer than the first length L1 of the first heating element 124 or equal to the sum of the first length L1 of the first heating element 124 and the second length L2 of the second heating element 126. In other examples, activating only the second heating element 126 may provide yet another treatment length different from the treatment length of the first heating element 124 and the treatment length equal to the sum of the first length L1 of the first heating element 124 and the second length L2 of the second heating element 126.

While not explicitly shown, the medical device 100 may be used with an introducer sheath, which may be similar in form and function to the introducer sheath 40 described herein. In some illustrative examples, the length of the elongate shaft of the introducer sheath may be about 7 cm (2.756 in) or about 11 cm (4.331 in). These are just some examples. The elongate shaft may have other lengths, as desired.

To perform a thermal heat treatment of a length of vessel, after preparation of the patient, the introducer sheath may be inserted into the target vessel at an introduction point. Next, the distal end 106 of the medical device 100 may be inserted through the introducer sheath and advanced through the vasculature to a desired treatment location. The medical device 100 may be advanced with or without a guidewire. The entire leg may be repositioned as necessary to facilitate access. When so provided, a guidewire may be removed from the lumen of the medical device prior to treatment if said lumen is not insulated. When the distal end 106 is adjacent to the distalmost end of the target treatment region (relative to the clinician), tumescent anesthesia may be injected into the fascia around the incompetent vein and along the length of an incompetent vein via external, direct injection. The tumescent anesthesia may provide local anesthetic relief as well as pressure along the incompetent vein such that the inner surface of the vein is in closer proximity to the heating elements 124, 126 to optimize heat transfer between the medical device 100 and the vein wall. Next, energy may be supplied to the first and/or second heating element 124, 126. It is contemplated that the clinician may choose to activate the first and second heating elements 124, 126 together to treat a longer length of vessel with each application of energy or the clinician may choose to activate the first heating element 124 individually to treat a smaller length of vessel with each application of energy. As the thermal energy is delivered to the vessel, a cascade of events begin to ultimately occlude the vein. After a predetermined treatment time or a desired temperature is reached, the medical device 100 may be proximally retracted towards the introducer sheath to treat another length of the vessel. The retraction distance may be less than the length L1 of the heating element 124 and/or less than the sum of the lengths L1, L2 of the heating elements 124, 126 used to heat the vessel. This may allow some overlap in the adjacent treatment regions. As will be described in more detail herein, the elongate shaft 102 may include markings configured to help the user withdraw the elongate shaft 102 by a desired amount. The clinician may continue to treat and withdraw the elongate shaft 102 until another marking indicates that the heating element(s) 124, 126 are approaching the introducer sheath. Said marking may indicate to the user that the introducer sheath should be removed or withdrawn to avoid damage to the introducer sheath and layers of dermis, and to allow the treatment energy to penetrate the vessel. After removal of the introducer sheath, the user may continue to activate the first heating element 124 and/or the first and second heating elements 124, 126 and proximally retract or withdraw the elongate shaft 102. This may be repeated until a final treatment marker is visible. At this time, the clinician may perform a final treatment and remove the elongate shaft 102 from the vasculature.

The medical device 100 may further include one or more visual indicia disposed along an outer surface of the elongate shaft 102 and/or heating elements 124, 126. The visual indicia may be positioned along the outer surface of the elongate shaft 102 and/or the heating elements 124, 126 at predefined intervals configured to guide the clinician during use of the medical device 100. In some cases, the predefined intervals may vary along a length of the medical device 100. For example, the medical device 100 may include a first marking scheme along a proximal portion thereof and a second marking scheme along a distal portion thereof. Further, different visual indicia may provide the clinician with different information. For example, the visual indicia may provide the user with reference points to facilitate treating lengths of the vessel, moving the medical device 100, and treating another length of the vessel. In another example, the visual indicia may indicate to the user that the introducer sheath should be removed prior to continuing treatment. In yet another example, the visual indicia may indicate to the user that a particular treatment should be a final treatment of a given length. Different patterns, colors, and/or other features may be provided for different types of markings. For example, markings used to guide treatment length may have a first type of marking, markings used to guide removal of the sheath may have a second type of marking, and final treatment markings may have a third type of marking. Each of the first, second, and third types of markings may be different from one another or distinguishable from one another. More than three or fewer than three types of markings may be used, as desired. For example, different marking types may be used to guide removal for two or more different lengths of sheaths.

In one illustrative example, the elongate shaft 102 may include a first plurality of markers or marking segments 136a-m extending distally from the proximal end 104 of the elongate shaft 102 to an intermediate location 138 proximal to the distal end 106 of the elongate shaft 102. The markers 136a-m may alternate in color such that each marker 136a-m is easily distinguishable. For example, the elongate shaft 102 may include a first group of markers 136a, 136c, 136c, 136g, 136i, 136k, 136m having a first color and a second group of markers 136b, 136d, 136f, 136h, 136j, 136l having a second color different from the first color where markers of the first color 136a, 136c, 136c, 136g, 136i, 136k, 136m are separated by markers of the second color 136b, 136d, 136f, 136h, 136j, 136l. It is contemplated that the first plurality of marking segments 136a-m may include other distinguishing features such as, but not limited to, patterns, etchings, alphanumeric indicators, or the like. The markers 136a-m may be separate components extending around and coupled to an outer surface of the elongate shaft 102 or may be formed as a single monolithic structure with the elongate shaft 102, as desired. In one example, the first plurality of marking segments 136a-m may be sections of differently colored tubing joined together. Alternatively, or additionally, the first plurality of marking segments 136a-m may be printed onto an outer surface of the elongate shaft 102.

The first plurality of marking segments 136a-m may each have a third length L3_{B}. As used herein “L3” represents the length of each segment of the first plurality of segments 136a-m while the subscript “A” or “B” may represent segments having differing lengths. In some embodiments, the third length L3_{B} may be defined by Equation 5:

$$L {3}_{B} < \frac{L 1 + L 2}{2} \qquad Equation 5$$

where L3_{B} is the third length of each marking segment of the first plurality of marking segments 136a-m, L1 is the first length of the first heating element, and L2 is the second length of the second heating element. Said differently each marking segment of the first plurality of marking segments 136a-m may be less than half the sum of the first length L1 of the first heating element 124 and the second length L2 of the second heating element 126. In some embodiments, the third length L3_{B} may be more particularly defined relative to the first length L1 of the first heating element 124. For example, in some embodiments, the third length L3_{B} may be defined by Equation 6:

$$L {3}_{B} = L 1 - O L \qquad Equation 6$$

where L3_{B} is the third length of each marking segment of the first plurality of marking segments 136a-m and OL is a desired treatment overlap length when using only the first heating element 124. For example, in the example where the first heating element 124 has a first length L1 of about 3 cm (1.181 in) and there is a desired treatment overlap length OL of about 0.5 cm (0.197 in), each marking segment of the first plurality of marking segments 136a-m may have a length of about 2.5 cm (0.984 in). Thus, when the clinician is using both the first and second heating elements 124, 126 together two or more marking segments of the first plurality of marking segments 136a-m may be used to help guide the proximal retraction of the medical device 10. In one illustrative example, the first length L1 of the first heating element 124 may be about 3 cm (1.181 in), the second length L2 of the second heating element 126 may be about 9 cm (3.543 in) such that when the first and second heating elements 124, 126 are used together, the medical device 100 has a treatment length of about 12 cm (4.724 in). When each marking segment of the first plurality of marking segments 136a-m has a length of about 2.5 cm (0.984 in), the clinician may perform a treatment and then proximally retract the elongate shaft 102 until four adjacent marking segments of the first plurality of marking segments 136a-m have been proximally retracted from the body. This may retract the medical device 100 by about 10 cm (3.937 in) (e.g., 2.5 cm multiplied by 4). In this instance, the adjacent treatment regions may overlap by about 2 cm (0.787 in). The number of marking segments of the second plurality of marking segments 152a-h used to guide the proximal retraction when both the first and second heating elements 124, 126 are used may vary depending on the third length L3_{B} of each marking segment of the first plurality of marking segments 136a-m.

In use, the medical device 100 may be inserted into a vessel through an introducer sheath. The distance the elongate shaft 102 is inserted may depend on the length of the vessel to be treated. Once the elongate shaft 102 is in position, the first and second heating elements 124, 126 may be activated together to treat a first length of the vessel. As the thermal energy is delivered to the vessel, the vessel shrinks and/or collapses on itself to preclude a flow of blood therethrough. Once the first length of the vessel has been treated, the medical device 100 is proximally retracted. The clinician may use the first plurality of marking segments 136a-m and the treatment length to determine how far to retract the medical device 100. For example, the medical device 100 may be retracted until four adjacent marking segments are removed from the body. The first and second heating elements 124, 126 may again be activated together to treat a second length of the vessel. The distance of proximal retraction of the medical device 100 may be less than the sum of the first and second lengths L1, L2 of the first and second heating elements 124, 126 such that a first treatment overlap length occurs adjacent the distal end 106 of the elongate shaft 102 where a length of the vessel has been treated by both the current treatment and the preceding treatment. Additional treatment overlap lengths may occur for subsequent treatments. The medical device 100 may be repeatedly proximally retracted after each treatment.

After one or more treatments, the proximal end 132 of the second heating element 126 may approach the introducer sheath. The elongate shaft 102 may include one or more sheath removal marking segments such as, but not limited to, a proximal sheath removal marking segment 140 and a distal sheath removal marking segment 142 distally spaced from the proximal sheath removal marking segment 140. Generally, the proximal and distal sheath removal marking segments 140, 142 may indicate to the clinician that the introducer sheath should be removed prior to performing any additional treatments to avoid damage to the introducer sheath. It is contemplated that as different length of heating elements 124, 126 and/or different lengths of introducer sheaths may be used, the introducer sheath may be removed at different times in the procedure. For example, an 11 cm (4.331 in) introducer sheath may extend further into the vessel than a 7 cm (2.756 in) introducer sheath and thus the proximal end of the heating elements 124, 126 may approach or near the distal end of an 11 cm (4.331 in) introducer sheath with less proximal retraction of the medical device 100 relative to the 7 cm (2.756 in) introducer sheath. Similarly, the proximal end 132 of the second heating element 126 may approach the distal end region of the introducer sheath with less proximal retraction of the medical device 100 than the proximal end 128 of the first heating element 124.

Generally, the proximal sheath removal marking segment 140 may be configured to indicate to the clinician that the introducer sheath should be removed when both the first and second heating elements 124, 126 are being activated together. The proximal sheath removal marking segment 140 may extend from a proximal end 144 to a distal end 146. In some cases, the proximal end 144 of the proximal sheath removal marking segment 140 may be aligned with the intermediate location 138 adjacent to the distal end region of the first plurality of marking segments 136a-m. However, this is not required. The proximal sheath removal marking segment 140 may be sized and shaped to provide a visual indication that prompts the removal of various lengths of introducer sheaths. In one illustrative example, the proximal end 144 of the proximal sheath removal marking segment 140 may indicate to the user that the introducer sheath should be removed when an 11 cm (4.331 in) introducer sheath is being used while the distal end 146 of the proximal sheath removal marking segment 140 may indicate to the user that the introducer sheath should be removed when a 7 cm (2.756 in) introducer sheath is being used.

Generally, the distal sheath removal marking segment 142 may be configured to indicate to the clinician that the introducer sheath should be removed when only the first heating element 124 is being activated. When only the first heating element 124 is being activated, the introducer sheath may remain in the body for more treatments than if the first and second heating elements 124, 126 are used together. The distal sheath removal marking segment 142 may be positioned distal to the proximal sheath removal marking segment 140. The distal sheath removal marking segment 142 may extend from a proximal end 148 to a distal end 150. In some cases, the proximal end 148 of the distal sheath removal marking segment 142 may be longitudinally spaced from the proximal end 144 of the proximal sheath removal marking segment 140 by a length approximately equal to the length L2 of the second heating element 126. However, this is not required. The spacing may be less than or greater than the second length L2 of the second heating element 126, as desired. The distal sheath removal marking segment 142 may be sized and shaped to provide a visual indication that prompts the removal of various lengths of introducer sheaths. In one illustrative example, the proximal end 148 of the distal sheath removal marking segment 142 may indicate to the user that the introducer sheath should be removed when an 11 cm (4.331 in) introducer sheath is being used while the distal end 150 of the distal sheath removal marking segment 142 may indicate to the user that the introducer sheath should be removed when a 7 cm (2.756 in) introducer sheath is being used.

The proximal and/or distal sheath removal marking segments 140, 142 may have a different color, pattern, etc. from the first plurality of marking segments 136a-m to indicate to the user that the marking segment is relaying a different type of information. It is contemplated that the proximal and/or distal sheath removal marking segments 140, 142 may include other distinguishing features such as, but not limited to, patterns, etchings, alphanumeric indicators, or the like. The proximal and/or distal sheath removal marking segments 140, 142 may be separate components extending around and coupled to an outer surface of the elongate shaft 102 or may be formed as a single monolithic structure with the elongate shaft 102, as desired. In one example, the proximal and/or distal sheath removal marking segments 140, 142 may be sections of differently colored tubing joined together with other portions of the elongate shaft 102. Alternatively, or additionally, the proximal and/or distal sheath removal marking segments 140, 142 may be printed onto an outer surface of the elongate shaft 102.

The proximal and/or distal sheath removal marking segments 140, 142 may have varying lengths. In one example, the proximal and/or distal sheath removal marking segments 140, 142 may each have a length that is approximately equal to the difference in lengths of the available introducer sheaths. For example, if the available introducer sheaths have lengths of 11 cm (4.331 in) and 7 cm (2.756 in), the proximal and/or distal sheath removal marking segments 140, 142 may each have a length of approximately 4 cm (1.575 in). This is just one example. The proximal and/or distal sheath removal marking segments 140, 142 may have lengths longer than 4 cm (1.575 in) or shorter than 4 cm (1.575 in), as desired. Further, the proximal and/or distal sheath removal marking segments 140, 142 need not have the same length.

At or near the intermediate location 138, the elongate shaft 102 may have a change in the marking scheme for the treatment markers. For example, the elongate shaft 102 may include a second plurality of markers or marking segments 152a-152h extending distally from the intermediate location 138. The marking segments 152a-h may each have a similar color or marking scheme to one another, although this is not required. The second plurality of marking segments 152a-h may be similar in form and function to the second plurality of marking segments 68a-h described herein. The marking segments 152a-h may be separated by regions free from a marking or by other markers, such as, but not limited to the proximal and/or distal sheath removal marking segments 140, 142. Thus, each marking segment of the second plurality of marking segments 152a-h may have a length that is less than a desired measuring distance. For example, if it is desired to have a marking distance of about 2.5 cm (0.984 in), the length of the marking segments 152a-h may be less than 2.5 cm (0.984 in) to provide separation between each marking segment of the second plurality of marking segments 152a-h. This may help to differentiate the second plurality of marking segments 152a-h from the first plurality of marking segments 136a-m. It is contemplated that the second plurality of marking segments 152a-h may include other distinguishing features such as, but not limited to, colors, patterns, etchings, alphanumeric indicators, or the like. The markers 152a-h may be separate components extending around and coupled to an outer surface of the elongate shaft 102 or may be formed as a single monolithic structure with the elongate shaft 102, as desired. In one example, the second plurality of marking segments 152a-h may be sections of differently colored tubing joined together with other portions of the elongate shaft 102. Alternatively, or additionally, the second plurality of marking segments 152a-h may be printed onto an outer surface of the elongate shaft 102.

It is contemplated that the second plurality of marking segments 152a-h may be spaced from one another by a fourth length L4_{A} that is less than the first length L1 of the first heating element 124. In some cases, the fourth length L4_{A} may be measured from a proximal end of a first marking segment 152c to a proximal end of a second adjacent marking segment 152d, the second marking segment 152d distally spaced from first marking segment 152c. This is just one example. In some cases, the fourth length L4_{A} may be measured from distal end to distal end of adjacent marking segments 152a-h. In other examples, the fourth length L4_{A} may be measured from a distal end of a marking segment to the proximal end of the adjacent marking segment. The fourth length L4_{A} may be defined by Equation 3. When the clinician is using only the first heating elements 124, after a treatment is performed, the elongate shaft 102 may be proximally retracted until a next marking segment of the second plurality of marking segments 152a-h is proximally retracted from the body. This may indicate to the clinician that the first heating element 124 has been displaced from the previously treated region by a desired length. In some cases, it may be desirable to provide an overlap in adjacent treatment segments. This may ensure that an entirety of the length of the vessel is treated. The desired overlap may be taken into consideration when determining the length between each pair of adjacent marking segments of the second plurality of marking segments 152a-h. For example, to obtain a desired overlap in adjacent treatment segments when using only the first heating element 124, the fourth length L4_{A} may be defined by Equation 4. In one illustrative example, the first length L1 of the first heating element 124 may be 3 cm (1.181 in) and the desired overlap between adjacent treatment segments may be 0.5 cm (0.197 in). In this example, the fourth length L4_{A} between each pair of adjacent marking segments of the second plurality of marking segments 152a-h may be 2.5 cm (0.984 in). This is just one example. Thus, when using only the first heating element 124, 3 cm of vessel may be treated in a single application of energy. The medical device 100 may be retracted by about 2.5 cm (0.984 in) (e.g., the length between adjacent marking segments of the second plurality of marking segments 152a-h) before energy is applied again.

It is contemplated that the second plurality of marking segments 152a-h may also be used to help guide proximal retraction of the medical device 100 when both the first and second heating elements 124, 126 are used together to deliver treatment. In one illustrative example, the first length L1 of the first heating element 124 may be 3 cm (1.181 in), the second length L2 of the second heating element 126 may be 9 cm (3.543 in) such that the treatment length is 12 cm (4.724 in). After the treatment energy has been delivered, the medical device 100 may be retracted by four marking segments of the second plurality of marking segments 152a-h. This may retract the medical device 100 by about 10 cm (3.937 in) (e.g., 2.5 cm multiplied by 4). In this instance, the adjacent treatment regions may overlap by about 2 cm (0.787 in). The number of marking segments of the second plurality of marking segments 152a-h used to guide the proximal retraction when both the first and second heating elements 124, 126 are used may vary depending on the fourth length L4_{A} between adjacent marking segments 152a-h.

In some embodiments, one or more of the second plurality of marking segments 152a-h may include a pattern, color, or other feature to distinguish said marking segment from the other marking segments of the second plurality of marking segments 152a-h. In the illustrated embodiment, two marking segments 152e, 152h have a different pattern from the remaining marking segments 152a-d, 152f-g. These marking segments 152c, 152h having the different pattern may be final treatment indicators when the introducer sheath has been removed. For example, the final treatment indicators may be positioned such that the clinician is alerted that the heating elements 124, 126 are approaching the incision location and no further application of energy should occur after the current treatment or else unintentional damage to surrounding tissues (e.g., dermis) may occur. It is contemplated that the more proximal final treatment indicator 152e may indicate that if both the first and second heating elements 124, 126 are being used, the current treatment should be the final treatment when the introducer sheath has been removed. The more distal final treatment indicated 152h may indicate that if only the first heating element 124 is being used, the current treatment should be the final treatment when the introducer sheath has been removed. Said differently, once the proximal final treatment indicator 152e is visible a last treatment may be performed using both the first and second heating elements 124, 126 when the introducer sheath has been removed and once the distal final treatment indictor 152h is visible a last treatment may be performed using only the first heating element 124 when the introducer sheath has been removed.

It is contemplated that in some cases, the clinician may choose to use both the first and second heating elements 124, 126 for an entirety of the procedure. The first plurality of marking segments 136a-m may guide the proximal retraction of the medical device 100 over a first part of the procedure while the second plurality of marking segments 152a-h guide a second part of the procedure. For example, the clinician may position the distal end 106 of the medical device 100 at the distalmost treatment location (relative to the clinician). The clinician may activate the first and second heating elements 124, 126 to treat a first length of vessel. The first and second heating elements 124, 126 may be deactivated in response to a predetermined condition, such as, but not limited to, obtaining a preset temperature, activating for a predetermined length of time, or the like. The clinician may then proximally retract the medical device 100 using the first plurality of marking segments 136a-m to guide withdrawal length. For example, the clinician may proximally retract the medical device 100 by a length of about four consecutive marking segments 136a-m. The clinician may again activate the first and second heating elements 124, 126 to treat a second length of vessel. A proximal end region of the first treated length of vessel may overlap with a distal end region of the second treated length of vessel such that there is a first length of overlap between adjacent treatment regions. The clinician may repeatedly activate the first and second heating elements 124, 126 and proximally retract the medical device 100 using the first plurality of marking segments 136a-m as a guide until the second plurality of marking segments 152a-h become visible (e.g., the medical device 100 has been proximally retracted by a length sufficient to position a proximal most marking segment 152a of the second plurality of marking segments 152a-h exterior to the body). The clinician may then use the second plurality of marking segments 152a-h to guide the proximal retraction of the medical device. For example, after treatment of a length of the vessel, the clinician may proximally retract the medical device by a length of about four consecutive marking segments 152a-h. The clinician may again activate the first and second heating elements 124, 126 to treat another length of vessel. A proximal end region of the first treated length of vessel may overlap with a distal end region of the second treated length of vessel such that there is a second length of overlap between adjacent treatment regions. It is contemplated that as the second plurality of marking segments 152a-h may be similarly spaced to the first plurality of marking segments 136a-m, the second length of overlap may be approximately the same as the first length of overlap. However, this is not required. In some examples, the first length of overlap may be greater than or less than the second length of overlap. While performing the incremental treatments, the clinician may remove the introducer sheath when the proximal sheath removal marking segment 140 is visible. The clinician may use the proximal end 144 or the distal end 146 to determine when to remove the introducer sheath based on the length of the introducer sheath used with the procedure. When the proximal final treatment indicator 152e is visible, the clinician may perform a final treatment and remove the medical device 100 from the body.

It is contemplated that in some cases, the clinician may choose to use both the first and second heating elements 124, 126 for a first part or portion of the procedure and only the first heating element 124 for a second part or portion of the procedure. For example, the clinician may use the first and second heating elements 124, 126 together for a first part of the procedure (e.g., further from the introduction point) and switch to using only the first heating element 124 as the heating elements 124, 126 approach the introduction point. The first plurality of marking segments 136a-m may guide the proximal retraction of the medical device 100 over a first part of the procedure while the second plurality of marking segments 152a-h guide a second part of the procedure. For example, the clinician may position the distal end 106 of the medical device 100 at the distalmost treatment location (relative to the clinician). The clinician may activate the first and second heating elements 124, 126 to treat a first length of vessel. The first and second heating elements 124, 126 may be deactivated in response to a predetermined condition, such as, but not limited to, obtaining a preset temperature, activating for a predetermined length of time, or the like. The clinician may then proximally retract the medical device 100 using the first plurality of marking segments 136a-m to guide withdrawal length. For example, the clinician may proximally retract the medical device 100 by a length of about four consecutive marking segments 136a-m. The clinician may again activate the first and second heating elements 124, 126 to treat a second length of vessel. A proximal end region of the first treated length of vessel may overlap with a distal end region of the second treated length of vessel such that there is a first length of overlap between adjacent treatment regions. The clinician may repeatedly activate the first and second heating elements 124, 126 and proximally retract the medical device 100 using the first plurality of marking segments 136a-m as a guide until the second plurality of marking segments 152a-h become visible (e.g., the medical device 100 has been proximally retracted by a length sufficient to position a proximal most marking segment 152a of the second plurality of marking segments 152a-h exterior to the body). The clinician may then switch to using only the first heating element 124 (e.g., by activating a switch or button at the control and power element 114) and then use the second plurality of marking segments 152a-h to guide the proximal retraction of the medical device. For example, after treatment of a length of the vessel, the clinician may proximally retract the medical device by a length of a single consecutive marking segments 152a-h. The clinician may again activate only the first heating element 124 to treat another length of vessel. A proximal end region of the first treated length of vessel may overlap with a distal end region of the second treated length of vessel such that there is a second length of overlap between adjacent treatment regions. It is contemplated that the second length of overlap may be less than the first length of overlap. However, this is not required. The second length of overlap may be greater than or approximately equal to the first length of overlap depending on a spacing of the second plurality of marking segments 152a-h. While performing the incremental treatments, the clinician may remove the introducer sheath when either the proximal sheath removal marking segment 140 or the distal sheath removal marking segment 142 is visible depending on whether the clinician is using both the first and second heating elements 124, 126 or only the first heating element 124 when the proximal and/or distal sheath removal marking segments 140, 142 become visible. The clinician may use the proximal ends 144, 148 or the distal ends 146, 150 to determine when to remove the introducer sheath 40 based on the length of the introducer sheath 40 used with the procedure. When the distal final treatment indicator 152h is visible, the clinician may perform a final treatment and remove the medical device 100 from the body. While the current example describes transitioning from the use of both the first and second heating elements 124, 126 to the use of only the first heating element 124 at the transition between the first plurality of marking segments 136a-m to the second plurality of marking segments 152a-h, this is not required. The clinician may transition between the use of both the first and second heating elements 124, 126 to the use of only the first heating element 124 at any point in the procedure. For example, the clinician may use the first plurality of marking segments 136a-m to approximate a withdrawal distance for use of only the first heating element 124 or the clinician may use one or more of the marking segments of the second plurality of marking segments 152a-h to determine a withdrawal distance for the use of both the first and second heating elements 124, 126.

In yet another example, the clinician may choose to use only the first heating element 124 for an entirety of the procedure. The first plurality of marking segments 136a-m may guide the proximal retraction of the medical device 100 over a first part of the procedure while the second plurality of marking segments 152a-h guide a second part of the procedure. For example, the clinician may position the distal end 106 of the medical device 100 at the distalmost treatment location (relative to the clinician). The clinician may activate the first heating element 124 to treat a first length of vessel. The first heating element 124 may be deactivated in response to a predetermined condition, such as, but not limited to, obtaining a preset temperature, activating for a predetermined length of time, or the like. The clinician may then proximally retract the medical device 100 using the first plurality of marking segments 136a-m to guide withdrawal length. For example, the clinician may proximally retract the medical device 100 by one marking segment 136a-m. The clinician may again activate the first heating element 124 to treat a second length of vessel. A proximal end region of the first treated length of vessel may overlap with a distal end region of the second treated length of vessel such that there is a first length of overlap between adjacent treatment regions. The clinician may repeatedly activate the first heating element 124 and proximally retract the medical device 100 using the first plurality of marking segments 136a-m as a guide until the second plurality of marking segments 152a-h become visible (e.g., the medical device 100 has been proximally retracted by a length sufficient to position a proximal most marking segment 152a of the second plurality of marking segments 152a-h exterior to the body). The clinician may then use the second plurality of marking segments 152a-h to guide the proximal retraction of the medical device. For example, after treatment of a length of the vessel, the clinician may proximally retract the medical device by a length of a single marking segment 152a-h. The clinician may again activate the first heating element 124 to treat another length of vessel. A proximal end region of the first treated length of vessel may overlap with a distal end region of the second treated length of vessel such that there is a second length of overlap between adjacent treatment regions. It is contemplated that as the second plurality of marking segments 152a-h are similarly spaced as the first plurality of marking segments 136a-m, the second length of overlap may be approximately the same as the first length of overlap. However, this is not required. In some examples, the second length of overlap may be greater than or less than the first length of overlap. While performing the incremental treatments, the clinician may remove the introducer sheath when the distal sheath removal marking segment 142 is visible. The clinician may use the proximal end 148 or the distal end 150 to determine when to remove the introducer sheath based on the length of the introducer sheath used with the procedure. When the distal final treatment indicator 152h is visible, the clinician may perform a final treatment and remove the medical device 100 from the body.

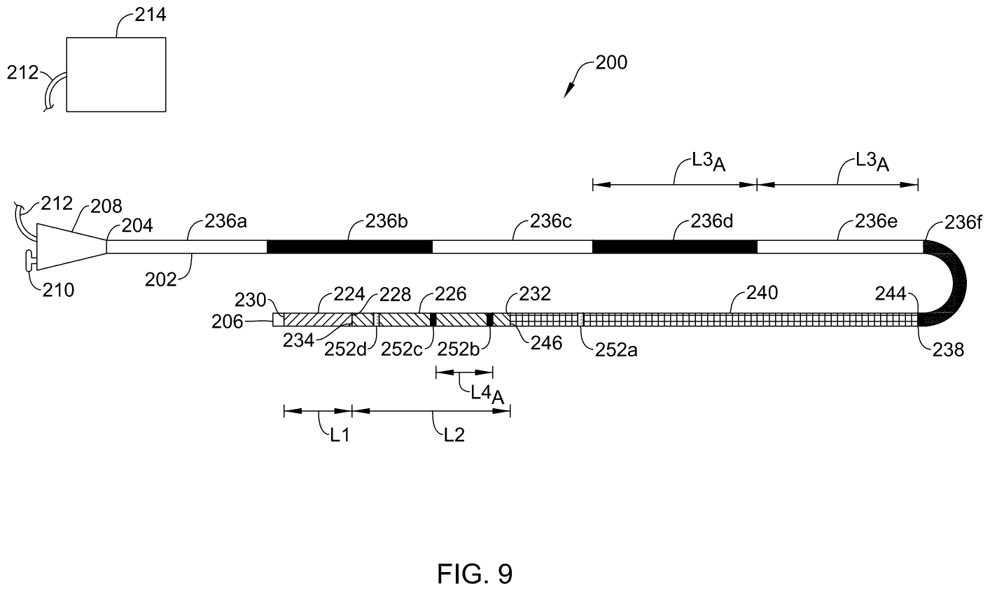

FIG. 9 is a side view of another illustrative medical device 200 configured to perform a thermal ablation procedure. The medical device 200 may be similar in form and function to the medical device 10 described herein with the medical device 200 having an alternative marking scheme. The medical device 200 may be a catheter including a flexible elongate shaft 202. The flexible elongate shaft 202 may extend from a proximal end 204 to a distal end 206. The proximal end 204 of the elongate shaft 202 may include a hub 208 attached thereto for connecting other treatment devices or providing a port 210 for facilitating other treatments. It is contemplated that the stiffness of the elongate shaft 202 may be modified to form a medical device 200 for use in various vessel diameters and various locations within a patient's vasculature. The elongate shaft 202 may further include one or more lumens extending therethrough. For example, the elongate shaft 202 may include a guidewire lumen and/or one or more auxiliary lumens. The lumens may be configured in any way known in the art. For example, the guidewire lumen may extend the entire length of the elongate shaft 202 such as in an over-the-wire catheter or may extend only along a distal portion of the elongate shaft 202 such as in a single operator exchange (SOE) catheter. It is contemplated that if a guidewire is used for access, the guidewire may be proximally retracted prior to treatment unless the guidewire lumen is insulated. These examples are not intended to be limiting, but rather examples of some possible configurations. While not explicitly shown, the medical device 200 may further include temperature sensors/wires, an infusion lumen, radiopaque/echogenic marker bands, fixed guidewire tip, a guidewire lumen, external sheath, and/or other components to facilitate the use and advancement of the medical device 200 within the vasculature.

The medical device 200 may also include an electrical connection or other element 212 similar in form and function to the electrical connection 18 described herein for providing power to a heating element disposed around and/or within the flexible elongate shaft 202. A proximal end of the electrical connection 212 may be connected to a control and power element 214 similar in form and function to the control and power element 22 described herein, which supplies the necessary electrical energy to activate the one or more heating elements 224, 226 at or near a distal end of the electrical connection.

The medical device 200 may further include one or more heating elements or ablation electrodes 224, 226 disposed on and/or surrounding the outer surface of the elongate shaft 202 adjacent the distal end 206. While the medical device 200 is illustrated as including two heating elements 224, 226, it is contemplated that the medical device 200 may include any number of heating elements 224, 226 desired, such as, but not limited to, one, three, four, or more. If multiple heating elements 224, 226 are provided, the heating elements 224, 226 may be longitudinally and/or circumferentially spaced as desired.

In some examples, a first heating element 224 having a first length L1 extending from a proximal end 228 to a distal end 230 may extend proximally from the distal end 206 of the elongate shaft 202. In some embodiments, the distal end 230 of the first heating element 224 may be proximally spaced from the distal end 206 of the elongate shaft 202. In some cases, the distance from the distal end 206 of the elongate shaft 202 to the distal end 230 of the first heating element 224 may be as short as possible so that the treatment starts as distal to the access site as possible without needing to track further while providing a rounded atraumatic tip, insulation at the forward (or distal) facing end of the first heating element 224, and mechanical robustness to the forward (or distal) facing end of the first heating element 224 to be able to push the medical device 200. However, the distance could be made longer, if so desired. A second heating element 226 having a second length L2 extending from a proximal end 232 to a distal end 234 may be positioned proximal to the first heating element 224. The distal end 234 of the second heating element 226 may abut or contact the proximal end 228 of the first heating element 224 such that when the first and second heating elements 224, 226 are activated together a continuous treatment area results. However, this is not required. In some embodiments, there may be a space or gap between the proximal end 228 of the first heating element 224 and the distal end 234 of the second heating element 226. The second length L2 of the second heating element 226 may be greater than the first length L1 of the first heating element 224. However, this is not required. In some cases, the first length L1 of the first heating element 224 may be greater than the second length L2 of the second heating element 226. In yet other examples, the first length L1 of the first heating element 224 and the second length L2 of the second heating element 226 may be approximately the same. In one illustrative example, the first length L1 of the first heating element 224 may be in the range of about 2 cm (0.787 in) to about 4 cm (1.575 in), or about 3 cm (1.181 in) and the second length L2 of the second heating element 226 may be in the range of about 8 cm (3.150 in) to about 10 cm (3.937 in), or about 9 cm (3.543 in). This is just an example. Other heating element lengths may be used, as desired.

In some instances, the heating elements 224, 226 may be circumferential electrodes or elements extending around the outer surface of the elongate shaft 202. A circumferential heating element 224, 226 may allow for circumferential treatment or ablation while reducing and/or eliminating the need for circumferential repositioning of the heating elements 224, 226 and/or elongate shaft 202. In some embodiments, the heating elements 224, 226 may not extend around an entirety of the perimeter of the elongate shaft 202. It is contemplated that multiple heating elements 224, 226 may be circumferentially positioned around the perimeter of the elongate shaft 202 to reduce and/or eliminate the need to circumferentially reposition the elongate shaft 202 to perform 360° ablation.

In some embodiments, the heating elements 224, 226 may be formed of one or more separate structures and attached to the elongate shaft 202. For example, each of the heating elements 224, 226 may be machined or stamped from a monolithic piece of material and subsequently bonded or otherwise attached to the elongate shaft 202. In other embodiments, the heating elements 224, 226 may be formed directly on the surface of the elongate shaft 202. For example, the heating elements 224, 226 may be plated, printed, or otherwise deposited on the surface. In some instances, one or more of the heating elements 224, 226 may be sufficiently radiopaque/echogenic so that it also functions as a visual marker under respective imaging modalities. The heating elements 224, 226 may be formed from any suitable material such as, but not limited to, platinum, gold, stainless steel, cobalt alloys, or other non-oxidizing and/or echogenic materials. In some instances, titanium, tantalum, or tungsten may be used. It is contemplated that the heating elements 224, 226 may take any shape desired, such as, but not limited to, square, rectangular, circular, elliptical, cylindrical, semi-cylindrical, tubular, semi-tubular, helically wound coils (open pitch or closed pitch), bidirectionally wound coils, or the like. In some embodiments, the heating elements 224, 226 may have rounded edges in order to reduce the effects of sharp edges on current density. The size and/or shape of the heating elements 224, 226 may be chosen to optimize the current density without increasing the profile of the medical device 200.

As described above, the first and second heating elements 224, 226 may be electrically coupled to the control and power element 214 using separate electrical connections via the electrical connection 212. Each electrical connection may extend proximally from a distal end coupled to an individual heating element 224, 226 to a proximal end coupled to the control and power element 214. In some examples, the electrical connections may extend through the electrical connection 214 or the electrical connection 214 may be formed from the electrical connections. This may allow the first and second heating elements 224, 226 to be activated individually or together. For example, activating only the first heating element 224 may provide a treatment length equal to the first length L1 of the first heating element 224 while activating both the first and the second heating elements 224, 226 may provide a treatment length longer than the first length L1 of the first heating element 224 or equal to the sum of the first length L1 of the first heating element 224 and the second length L2 of the second heating element 226. In other examples, activating only the second heating element 226 may provide yet another treatment length different from the treatment length of the first heating element 224 and the treatment length equal to the sum of the first length L1 of the first heating element 224 and the second length L2 of the second heating element 226.

While not explicitly shown, the medical device 200 may be used with an introducer sheath, which may be similar in form and function to the introducer sheath 40 described herein. In some illustrative examples, the length of the elongate shaft of the introducer sheath may be about 7 cm (2.756 in) or about 11 cm (4.331 in). These are just some examples. The elongate shaft of the introducer sheath may have other lengths, as desired.

Next, the distal end 206 of the medical device 200 may be inserted through the introducer sheath and advanced through the vasculature to a desired treatment location. The medical device 200 may be advanced with or without a guidewire. The entire leg may be repositioned as necessary to facilitate access. When so provided, a guidewire may be removed from the lumen of the medical device prior to treatment if said lumen is not insulated. When the distal end 206 is adjacent to the distalmost end of the target treatment region (relative to the clinician), tumescent anesthesia may be injected into the fascia around the incompetent vein and along the length of an incompetent vein via external, direct injection. The tumescent anesthesia may provide local anesthetic relief as well as pressure along the incompetent vein such that the inner surface of the vein is in closer proximity to the heating elements 224, 226 to optimize heat transfer between the medical device 200 and the vein wall. Next, energy may be supplied to the first and/or second heating element 224, 226. It is contemplated that the clinician may choose to activate the first and second heating elements 224, 226 together to treat a longer length of vessel with each application of energy or the clinician may choose to activate the first heating element 224 individually to treat a smaller length of vessel with each application of energy. As the thermal energy is delivered to the vessel, a cascade of events begin to ultimately occlude the vein. After a predetermined treatment time or a desired temperature is reached, the medical device 200 may be proximally retracted toward the introducer sheath to treat another length of the vessel. The retraction distance may be less than the length L1 of the heating element 224 and/or less than the sum of the lengths L1, L2 of the heating elements 224, 226 used to heat the vessel. This may allow some overlap in the adjacent treatment regions. As will be described in more detail herein, the elongate shaft 202 may include markings configured to help the user withdraw the elongate shaft 202 by a desired amount. The clinician may continue to treat and withdraw the elongate shaft 202 until another marking indicates that the heating element(s) 224, 226 are approaching the introducer sheath. Said marking may indicate to the user that the introducer sheath should be removed or withdrawn to avoid damage to the introducer sheath and layers of dermis, and to allow the treatment energy to penetrate the vessel. After removal of the introducer sheath, the user may continue to activate the first heating element 224 and/or the first and second heating elements 224, 226 and proximally retract or withdraw the elongate shaft 202. This may be repeated until a final treatment marker is visible. At this time, the clinician may perform a final treatment and remove the elongate shaft 202 from the vasculature.

The medical device 200 may further include one or more visual indicia disposed along an outer surface of the elongate shaft 202 and/or heating elements 224, 226. The visual indicia may be positioned along the outer surface of the elongate shaft 202 and/or the heating elements 224, 226 at predefined intervals configured to guide the clinician during use of the medical device 200. In some cases, the predefined intervals may vary along a length of the medical device 200. For example, the medical device 200 may include a first marking scheme along a proximal portion thereof and a second marking scheme along a distal portion thereof. Further, different visual indicia may provide the clinician with different information. For example, the visual indicia may provide the user with reference points to facilitate treating lengths of the vessel, moving the medical device 200, and treating another length of the vessel. In another example, the visual indicia may indicate to the user that the introducer sheath should be removed prior to continuing treatment. In the illustrated embodiment, the elongate shaft 202 may include markings for the use of a single length of introducer sheath. In yet another example, the visual indicia may indicate to the user that a particular treatment should be a final treatment of a given length. Different patterns, colors, and/or other features may be provided for different types of markings. For example, markings used to guide treatment length may have a first type of marking, markings used to guide removal of the sheath may have a second type of marking, and final treatment markings may have a third type of marking. Each of the first, second, and third types of markings may be different from one another or distinguishable from one another. More than three or fewer than three types of markings may be used, as desired. For example, different marking types may be used to guide removal for two or more different lengths of sheaths.

In one illustrative example, the elongate shaft 202 may include a first plurality of markers or marking segments 236a-f extending distally from the proximal end 204 of the elongate shaft 202 to an intermediate location 238 proximal to the distal end 206 of the elongate shaft 202. The markers 236a-f may alternate in color such that each marker 236a-f is easily distinguishable. For example, the elongate shaft 202 may include a first group of markers 236a, 236c, 236c having a first color and a second group of markers 236b, 236d, 236f having a second color different from the first color where markers of the first color 236a, 236c, 236e are separated by markers of the second color 236b, 236d, 236f. It is contemplated that the first plurality of marking segments 236a-f may include other distinguishing features such as, but not limited to, patterns, etchings, alphanumeric indicators, or the like. The markers 236a-f may be separate components extending around and coupled to an outer surface of the elongate shaft 202 or may be formed as a single monolithic structure with the elongate shaft 202, as desired. In one example, the first plurality of marking segments 236a-f may be sections of differently colored tubing joined together. Alternatively, or additionally, the first plurality of marking segments 236a-f may be printed onto an outer surface of the elongate shaft 202. The first plurality of marking segments 236a-g may be similar in size and shape to the first plurality of marking segments 52a-g described herein. For example, the first plurality of marking segments 236a-f may each have a third length L3_{A} defined by Equations 1 or 2.

In use, the medical device 200 may be inserted into a vessel through an introducer sheath. The distance the elongate shaft 202 is inserted may depend on the length of the vessel to be treated. Once the elongate shaft 202 is in position, the first and second heating elements 224, 226 may be activated together to treat a first length of the vessel. As the thermal energy is delivered to the vessel, the vessel shrinks and/or collapses on itself to preclude a flow of blood therethrough. Once the first length of the vessel has been treated, the medical device 200 is proximally retracted. The clinician may use the first plurality of marking segments 236a-f to determine how far to retract the medical device 200. For example, the medical device 200 may be retracted until two adjacent marking segments are removed from the body. The first and second heating elements 224, 226 may again be activated together to treat a second length of the vessel. The distance of proximal retraction of the medical device 200 may be less than the sum of the first and second lengths L1, L2 of the first and second heating elements 224, 226 such that a first treatment overlap length occurs adjacent the distal end 206 of the elongate shaft 202 where a length of the vessel has been treated by both the current treatment and the preceding treatment. Additional treatment overlap lengths may occur for subsequent treatments. The medical device 200 may be repeatedly proximally retracted after each treatment.

After one or more treatments, the proximal end 232 of the second heating element 226 may approach the introducer sheath. The elongate shaft 202 may include one or more sheath removal marking segments 240. In the illustrated embodiment, the elongate shaft 202 may include only a single sheath removal marking segment 240 configured to guide a clinician for removal of an introducer sheath of a single length. For illustrative purposes, the introducer sheath may have an elongate shaft having a length of about 7 cm (2.756 in). However, this is just an example, the elongate shaft 202 may include markings for introducer sheaths of other lengths. However, the sheath removal marking segment 240 may be proximally or distally displaced on the elongate shaft 202 depending on whether the introducer sheath is longer than 7 cm (2.756 in) or shorter than 7 cm (2.756 in). Generally, the sheath removal marking segment 240 may indicate to the clinician that the introducer sheath should be removed prior to performing any additional treatments to avoid damage to the introducer sheath. It is contemplated that as different length of heating elements 224, 226 and/or different lengths of introducer sheaths may be used, the introducer sheath may be removed at different times in the procedure. For example, an 11 cm (4.331 in) introducer sheath may extend further into the vessel than a 7 cm (2.756 in) introducer sheath and thus the proximal end of the heating elements 224, 226 may approach or near the distal end of an 11 cm (4.331 in) introducer sheath with less proximal retraction of the medical device 200 relative to the 7 cm (2.756 in) introducer sheath. Similarly, the proximal end 232 of the second heating element 226 may approach the distal end region of the introducer sheath with less proximal retraction of the medical device 200 than the proximal end 228 of the first heating element 224.

The sheath removal marking segment 240 may extend from a proximal end 244 to a distal end 246. In some cases, the proximal end 244 of the sheath removal marking segment 240 may be aligned with the intermediate location 238 adjacent to the distal end region of the first plurality of marking segments 236a-f. However, this is not required. The sheath removal marking segment 240 may be sized and shaped to provide a visual indication that prompts the removal of various lengths of introducer sheaths. In one illustrative example, the proximal end 244 of the sheath removal marking segment 240 may indicate to the user that the introducer sheath should be removed when both the first and second heating elements 224, 226 are being used while the distal end 246 of the sheath removal marking segment 240 may indicate to the user that the introducer sheath should be removed when only the first heating element 224 is being used.

The sheath removal marking segment 240 may have a different color, pattern, etc. from the first plurality of marking segments 236a-f to indicate to the user that the marking segment is relaying a different type of information. It is contemplated that the sheath removal marking segment 240 may include other distinguishing features such as, but not limited to, patterns, etchings, alphanumeric indicators, or the like. The sheath removal marking segment 240 may be a separate component extending around and coupled to an outer surface of the elongate shaft 202 or may be formed as a single monolithic structure with the elongate shaft 202, as desired. In one example, the sheath removal marking segment 240 may be a section of differently colored tubing joined together with other portions of the elongate shaft 202. Alternatively, or additionally, the sheath removal marking segment 240 may be printed onto an outer surface of the elongate shaft 202. The sheath removal marking segment 240 may have varying lengths. In one example, the sheath removal marking segment 240 may have a length that is approximately equal to the second length L2 of the second heating element 226. This is just one example. The sheath removal marking segment 240 may have other lengths, as desired.

At or near the intermediate location 238, the elongate shaft 202 may have a change in the marking scheme for the treatment markers. For example, the elongate shaft 202 may include a second plurality of markers or marking segments 252a-d extending distally from the intermediate location 238. The marking segments 252a-d may each have a similar color or marking scheme to one another, although this is not required. The marking segments 252a-d may be separated by regions free from a marking or by other markers, such as, but not limited to the sheath removal marking segment 240. Thus, each marking segment of the second plurality of marking segments 252a-d may have a length that is less than a desired measuring distance. For example, if it is desired to have a marking distance of about 2.5 cm (0.984 in), the length of the marking segments 252a-d may be less than 2.5 cm (0.984 in) to provide separation between each marking segment of the second plurality of marking segments 252a-d. This may help to differentiate the second plurality of marking segments 252a-d from the first plurality of marking segments 236a-f. It is contemplated that the second plurality of marking segments 252a-d may include other distinguishing features such as, but not limited to, colors, patterns, etchings, alphanumeric indicators, or the like. The markers 252a-d may be separate components extending around and coupled to an outer surface of the elongate shaft 202 or may be formed as a single monolithic structure with the elongate shaft 202, as desired. In one example, the second plurality of marking segments 252a-d may be sections of differently colored tubing joined together with other portions of the elongate shaft 202. Alternatively, or additionally, the second plurality of marking segments 252a-d may be printed onto an outer surface of the elongate shaft 202.

It is contemplated that the second plurality of marking segments 252a-d may be spaced from one another by a fourth length L4 that is less than the first length L1 of the first heating element 224. The second plurality of marking segments 252a-d may be similar in form and function to the second plurality of marking segments 68a-g described herein. For example, the fourth length L4 of the space separating the second plurality of marking segments 252a-d may be defined by Equations 3 or 4.

It is contemplated that the second plurality of marking segments 252a-d may also be used to help guide proximal retraction of the medical device 200 when both the first and second heating elements 224, 226 are used together to deliver treatment. In one illustrative example, the first length L1 of the first heating element 224 may be 3 cm (1.181 in), the second length L2 of the second heating element 226 may be 9 cm (3.543 in) such that the treatment length is 12 cm (4.724 in). After the treatment energy has been delivered, the medical device 200 may be retracted by four marking segments of the second plurality of marking segments 252a-d. This may retract the medical device 200 by about 10 cm (3.937 in) (e.g., 2.5 cm multiplied by 4). In this instance, the adjacent treatment regions may overlap by about 2 cm (0.787 in). The number of marking segments of the second plurality of marking segments 252a-d used to guide the proximal retraction when both the first and second heating elements 224, 226 are used may vary depending on the fourth length L4 between adjacent marking segments 252a-d.

In some embodiments, one or more of the second plurality of marking segments 252a-d may include a pattern, color, or other feature to distinguish said marking segment from the other marking segments of the second plurality of marking segments 252a-d. In the illustrated embodiment, two marking segments 252a, 252d have a different pattern from the remaining marking segments 252b, 252c. These marking segments 252a, 252d having the different pattern may be final treatment indicators when the introducer sheath has been removed. For example, the final treatment indicators may be positioned such that the clinician is alerted that the heating elements 224, 226 are approaching the incision location and no further application of energy should occur after the current treatment or else unintentional damage to surrounding tissues (e.g., dermis) may occur. It is contemplated that the more proximal final treatment indicator 252a may indicate that if both the first and second heating elements 224, 226 are being used, the current treatment should be the final treatment. The more distal final treatment indicated 252d may indicate that if only the first heating element 224 is being used, the current treatment should be the final treatment. Said differently, once the proximal final treatment indicator 252a is visible a last treatment may be performed using both the first and second heating elements 224, 226 and once the distal final treatment indictor 252d is visible a last treatment may be performed using only the first heating element 224.

It is contemplated that in some cases, the clinician may choose to use both the first and second heating elements 224, 226 for an entirety of the procedure. The first plurality of marking segments 236a-f may guide the proximal retraction of the medical device 200 over a first part of the procedure while the second plurality of marking segments 252a-d guide a second part of the procedure. For example, the clinician may position the distal end 206 of the medical device 200 at the distalmost treatment location (relative to the clinician). The clinician may activate the first and second heating elements 224, 226 to treat a first length of vessel. The first and second heating elements 224, 226 may be deactivated in response to a predetermined condition, such as, but not limited to, obtaining a preset temperature, activating for a predetermined length of time, or the like. The clinician may then proximally retract the medical device 200 using the first plurality of marking segments 236a-f to guide withdrawal length. For example, the clinician may proximally retract the medical device 200 by a length of about two consecutive marking segments 236a-f. The clinician may again activate the first and second heating elements 224, 226 to treat a second length of vessel. A proximal end region of the first treated length of vessel may overlap with a distal end region of the second treated length of vessel such that there is a first length of overlap between adjacent treatment regions. The clinician may repeatedly activate the first and second heating elements 224, 226 and proximally retract the medical device 200 using the first plurality of marking segments 236a-f as a guide until the second plurality of marking segments 252a-d become visible (e.g., the medical device 200 has been proximally retracted by a length sufficient to position a proximal most marking segment 252a of the second plurality of marking segments 252a-d exterior to the body). The clinician may then use the second plurality of marking segments 252a-d to guide the proximal retraction of the medical device. For example, after treatment of a length of the vessel, the clinician may proximally retract the medical device by a length of about four consecutive marking segments 252a-d. The clinician may again activate the first and second heating elements 224, 226 to treat another length of vessel. A proximal end region of the first treated length of vessel may overlap with a distal end region of the second treated length of vessel such that there is a second length of overlap between adjacent treatment regions. It is contemplated that as the second plurality of marking segments 252a-d arc spaced differently from the first plurality of marking segments 236a-f, the second length of overlap may be different from the first length of overlap. However, this is not required. In some examples, the first length of overlap may be the same as the second length of overlap. While performing the incremental treatments, the clinician may remove the introducer sheath when the sheath removal marking segment 240 is visible. The clinician may use the proximal end 244 to determine when to remove the introducer sheath when both the first and second heating elements 224, 226 are being used. When the proximal final treatment indicator 252a is visible, the clinician may perform a final treatment and remove the medical device 200 from the body.

It is contemplated that in some cases, the clinician may choose to use both the first and second heating elements 224, 226 for a first part or portion of the procedure and only the first heating element 224 for a second part or portion of the procedure. For example, the clinician may use the first and second heating elements 224, 226 together for a first part of the procedure (e.g., further from the introduction point) and switch to using only the first heating element 224 as the heating elements 224, 226 approach the introduction point. The first plurality of marking segments 236a-f may guide the proximal retraction of the medical device 200 over a first part of the procedure while the second plurality of marking segments 252a-d guide a second part of the procedure. For example, the clinician may position the distal end 206 of the medical device 200 at the distalmost treatment location (relative to the clinician). The clinician may activate the first and second heating elements 224, 226 to treat a first length of vessel. The first and second heating elements 224, 226 may be deactivated in response to a predetermined condition, such as, but not limited to, obtaining a preset temperature, activating for a predetermined length of time, or the like. The clinician may then proximally retract the medical device 200 using the first plurality of marking segments 236a-f to guide withdrawal length. For example, the clinician may proximally retract the medical device 200 by a length of about two consecutive marking segments 236a-f. The clinician may again activate the first and second heating elements 224, 226 to treat a second length of vessel. A proximal end region of the first treated length of vessel may overlap with a distal end region of the second treated length of vessel such that there is a first length of overlap between adjacent treatment regions. The clinician may repeatedly activate the first and second heating elements 224, 226 and proximally retract the medical device 200 using the first plurality of marking segments 236a-f as a guide until the second plurality of marking segments 252a-d become visible (e.g., the medical device 200 has been proximally retracted by a length sufficient to position a proximal most marking segment 252a of the second plurality of marking segments 252a-d exterior to the body). The clinician may then switch to using only the first heating element 224 (e.g., by activating a switch or button at the control and power element 214) and then use the second plurality of marking segments 252a-d to guide the proximal retraction of the medical device. For example, after treatment of a length of the vessel, the clinician may proximally retract the medical device by a length of a single consecutive marking segments 252a-d. The clinician may again activate only the first heating element 224 to treat another length of vessel. A proximal end region of the first treated length of vessel may overlap with a distal end region of the second treated length of vessel such that there is a second length of overlap between adjacent treatment regions. It is contemplated that the second length of overlap may be approximately the same as the first length of overlap. However, this is not required. The second length of overlap may be greater than or less than the first length of overlap depending on a spacing of the second plurality of marking segments 252a-d. While performing the incremental treatments, the clinician may remove the introducer sheath when either the proximal end 244 or the distal end 246 of the sheath removal marking segment 240 is visible depending on whether the clinician is using both the first and second heating elements 224, 226 or only the first heating element 224 when the sheath removal marking segment 240 becomes visible. When the distal final treatment indicator 252d is visible, the clinician may perform a final treatment and remove the medical device 200 from the body. While the current example describes transitioning from the use of both the first and second heating elements 224, 226 to the use of only the first heating element 224 at the transition between the first plurality of marking segments 236a-f to the second plurality of marking segments 252a-d, this is not required. The clinician may transition between the use of both the first and second heating elements 224, 226 to the use of only the first heating element 224 at any point in the procedure. For example, the clinician may use the first plurality of marking segments 236a-f to approximate a withdrawal distance for use of only the first heating element 224 or the clinician may use one or more of the marking segments of the second plurality of marking segments 252a-d to determine a withdrawal distance for the use of both the first and second heating elements 224, 226.

In yet another example, the clinician may choose to use only the first heating element 224 for an entirety of the procedure. The first plurality of marking segments 236a-f may guide the proximal retraction of the medical device 200 over a first part of the procedure while the second plurality of marking segments 252a-d guide a second part of the procedure. For example, the clinician may position the distal end 206 of the medical device 200 at the distalmost treatment location (relative to the clinician). The clinician may activate the first heating element 224 to treat a first length of vessel. The first heating element 224 may be deactivated in response to a predetermined condition, such as, but not limited to, obtaining a preset temperature, activating for a predetermined length of time, or the like. The clinician may then proximally retract the medical device 200 using the first plurality of marking segments 236a-f to guide withdrawal length. For example, the clinician may proximally retract the medical device 220 by a length of about half of one marking segment 236a-f. The clinician may again activate the first heating element 224 to treat a second length of vessel. A proximal end region of the first treated length of vessel may overlap with a distal end region of the second treated length of vessel such that there is a first length of overlap between adjacent treatment regions. It is contemplated that there may be some variability in the overlap regions due to the estimation of proximal retraction distance. The clinician may repeatedly activate the first heating element 224 and proximally retract the medical device 200 using the first plurality of marking segments 236a-f as a guide until the second plurality of marking segments 252a-d become visible (e.g., the medical device 200 has been proximally retracted by a length sufficient to position a proximal most marking segment 252a of the second plurality of marking segments 252a-d exterior to the body). The clinician may then use the second plurality of marking segments 252a-d to guide the proximal retraction of the medical device. For example, after treatment of a length of the vessel, the clinician may proximally retract the medical device by a length of a single marking segment 252a-d. The clinician may again activate the first heating element 224 to treat another length of vessel. A proximal end region of the first treated length of vessel may overlap with a distal end region of the second treated length of vessel such that there is a second length of overlap between adjacent treatment regions. It is contemplated that as the second plurality of marking segments 252a-d are spaced differently than the first plurality of marking segments 236a-f, the second length of overlap may be different than the first length of overlap. In some examples, the second length of overlap may be greater than or less than the first length of overlap. However, this is not required. In some examples, the first length of overlap may be approximately the same as the second length of overlap. While performing the incremental treatments, the clinician may remove the introducer sheath when the distal end 246 of the sheath removal marking segment 240 is visible. When the distal final treatment indicator 252d is visible, the clinician may perform a final treatment and remove the medical device 200 from the body.

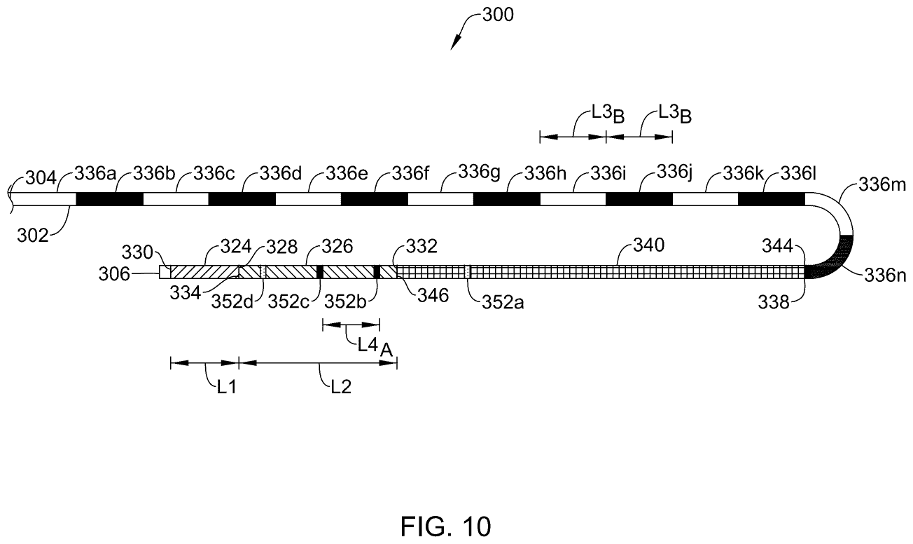

FIG. 10 is a side view of another illustrative medical device 300 configured to perform a thermal ablation procedure. The medical device 300 may be similar in form and function to the medical device 10 described herein with the medical device 300 having an alternative marking scheme. The medical device 300 may be a catheter including a flexible elongate shaft 302. The flexible elongate shaft 302 may extend from a proximal end 304 to a distal end 306. The proximal end 304 of the elongate shaft 302 may include a hub (not explicitly shown) attached thereto for connecting other treatment devices or providing a port (not explicitly shown) for facilitating other treatments. It is contemplated that the stiffness of the elongate shaft 302 may be modified to form a medical device 300 for use in various vessel diameters and various locations within a patient's vasculature. The elongate shaft 302 may further include one or more lumens extending therethrough. For example, the elongate shaft 302 may include a guidewire lumen and/or one or more auxiliary lumens. The lumens may be configured in any way known in the art. For example, the guidewire lumen may extend the entire length of the elongate shaft 302 such as in an over-the-wire catheter or may extend only along a distal portion of the elongate shaft 302 such as in a single operator exchange (SOE) catheter. It is contemplated that if a guidewire is used for access, the guidewire may be proximally retracted prior to treatment unless the guidewire lumen is insulated. These examples are not intended to be limiting, but rather examples of some possible configurations. While not explicitly shown, the medical device 300 may further include temperature sensors/wires, an infusion lumen, radiopaque/echogenic marker bands, fixed guidewire tip, a guidewire lumen, external sheath, and/or other components to facilitate the use and advancement of the medical device 300 within the vasculature.

While not explicitly shown, the medical device 300 may also include an electrical connection or other element similar in form and function to the electrical connection 18 described herein for providing power to a heating element disposed around and/or within the flexible elongate shaft 302. A proximal end of the electrical connection may be connected to a control and power element similar in form and function to the control and power element 22 described herein, which supplies the necessary electrical energy to activate the one or more heating elements 324, 326 at or near a distal end of the electrical connection.

The medical device 300 may further include one or more heating elements or ablation electrodes 324, 326 disposed on and/or surrounding the outer surface of the elongate shaft 302 adjacent the distal end 306. While the medical device 300 is illustrated as including two heating elements 324, 326, it is contemplated that the medical device 300 may include any number of heating elements 324, 326 desired, such as, but not limited to, one, three, four, or more. If multiple heating elements 324, 326 are provided, the heating elements 324, 326 may be longitudinally and/or circumferentially spaced as desired.

In some examples, a first heating element 324 having a first length L1 extending from a proximal end 328 to a distal end 330 may extend proximally from the distal end 306 of the elongate shaft 302. In some embodiments, the distal end 330 of the first heating element 324 may be proximally spaced from the distal end 306 of the elongate shaft 302. In some cases, the distance from the distal end 306 of the elongate shaft 302 to the distal end 330 of the first heating element 324 may be as short as possible so that the treatment starts as distal to the access site as possible without needing to track further while providing a rounded atraumatic tip, insulation at the forward (or distal) facing end of the first heating element 324, and mechanical robustness to the forward (or distal) facing end of the first heating element 324 to be able to push the medical device 300. However, the distance could be made longer, if so desired. A second heating element 326 having a second length L2 extending from a proximal end 332 to a distal end 334 may be positioned proximal to the first heating element 324. The distal end 334 of the second heating element 326 may abut or contact the proximal end 328 of the first heating element 324 such that when the first and second heating elements 324, 326 are activated together a continuous treatment area results. However, this is not required. In some embodiments, there may be a space or gap between the proximal end 328 of the first heating element 324 and the distal end 334 of the second heating element 326. The second length L2 of the second heating element 326 may be greater than the first length L1 of the first heating element 324. However, this is not required. In some cases, the first length L1 of the first heating element 324 may be greater than the second length L2 of the second heating element 326. In yet other examples, the first length L1 of the first heating element 324 and the second length L2 of the second heating element 326 may be approximately the same. In one illustrative example, the first length L1 of the first heating element 324 may be in the range of about 2 cm (0.787 in) to about 4 cm (1.575 in), or about 3 cm (1.181 in) and the second length L2 of the second heating element 326 may be in the range of about 8 cm (3.150 in) to about 10 cm (3.937 in), or about 9 cm (3.543 in). This is just an example. Other heating element lengths may be used, as desired.

In some instances, the heating elements 324, 326 may be circumferential electrodes or elements extending around the outer surface of the elongate shaft 302. A circumferential heating element 324, 326 may allow for circumferential treatment or ablation while reducing and/or eliminating the need for circumferential repositioning of the heating elements 324, 326 and/or elongate shaft 302. In some embodiments, the heating elements 324, 326 may not extend around an entirety of the perimeter of the elongate shaft 302. It is contemplated that multiple heating elements 324, 326 may be circumferentially positioned around the perimeter of the elongate shaft 302 to reduce and/or eliminate the need to circumferentially reposition the elongate shaft 302 to perform 360° ablation.

In some embodiments, the heating elements 324, 326 may be formed of one or more separate structures and attached to the elongate shaft 302. For example, each of the heating elements 324, 326 may be machined or stamped from a monolithic piece of material and subsequently bonded or otherwise attached to the elongate shaft 302. In other embodiments, the heating elements 324, 326 may be formed directly on the surface of the elongate shaft 302. For example, the heating elements 324, 326 may be plated, printed, or otherwise deposited on the surface. In some instances, one or more of the heating elements 324, 326 may be sufficiently radiopaque/echogenic so that it also functions as a visual marker under respective imaging modalities. The heating elements 324, 326 may be formed from any suitable material such as, but not limited to, platinum, gold, stainless steel, cobalt alloys, or other non-oxidizing and/or echogenic materials. In some instances, titanium, tantalum, or tungsten may be used. It is contemplated that the heating elements 324, 326 may take any shape desired, such as, but not limited to, square, rectangular, circular, elliptical, cylindrical, semi-cylindrical, tubular, semi-tubular, helically wound coils (open pitch or closed pitch), bidirectionally wound coils, or the like. In some embodiments, the heating elements 324, 326 may have rounded edges in order to reduce the effects of sharp edges on current density. The size and/or shape of the heating elements 324, 326 may be chosen to optimize the current density without increasing the profile of the medical device 300.

As described above, the first and second heating elements 324, 326 may be electrically coupled to the control and power element using separate electrical connections via the electrical connection. Each electrical connection may extend proximally from a distal end coupled to an individual heating element 324, 326 to a proximal end coupled to the control and power element. In some examples, the electrical connections may extend through the electrical connection or the electrical connection may be formed from the electrical connections. This may allow the first and second heating elements 324, 326 to be activated individually or together. For example, activating only the first heating element 324 may provide a treatment length equal to the first length L1 of the first heating element 324 while activating both the first and the second heating elements 324, 326 may provide a treatment length longer than the first length L1 of the first heating element 324 or equal to the sum of the first length L1 of the first heating element 324 and the second length L2 of the second heating element 326. In other examples, activating only the second heating element 326 may provide yet another treatment length different from the treatment length of the first heating element 324 and the treatment length equal to the sum of the first length L1 of the first heating element 324 and the second length L2 of the second heating element 326.

While not explicitly shown, the medical device 300 may be used with an introducer sheath, which may be similar in form and function to the introducer sheath 40 described herein. In some illustrative examples, the length of the elongate shaft of the introducer sheath may be about 7 cm (2.756 in) or about 11 cm (4.331 in). These are just some examples. The elongate shaft of the introducer sheath may have other lengths, as desired.

To perform a thermal heat treatment of a length of vessel, after preparation of the patient, the introducer sheath may be inserted into the target vessel at an introduction point. Next, the distal end 306 of the medical device 300 may be inserted through the introducer sheath and advanced through the vasculature to a desired treatment location. The medical device 300 may be advanced with or without a guidewire. The entire leg may be repositioned as necessary to facilitate access. When so provided, a guidewire may be removed from the lumen of the medical device prior to treatment if said lumen is not insulated. When the distal end 306 is adjacent to the distalmost end of the target treatment region (relative to the clinician), tumescent anesthesia may be injected into the fascia around the incompetent vein and along the length of an incompetent vein via external, direct injection. The tumescent anesthesia may provide local anesthetic relief as well as pressure along the incompetent vein such that the inner surface of the vein is in closer proximity to the heating elements 324, 326 to optimize heat transfer between the medical device 300 and the vein wall. Next, energy may be supplied to the first and/or second heating element 324, 326. It is contemplated that the clinician may choose to activate the first and second heating elements 324, 326 together to treat a longer length of vessel with each application of energy or the clinician may choose to activate the first heating element 324 individually to treat a smaller length of vessel with each application of energy. As the thermal energy is delivered to the vessel, a cascade of events begin to ultimately occlude the vein. After a predetermined treatment time or a desired temperature is reached, the medical device 300 may be proximally retracted towards the introducer sheath to treat another length of the vessel. The retraction distance may be less than the length L1 of the heating element 324 and/or less than the sum of the lengths L1, L2 of the heating elements 324, 326 used to heat the vessel. This may allow some overlap in the adjacent treatment regions. As will be described in more detail herein, the elongate shaft 302 may include markings configured to help the user withdraw the elongate shaft 302 by a desired amount. The clinician may continue to treat and withdraw the elongate shaft 302 until another marking indicates that the heating element(s) 324, 326 are approaching the introducer sheath. Said marking may indicate to the user that the introducer sheath should be removed or withdrawn to avoid damage to the introducer sheath and layers of dermis, and to allow the treatment energy to penetrate the vessel. After removal of the introducer sheath, the user may continue to activate the first heating element 324 and/or the first and second heating elements 324, 326 and proximally retract or withdraw the elongate shaft 302. This may be repeated until a final treatment marker is visible. At this time, the clinician may perform a final treatment and remove the elongate shaft 302 from the vasculature.

The medical device 300 may further include one or more visual indicia disposed along an outer surface of the elongate shaft 302 and/or heating elements 324, 326. The visual indicia may be positioned along the outer surface of the elongate shaft 302 and/or the heating elements 324, 326 at predefined intervals configured to guide the clinician during use of the medical device 300. In some cases, the predefined intervals may vary along a length of the medical device 300. For example, the medical device 300 may include a first marking scheme along a proximal portion thereof and a second marking scheme along a distal portion thereof. Further, different visual indicia may provide the clinician with different information. For example, the visual indicia may provide the user with reference points to facilitate treating lengths of the vessel, moving the medical device 300, and treating another length of the vessel. In another example, the visual indicia may indicate to the user that the introducer sheath should be removed prior to continuing treatment. In the illustrated embodiment, the elongate shaft 302 may include markings for the use of a single length of introducer sheath. In yet another example, the visual indicia may indicate to the user that a particular treatment should be a final treatment of a given length. Different patterns, colors, and/or other features may be provided for different types of markings. For example, markings used to guide treatment length may have a first type of marking, markings used to guide removal of the sheath may have a second type of marking, and final treatment markings may have a third type of marking. Each of the first, second, and third types of markings may be different from one another or distinguishable from one another. More than three or fewer than three types of markings may be used, as desired. For example, different marking types may be used to guide removal for two or more different lengths of sheaths.

In one illustrative example, the elongate shaft 302 may include a first plurality of markers or marking segments 336a-n extending distally from the proximal end 304 of the elongate shaft 302 to an intermediate location 338 proximal to the distal end 306 of the elongate shaft 302. The markers 336a-n may alternate in color such that each marker 336a-n is easily distinguishable. For example, the elongate shaft 302 may include a first group of markers 336a, 336c, 336c, 336g, 336i, 336k, 336m having a first color and a second group of markers 336b, 336d, 336f, 336h, 336j, 336l 336n having a second color different from the first color where markers of the first color 336a, 336c, 336c, 336g, 336i, 336k, 336m are separated by markers of the second color 336b, 336d, 336f, 336h, 336j, 336l 336n. It is contemplated that the first plurality of marking segments 336a-n may include other distinguishing features such as, but not limited to, patterns, etchings, alphanumeric indicators, or the like. The markers 336a-n may be separate components extending around and coupled to an outer surface of the elongate shaft 302 or may be formed as a single monolithic structure with the elongate shaft 302, as desired. The markers 52a-g may be separate components extending around and coupled to an outer surface of the elongate shaft 302 or may be formed as a single monolithic structure with the elongate shaft 302, as desired. In one example, the first plurality of marking segments 336a-n may be sections of differently colored tubing joined together. Alternatively, or additionally, the first plurality of marking segments 336a-n may be printed onto an outer surface of the elongate shaft 302. The first plurality of marking segments 336a-n may be similar in size and shape to the first plurality of marking segments 136a-m described herein. For example, the first plurality of marking segments 336a-n may each have a third length L3_{B} defined by Equations 5 or 6.

In use, the medical device 300 may be inserted into a vessel through an introducer sheath. The distance the elongate shaft 302 is inserted may depend on the length of the vessel to be treated. Once the elongate shaft 302 is in position, the first and second heating elements 324, 326 may be activated together to treat a first length of the vessel. As the thermal energy is delivered to the vessel, the vessel shrinks and/or collapses on itself to preclude a flow of blood therethrough. Once the first length of the vessel has been treated, the medical device 300 is proximally retracted. The clinician may use the first plurality of marking segments 336a-n and the treatment length to determine how far to retract the medical device 300. For example, the medical device 300 may be retracted until about four adjacent marking segments are removed from the body. The first and second heating elements 324, 326 may again be activated together to treat a second length of the vessel. The distance of proximal retraction of the medical device 300 may be less than the sum of the first and second lengths L1, L2 of the first and second heating elements 324, 326 such that a first treatment overlap length occurs adjacent the distal end 306 of the elongate shaft 302 where a length of the vessel has been treated by both the current treatment and the preceding treatment. Additional treatment overlap lengths may occur for subsequent treatments. The medical device 300 may be repeatedly proximally retracted after each treatment.

After one or more treatments, the proximal end 332 of the second heating element 326 may approach the introducer sheath. The elongate shaft 302 may include one or more sheath removal marking segments 340. In the illustrated embodiment, the elongate shaft 302 may include only a single sheath removal marking segment 340 configured to guide a clinician for removal of an introducer sheath of a single length. For illustrative purposes, the introducer sheath may have an elongate shaft having a length of about 7 cm (2.756 in).

However, this is just an example, the elongate shaft 302 may include markings for introducer sheaths of other lengths. However, the sheath removal marking segment may be proximally or distally displaced on the elongate shaft 302 depending on whether the introducer sheath is longer than 7 cm (2.756 in) or shorter than 7 cm (2.756 in). Generally, the sheath removal marking segment 340 may indicate to the clinician that the introducer sheath should be removed prior to performing any additional treatments to avoid damage to the introducer sheath. It is contemplated that as different length of heating elements 324, 326 and/or different lengths of introducer sheaths may be used, the introducer sheath may be removed at different times in the procedure. For example, an 11 cm (4.331 in) introducer sheath may extend further into the vessel than a 7 cm (2.756 in) introducer sheath and thus the proximal end of the heating elements 324, 326 may approach or near the distal end of an 11 cm (4.331 in) introducer sheath with less proximal retraction of the medical device 300 relative to the 7 cm (2.756 in) introducer sheath. Similarly, the proximal end 332 of the second heating element 326 may approach the distal end region of the introducer sheath with less proximal retraction of the medical device 300 than the proximal end 328 of the first heating element 324.

The sheath removal marking segment 340 may extend from a proximal end 344 to a distal end 346. In some cases, the proximal end 344 of the sheath removal marking segment 340 may be aligned with the intermediate location 338 adjacent to the distal end region of the first plurality of marking segments 336a-n. However, this is not required. The sheath removal marking segment 340 may be sized and shaped to provide a visual indication that prompts the removal of various lengths of introducer sheaths. In one illustrative example, the proximal end 344 of the sheath removal marking segment 340 may indicate to the user that the introducer sheath should be removed when both the first and second heating elements 324, 326 are being used while the distal end 346 of the sheath removal marking segment 340 may indicate to the user that the introducer sheath should be removed when only the first heating element 324 is being used.

The sheath removal marking segment 340 may have a different color, pattern, etc. from the first plurality of marking segments 336a-n to indicate to the user that the marking segment is relaying a different type of information. It is contemplated that the sheath removal marking segment 340 may include other distinguishing features such as, but not limited to, patterns, etchings, alphanumeric indicators, or the like. The sheath removal marking segment 340 may be a separate component extending around and coupled to an outer surface of the elongate shaft 302 or may be formed as a single monolithic structure with the elongate shaft 302, as desired. In one example, the sheath removal marking segment 340 may be a section of differently colored tubing joined together with other portions of the elongate shaft 302. Alternatively, or additionally, the sheath removal marking segment 340 may be printed onto an outer surface of the elongate shaft 302. The sheath removal marking segment 340 may have varying lengths. In one example, the sheath removal marking segments 340 may have a length that is approximately equal to the second length L2 of the second heating element 326. This is just one example. The sheath removal marking segment 340 may have other lengths, as desired.

At or near the intermediate location 338, the elongate shaft 302 may have a change in the marking scheme for the treatment markers. For example, the elongate shaft 302 may include a second plurality of markers or marking segments 352a-d extending distally from the intermediate location 338. The marking segments 352a-d may each have a similar color or marking scheme to one another, although this is not required. The marking segments 352a-d may be separated by regions free from a marking or by other markers, such as, but not limited to the sheath removal marking segment 340. Thus, each marking segment of the second plurality of marking segments 352a-d may have a length that is less than a desired measuring distance. For example, if it is desired to have a marking distance of about 2.5 cm (0.984 in), the length of the marking segments 352a-d may be less than 2.5 cm (0.984 in) to provide separation between each marking segment of the second plurality of marking segments 352a-d. This may help to differentiate the second plurality of marking segments 352a-d from the first plurality of marking segments 336a-n. It is contemplated that the second plurality of marking segments 352a-d may include other distinguishing features such as, but not limited to, colors, patterns, etchings, alphanumeric indicators, or the like. The markers 352a-d may be separate components extending around and coupled to an outer surface of the elongate shaft 302 or may be formed as a single monolithic structure with the elongate shaft 302, as desired. In one example, the second plurality of marking segments 352a-d may be sections of differently colored tubing joined together with other portions of the elongate shaft 302. Alternatively, or additionally, the second plurality of marking segments 352a-d may be printed onto an outer surface of the elongate shaft 302.

It is contemplated that the second plurality of marking segments 352a-d may be spaced from one another by a fourth length L4_{A} that is less than the first length L1 of the first heating element 324. The second plurality of marking segments 352a-d may be similar in form and function to the second plurality of marking segments 68a-g described herein. For example, the fourth length L4_{A} of the space separating the second plurality of marking segments 352a-d may be defined by Equations 3 or 4.

It is contemplated that the second plurality of marking segments 352a-d may also be used to help guide proximal retraction of the medical device 300 when both the first and second heating elements 324, 326 are used together to deliver treatment. In one illustrative example, the first length L1 of the first heating element 324 may be 3 cm (1.181 in), the second length L2 of the second heating element 326 may be 9 cm (3.543 in) such that the treatment length is 12 cm (4.724 in). After the treatment energy has been delivered, the medical device 300 may be retracted by four marking segments of the second plurality of marking segments 352a-d. This may retract the medical device 300 by about 10 cm (3.937 in) (e.g., 2.5 cm multiplied by 4). In this instance, the adjacent treatment regions may overlap by about 2 cm (0.787 in). The number of marking segments of the second plurality of marking segments 352a-d used to guide the proximal retraction when both the first and second heating elements 324, 326 are used may vary depending on the fourth length LA between adjacent marking segments 352a-d.

In some embodiments, one or more of the second plurality of marking segments 352a-d may include a pattern, color, or other feature to distinguish said marking segment from the other marking segments of the second plurality of marking segments 352a-d. In the illustrated embodiment, two marking segments 352a, 352d have a different pattern from the remaining marking segments 352b, 352c. These marking segments 352a, 352d having the different pattern may be final treatment indicators when the introducer sheath has been removed. For example, the final treatment indicators may be positioned such that the clinician is alerted that the heating elements 324, 326 are approaching the incision location and no further application of energy should occur after the current treatment or else unintentional damage to surrounding tissues (e.g., dermis) may occur. It is contemplated that the more proximal final treatment indicator 352a may indicate that if both the first and second heating elements 324, 326 are being used, the current treatment should be the final treatment when the introducer sheath has been removed. The more distal final treatment indicated 352d may indicate that if only the first heating element 324 is being used, the current treatment should be the final treatment when the introducer sheath has been removed. Said differently, once the proximal final treatment indicator 352a is visible a last treatment may be performed using both the first and second heating elements 324, 326 when the introducer sheath has been removed and once the distal final treatment indictor 352d is visible a last treatment may be performed using only the first heating element 324 when the introducer sheath has been removed.

It is contemplated that in some cases, the clinician may choose to use both the first and second heating elements 324, 326 for an entirety of the procedure. The first plurality of marking segments 336a-n may guide the proximal retraction of the medical device 300 over a first part of the procedure while the second plurality of marking segments 352a-d guide a second part of the procedure. For example, the clinician may position the distal end 306 of the medical device 300 at the distalmost treatment location (relative to the clinician). The clinician may activate the first and second heating elements 324, 326 to treat a first length of vessel. The first and second heating elements 324, 326 may be deactivated in response to a predetermined condition, such as, but not limited to, obtaining a preset temperature, activating for a predetermined length of time, or the like. The clinician may then proximally retract the medical device 300 using the first plurality of marking segments 336a-n to guide withdrawal length. For example, the clinician may proximally retract the medical device 300 by a length of about four consecutive marking segments 336a-n. The clinician may again activate the first and second heating elements 324, 326 to treat a second length of vessel. A proximal end region of the first treated length of vessel may overlap with a distal end region of the second treated length of vessel such that there is a first length of overlap between adjacent treatment regions. The clinician may repeatedly activate the first and second heating elements 324, 326 and proximally retract the medical device 300 using the first plurality of marking segments 336a-n as a guide until the second plurality of marking segments 352a-d become visible (e.g., the medical device 300 has been proximally retracted by a length sufficient to position a proximal most marking segment 352a of the second plurality of marking segments 352a-d exterior to the body). The clinician may then use the second plurality of marking segments 352a-d to guide the proximal retraction of the medical device. For example, after treatment of a length of the vessel, the clinician may proximally retract the medical device by a length of about four consecutive marking segments 352a-d. The clinician may again activate the first and second heating elements 324, 326 to treat another length of vessel. A proximal end region of the first treated length of vessel may overlap with a distal end region of the second treated length of vessel such that there is a second length of overlap between adjacent treatment regions. It is contemplated that as the second plurality of marking segments 352a-d may be similarly spaced to the first plurality of marking segments 336a-n, the second length of overlap may be approximately the same as the first length of overlap. However, this is not required. In some examples, the first length of overlap may be greater than or less than the second length of overlap. While performing the incremental treatments, the clinician may remove the introducer sheath when the sheath removal marking segment 340 is visible. The clinician may use the proximal end 344 to determine when to remove the introducer sheath when both the first and second heating elements 324, 326 are being used. When the proximal final treatment indicator 352a is visible, the clinician may perform a final treatment and remove the medical device 300 from the body.

It is contemplated that in some cases, the clinician may choose to use both the first and second heating elements 324, 326 for a first part or portion of the procedure and only the first heating element 324 for a second part or portion of the procedure. For example, the clinician may use the first and second heating elements 324, 326 together for a first part of the procedure (e.g., further from the introduction point) and switch to using only the first heating element 324 as the heating elements 324, 326 approach the introduction point. The first plurality of marking segments 336a-n may guide the proximal retraction of the medical device 300 over a first part of the procedure while the second plurality of marking segments 352a-d guide a second part of the procedure. For example, the clinician may position the distal end 306 of the medical device 300 at the distalmost treatment location (relative to the clinician). The clinician may activate the first and second heating elements 324, 326 to treat a first length of vessel. The first and second heating elements 324, 326 may be deactivated in response to a predetermined condition, such as, but not limited to, obtaining a preset temperature, activating for a predetermined length of time, or the like. The clinician may then proximally retract the medical device 300 using the first plurality of marking segments 336a-n to guide withdrawal length. For example, the clinician may proximally retract the medical device 300 by a length of about four consecutive marking segments 336a-n. The clinician may again activate the first and second heating elements 324, 326 to treat a second length of vessel. A proximal end region of the first treated length of vessel may overlap with a distal end region of the second treated length of vessel such that there is a first length of overlap between adjacent treatment regions. The clinician may repeatedly activate the first and second heating elements 324, 326 and proximally retract the medical device 300 using the first plurality of marking segments 336a-n as a guide until the second plurality of marking segments 352a-d become visible (e.g., the medical device 300 has been proximally retracted by a length sufficient to position a proximal most marking segment 352a of the second plurality of marking segments 352a-d exterior to the body). The clinician may then switch to using only the first heating element 324 (e.g., by activating a switch or button at the control and power element 214) and then use the second plurality of marking segments 352a-d to guide the proximal retraction of the medical device. For example, after treatment of a length of the vessel, the clinician may proximally retract the medical device by a length of a single consecutive marking segments 352a-d. The clinician may again activate only the first heating element 324 to treat another length of vessel. A proximal end region of the first treated length of vessel may overlap with a distal end region of the second treated length of vessel such that there is a second length of overlap between adjacent treatment regions. It is contemplated that the second length of overlap may be less than the first length of overlap. However, this is not required. The second length of overlap may be greater than or approximately equal to the first length of overlap depending on a spacing of the second plurality of marking segments 352a-d. While performing the incremental treatments, the clinician may remove the introducer sheath when either the proximal end 344 or the distal end 346 of the sheath removal marking segment 340 is visible depending on whether the clinician is using both the first and second heating elements 324, 326 or only the first heating element 324 when the sheath removal marking segment 340 becomes visible. When the distal final treatment indicator 352d is visible, the clinician may perform a final treatment and remove the medical device 300 from the body. While the current example describes transitioning from the use of both the first and second heating elements 324, 326 to the use of only the first heating element 324 at the transition between the first plurality of marking segments 336a-n to the second plurality of marking segments 352a-d, this is not required. The clinician may transition between the use of both the first and second heating elements 324, 326 to the use of only the first heating element 324 at any point in the procedure. For example, the clinician may use the first plurality of marking segments 336a-n to approximate a withdrawal distance for use of only the first heating element 324 or the clinician may use one or more of the marking segments of the second plurality of marking segments 352a-d to determine a withdrawal distance for the use of both the first and second heating elements 324, 326.

In yet another example, the clinician may choose to use only the first heating element 324 for an entirety of the procedure. The first plurality of marking segments 336a-n may guide the proximal retraction of the medical device 300 over a first part of the procedure while the second plurality of marking segments 352a-d guide a second part of the procedure. For example, the clinician may position the distal end 306 of the medical device 300 at the distalmost treatment location (relative to the clinician). The clinician may activate the first heating element 324 to treat a first length of vessel. The first heating element 324 may be deactivated in response to a predetermined condition, such as, but not limited to, obtaining a preset temperature, activating for a predetermined length of time, or the like. The clinician may then proximally retract the medical device 300 using the first plurality of marking segments 336a-n to guide withdrawal length. For example, the clinician may proximally retract the medical device 220 by one marking segment 336a-n. The clinician may again activate the first heating element 324 to treat a second length of vessel. A proximal end region of the first treated length of vessel may overlap with a distal end region of the second treated length of vessel such that there is a first length of overlap between adjacent treatment regions. The clinician may repeatedly activate the first heating element 324 and proximally retract the medical device 300 using the first plurality of marking segments 336a-n as a guide until the second plurality of marking segments 352a-d become visible (e.g., the medical device 300 has been proximally retracted by a length sufficient to position a proximal most marking segment 352a of the second plurality of marking segments 352a-d exterior to the body). The clinician may then use the second plurality of marking segments 352a-d to guide the proximal retraction of the medical device. For example, after treatment of a length of the vessel, the clinician may proximally retract the medical device by a length of a single marking segment 352a-d. The clinician may again activate the first heating element 324 to treat another length of vessel. A proximal end region of the first treated length of vessel may overlap with a distal end region of the second treated length of vessel such that there is a second length of overlap between adjacent treatment regions. It is contemplated that as the second plurality of marking segments 352a-d are similarly spaced as the first plurality of marking segments 336a-n, the second length of overlap may be approximately the same as the first length of overlap. However, this is not required. In some examples, the second length of overlap may be greater than or less than the first length of overlap. While performing the incremental treatments, the clinician may remove the introducer sheath when the distal end 346 of the sheath removal marking segment 340 is visible. When the distal final treatment indicator 352d is visible, the clinician may perform a final treatment and remove the medical device 300 from the body.

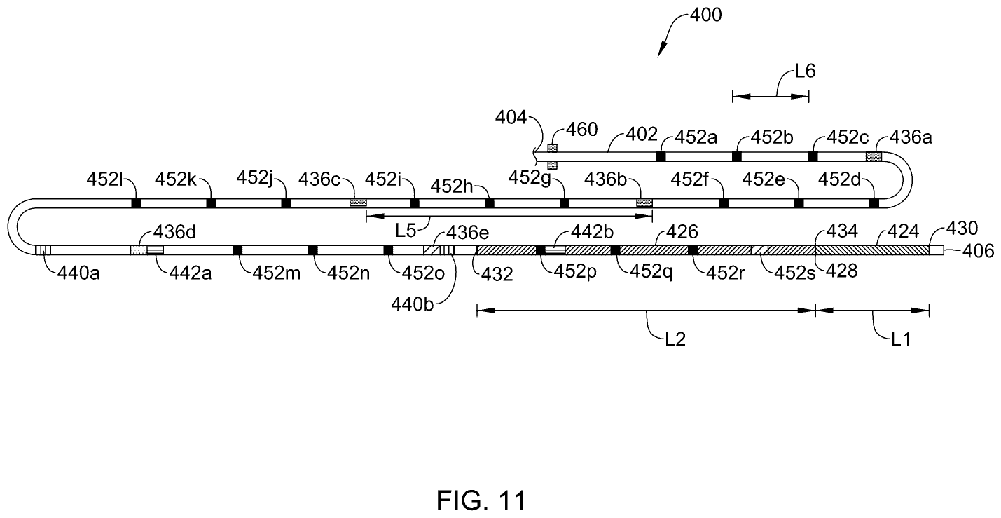

FIG. 11 is a side view of another illustrative medical device 400 configured to perform a thermal ablation procedure. The medical device 400 may be similar in form and function to the medical device 10 described herein with the medical device 400 having an alternative marking scheme. The medical device 400 may be a catheter including a flexible elongate shaft 402. The flexible elongate shaft 402 may extend from a proximal end 404 to a distal end 406. The proximal end 404 of the elongate shaft 402 may include a hub (not explicitly shown) attached thereto for connecting other treatment devices or providing a port (not explicitly shown) for facilitating other treatments. It is contemplated that the stiffness of the elongate shaft 402 may be modified to form a medical device 400 for use in various vessel diameters and various locations within a patient's vasculature. The elongate shaft 402 may further include one or more lumens extending therethrough. For example, the elongate shaft 402 may include a guidewire lumen and/or one or more auxiliary lumens. The lumens may be configured in any way known in the art. For example, the guidewire lumen may extend the entire length of the elongate shaft 402 such as in an over-the-wire catheter or may extend only along a distal portion of the elongate shaft 402 such as in a single operator exchange (SOE) catheter. It is contemplated that if a guidewire is used for access, the guidewire may be proximally retracted prior to treatment unless the guidewire lumen is insulated. These examples are not intended to be limiting, but rather examples of some possible configurations. While not explicitly shown, the medical device 400 may further include temperature sensors/wires, an infusion lumen, radiopaque/echogenic marker bands, fixed guidewire tip, a guidewire lumen, external sheath, and/or other components to facilitate the use and advancement of the medical device 400 within the vasculature.

While not explicitly shown, the medical device 400 may also include an electrical connection or other element similar in form and function to the electrical connection 18 described herein for providing power to a heating element disposed around and/or within the flexible elongate shaft 402. A proximal end of the electrical connection may be connected to a control and power element similar in form and function to the control and power element 22 described herein, which supplies the necessary electrical energy to activate the one or more heating elements 424, 426 at or near a distal end of the electrical connection.

The medical device 400 may further include one or more heating elements or ablation electrodes 424, 426 disposed on and/or surrounding the outer surface of the elongate shaft 402 adjacent the distal end 406. While the medical device 400 is illustrated as including two heating elements 424, 426, it is contemplated that the medical device 400 may include any number of heating elements 424, 426 desired, such as, but not limited to, one, three, four, or more. If multiple heating elements 424, 426 are provided, the heating elements 424, 426 may be longitudinally and/or circumferentially spaced as desired.

In some examples, a first heating element 424 having a first length L1 extending from a proximal end 428 to a distal end 430 may extend proximally from the distal end 406 of the elongate shaft 402. In some embodiments, the distal end 430 of the first heating element 424 may be proximally spaced from the distal end 406 of the elongate shaft 402. In some cases, the distance from the distal end 406 of the elongate shaft 402 to the distal end 430 of the first heating element 424 may be as short as possible so that the treatment starts as distal to the access site as possible without needing to track further while providing a rounded atraumatic tip, insulation at the forward (or distal) facing end of the first heating element 424, and mechanical robustness to the forward (or distal) facing end of the first heating element 424 to be able to push the medical device 400. However, the distance could be made longer, if so desired. A second heating element 426 having a second length L2 extending from a proximal end 432 to a distal end 434 may be positioned proximal to the first heating element 424. The distal end 434 of the second heating element 426 may abut or contact the proximal end 428 of the first heating element 424 such that when the first and second heating elements 424, 426 are activated together a continuous treatment area results. However, this is not required. In some embodiments, there may be a space or gap between the proximal end 428 of the first heating element 424 and the distal end 434 of the second heating element 426. The second length L2 of the second heating element 426 may be greater than the first length L1 of the first heating element 424. However, this is not required. In some cases, the first length L1 of the first heating element 424 may be greater than the second length L2 of the second heating element 426. In yet other examples, the first length L1 of the first heating element 424 and the second length L2 of the second heating element 426 may be approximately the same. In one illustrative example, the first length L1 of the first heating element 424 may be in the range of about 2 cm (0.787 in) to about 4 cm (1.575 in), or about 3 cm (1.181 in) and the second length L2 of the second heating element 426 may be in the range of about 8 cm (3.150 in) to about 10 cm (3.937 in), or about 9 cm (3.543 in). This is just an example. Other heating element lengths may be used, as desired.

In some instances, the heating elements 424, 426 may be circumferential electrodes or elements extending around the outer surface of the elongate shaft 402. A circumferential heating element 424, 426 may allow for circumferential treatment or ablation while reducing and/or eliminating the need for circumferential repositioning of the heating elements 424, 426 and/or elongate shaft 402. In some embodiments, the heating elements 424, 426 may not extend around an entirety of the perimeter of the elongate shaft 402. It is contemplated that multiple heating elements 424, 426 may be circumferentially positioned around the perimeter of the elongate shaft 402 to reduce and/or eliminate the need to circumferentially reposition the elongate shaft 402 to perform 360° ablation.

In some embodiments, the heating elements 424, 426 may be formed of one or more separate structures and attached to the elongate shaft 402. For example, each of the heating elements 424, 426 may be machined or stamped from a monolithic piece of material and subsequently bonded or otherwise attached to the elongate shaft 402. In other embodiments, the heating elements 424, 426 may be formed directly on the surface of the elongate shaft 402. For example, the heating elements 424, 426 may be plated, printed, or otherwise deposited on the surface. In some instances, one or more of the heating elements 424, 426 may be sufficiently radiopaque/echogenic so that it also functions as a visual marker under respective imaging modalities. The heating elements 424, 426 may be formed from any suitable material such as, but not limited to, platinum, gold, stainless steel, cobalt alloys, or other non-oxidizing and/or echogenic materials. In some instances, titanium, tantalum, or tungsten may be used. It is contemplated that the heating elements 424, 426 may take any shape desired, such as, but not limited to, square, rectangular, circular, elliptical, cylindrical, semi-cylindrical, tubular, semi-tubular, helically wound coils (open pitch or closed pitch), bidirectionally wound coils, or the like. In some embodiments, the heating elements 424, 426 may have rounded edges in order to reduce the effects of sharp edges on current density. The size and/or shape of the heating elements 424, 426 may be chosen to optimize the current density without increasing the profile of the medical device 400.

As described above, the first and second heating elements 424, 426 may be electrically coupled to the control and power element using separate electrical connections via the electrical connection. Each electrical connection may extend proximally from a distal end coupled to an individual heating element 424, 426 to a proximal end coupled to the control and power element. In some examples, the electrical connections may extend through the electrical connection or the electrical connection may be formed from the electrical connections. This may allow the first and second heating elements 424, 426 to be activated individually or together. For example, activating only the first heating element 424 may provide a treatment length equal to the first length L1 of the first heating element 424 while activating both the first and the second heating elements 424, 426 may provide a treatment length longer than the first length L1 of the first heating element 424 or equal to the sum of the first length L1 of the first heating element 424 and the second length L2 of the second heating element 426. In other examples, activating only the second heating element 426 may provide yet another treatment length different from the treatment length of the first heating element 424 and the treatment length equal to the sum of the first length L1 of the first heating element 424 and the second length L2 of the second heating element 426.

While not explicitly shown, the medical device 400 may be used with an introducer sheath, which may be similar in form and function to the introducer sheath 40 described herein. In some illustrative examples, the length of the elongate shaft of the introducer sheath may be about 7 cm (2.756 in) or about 11 cm (4.331 in). These are just some examples. The elongate shaft of the introducer sheath may have other lengths, as desired.

To perform a thermal heat treatment of a length of vessel, after preparation of the patient, the introducer sheath may be inserted into the target vessel at an introduction point. A movable marker 460 may be axially displaced along the longitudinal axis of the elongate shaft 402 to indicate the position of the medical device 400 when the first ablation is performed. The movable marker 460 may be moved along a length of the elongate shaft 402 throughout the procedure, as desired, to help guide placement and/or retraction of the elongate shaft 402. It is contemplated that such a movable marker may be provided with any of the medical devices described herein. Next, the distal end 406 of the medical device 400 may be inserted through the introducer sheath and advanced through the vasculature to a desired treatment location. The medical device 400 may be advanced with or without a guidewire. The entire leg may be repositioned as necessary to facilitate access. When so provided, a guidewire may be removed from the lumen of the medical device prior to treatment if said lumen is not insulated. When the distal end 406 is adjacent to the distalmost end of the target treatment region (relative to the clinician), tumescent anesthesia may be injected into the fascia around the incompetent vein and along the length of an incompetent vein via external, direct injection. The tumescent anesthesia may provide local anesthetic relief as well as pressure along the incompetent vein such that the inner surface of the vein is in closer proximity to the heating elements 424, 426 to optimize heat transfer between the medical device 400 and the vein wall. Next, energy may be supplied to the first and/or second heating element 424, 426. It is contemplated that the clinician may choose to activate the first and second heating elements 424, 426 together to treat a longer length of vessel with each application of energy or the clinician may choose to activate the first heating element 424 individually to treat a smaller length of vessel with each application of energy. As the thermal energy is delivered to the vessel, a cascade of events begin to ultimately occlude the vein. After a predetermined treatment time or a desired temperature is reached, the medical device 400 may be proximally retracted toward the introducer sheath to treat another length of the vessel. The retraction distance may be less than the length L1 of the heating element 424 and/or less than the sum of the lengths L1, L2 of the heating elements 424, 426 used to heat the vessel. This may allow some overlap in the adjacent treatment regions. As will be described in more detail herein, the elongate shaft 402 may include markings configured to help the user withdraw the elongate shaft 402 by a desired amount. The clinician may continue to treat and withdraw the elongate shaft 402 until another marking indicates that the heating element(s) 424, 426 are approaching the introducer sheath. Said marking may indicate to the user that the introducer sheath should be removed or withdrawn to avoid damage to the introducer sheath and layers of dermis, and to allow the treatment energy to penetrate the vessel. After removal of the introducer sheath, the user may continue to activate the first heating element 424 and/or the first and second heating elements 424, 426 and proximally retract or withdraw the elongate shaft 402. This may be repeated until a final treatment marker is visible. At this time, the clinician may perform a final treatment and remove the elongate shaft 402 from the vasculature.

The medical device 400 may further include one or more visual indicia disposed along an outer surface of the elongate shaft 402 and/or heating elements 424, 426. The visual indicia may be positioned along the outer surface of the elongate shaft 402 and/or the heating elements 424, 426 at predefined intervals configured to guide the clinician during use of the medical device 400. In some cases, the predefined intervals may vary between marking segments. Further, different visual indicia may provide the clinician with different information. For example, the visual indicia may provide the user with reference points to facilitate treating lengths of the vessel, moving the medical device 400, and treating another length of the vessel. In another example, the visual indicia may indicate to the user that the introducer sheath should be removed prior to continuing treatment. In the illustrated embodiment, the elongate shaft 402 may include markings for the use of two different lengths of introducer sheaths. In yet another example, the visual indicia may indicate to the user that a particular treatment should be a final treatment of a given length. Different patterns, colors, and/or other features may be provided for different types of markings. For example, markings used to guide treatment length may have a first type of marking, markings used to guide removal of the sheath may have a second type of marking, and final treatment markings may have a third type of marking. Each of the first, second, and third types of markings may be different from one another or distinguishable from one another. More than three or fewer than three types of markings may be used, as desired. For example, different marking types may be used to guide removal for two or more different lengths of sheaths.

The elongate shaft 402 may include a first plurality of markers or marking segments 436a-c extending distally from a proximal end region of the elongate shaft 402. A distalmost marking segment 436e may be positioned proximal to the distal end 406 of the elongate shaft 402 and in some cases proximal to the proximal end 432 of the second heating element 426. The marking segments 436a-e may each have a similar color or marking scheme to one another, although this is not required. The marking segments 436a-e may be separated by regions free from a marking or by other markers, such as, but not limited to the sheath removal marking segments 440a, 442a. Thus, each marking segment of the first plurality of marking segments 436a-e may have a length that is less than a desired measuring distance. For example, if it is desired to have a marking distance in the range of about 11-12 cm (4.331-4.724 in), the length of the marking segments 436a-e may be less than 11-12 cm (4.331-4.724 in) to provide separation between each marking segment of the first plurality of marking segments 436a-e. In some cases, the marking segments of the first plurality of marking segments 436a-e may have a length of less than 1 cm (0.394 in). It is contemplated that the first plurality of marking segments 436a-e may include other distinguishing features such as, but not limited to, patterns, etchings, alphanumeric indicators, or the like. The markers 436a-e may be separate components extending around and coupled to an outer surface of the elongate shaft 402 or may be formed as a single monolithic structure with the elongate shaft 402, as desired. In one example, the first plurality of marking segments 436a-e may be sections of differently colored tubing joined together. Alternatively, or additionally, the first plurality of marking segments 436a-e may be printed onto an outer surface of the elongate shaft 402.

It is contemplated that the first plurality of marking segments 436a-e may be spaced from one another by a fifth length L5 that is less than the sum of the first length L1 of the first heating element 424 and the second length L2 of the second heating element 426. In some cases, the fifth length L5 may be measured from a proximal end of a first marking segment 436b to a proximal end of a second adjacent marking segment 436c, the second marking segment 436c distally spaced from first marking segment 436b. This is just one example. In some cases, the fifth length L5 may be measured from distal end to distal end of adjacent marking segments 436a-e. In other examples, the fifth length L5 may be measured from a distal end of a marking segment to the proximal end of the adjacent marking segment. The fifth length L5 may be defined by Equation 7:

$$L 5 < L 1 + L 2 \qquad Equation 7$$

where L5 is the fifth length between each pair of adjacent marking segments of the first plurality of marking segments 436a-e, L1 is the first length of the first heating element 424, and L2 is the second length of the second heating element 426. When the clinician is using both the first and second heating elements 424, 426 together, after a treatment is performed, the elongate shaft 402 may be proximally retracted until a next adjacent marking segment of the first plurality of marking segments 436a-e has been proximally retracted from the body. This may indicate to the clinician that the heating elements 424, 426 have been displaced from the previously treated region by a desired length. In some cases, it may be desirable to provide an overlap in adjacent treatment segments. This may ensure that an entirety of the length of the vessel is treated. The desired overlap may be taken into consideration when determining the distance between adjacent each marking segments of the first plurality of marking segments 436a-e. For example, to obtain a desired overlap in adjacent treatment segments, the fifth length L5 may be defined by Equation 8:

$$L 5 = L 1 + L 2 - OL \qquad Equation 8$$

where L5 is the fifth length between each pair of adjacent marking segments of the first plurality of marking segments 436a-e, L1 is the first length of the first heating element 424, L2 is the second length of the second heating element 426, and OL is a desired treatment overlap length. In one illustrative example, the first length L1 of the first heating element 424 may be 3 cm (1.181 in), the second length L2 of the second heating element 426 may be 9 cm (3.543 in) and the desired overlap between adjacent treatment segments may be 0.5 cm (0.197 in). In this example, the third length L5 of each marking segment of the first plurality of marking segments 52a-g may be 11.5 cm (4.528 in). This is just one example. Thus, when using both the first and second heating elements 424, 426 together, 12 cm (4.724 in) of vessel may be treated in a single application of energy. The medical device 400 may be retracted by about 11.5 cm (4.528 in) before energy is applied again.

The elongate shaft 402 may include a second plurality of markers or marking segments 452a-s extending distally from a proximal end region of the elongate shaft 402. A distalmost marking segment 452s may be positioned proximal to the distal end 406 of the elongate shaft 402 and in some cases proximal to the proximal end 428 of the first heating element 424. The marking segments 452a-s may each have a similar color or marking scheme to one another, although this is not required. The marking segments 452a-s may be separated by regions free from a marking or by other markers, such as, but not limited to the sheath removal marking segments 440a-b, 442a-b. Thus, each marking segment of the second plurality of marking segments 452a-s may have a length that is less than a desired measuring distance. For example, if it is desired to have a marking distance in the range of about 2-3 cm (0.787-1.181 in), the length of the marking segments 452a-s may be less than 2-3 cm (0.787-1.181 in) to provide separation between each marking segment of the second plurality of marking segments 452a-s. In some cases, the marking segments of the second plurality of marking segments 452a-s may have a length of less than 1 cm (0.394 in). It is contemplated that the second plurality of marking segments 452a-s may include other distinguishing features such as, but not limited to, patterns, etchings, alphanumeric indicators, or the like. It is contemplated that the second plurality of marking segments 452a-s may be visually distinct from the first plurality of marking segments 436a-c. The markers 452a-s may be separate components extending around and coupled to an outer surface of the elongate shaft 402 or may be formed as a single monolithic structure with the elongate shaft 402, as desired. In one example, the second plurality of marking segments 452a-s may be sections of differently colored tubing joined together with other portions of the elongate shaft 402. Alternatively, or additionally, the second plurality of marking segments 452a-s may be printed onto an outer surface of the elongate shaft 402.

It is contemplated that the second plurality of marking segments 452a-s may be spaced from one another by a sixth length L6 that is less than the first length L1 of the first heating element 424. In some cases, the sixth length L6 may be measured from a proximal end of a first marking segment 452b to a proximal end of a second adjacent marking segment 452c, the second marking segment 452c distally spaced from first marking segment 452b. This is just one example. In some cases, the sixth length L6 may be measured from distal end to distal end of adjacent marking segments 452a-s. In other examples, the sixth length L6 may be measured from a distal end of a marking segment to the proximal end of the adjacent marking segment. The sixth length L6 may be defined by Equation 9:

$$L 6 < L 1 \qquad Equation 9$$

where L6 is the sixth length between each pair of adjacent marking segments of the second plurality of marking segments 452a-s and L1 is the first length of the first heating element 424. When a marking segment of the first plurality of marking segments 436a-e is disposed between adjacent marking segments of the second plurality of marking segments 452a-s, said marking segment of the first plurality of marking segments 436a-e may function as one of the marking segments of the second plurality of marking segments 452a-s. For example, marking segment 436a is disposed between marking segment 452c and 452d. Marking segment 436a may be disposed a length L6 from marking segment 452c and marking segment 452d may be disposed a length L6 from marking segment 436a. Said differently, the marking segments of the first plurality of marking segments 436a-e may be considered to also be included in the second plurality of marking segments 452a-s.

When the clinician is using only the first heating element 424, after a treatment is performed, the elongate shaft 402 may be proximally retracted until a next adjacent marking segment of the second plurality of marking segments 452a-s has been proximally retracted from the body. This may indicate to the clinician that the heating element 424 has been displaced from the previously treated region by a desired length. In some cases, it may be desirable to provide an overlap in adjacent treatment segments. This may ensure that an entirety of the length of the vessel is treated. The desired overlap may be taken into consideration when determining the distance between adjacent each marking segments of the second plurality of marking segments 452a-s. For example, to obtain a desired overlap in adjacent treatment segments, the sixth length L6 may be defined by Equation 10:

$$L 6 = L 1 - O L \qquad Equation 10$$

where L6 is the sixth length between each pair of adjacent marking segments of the second plurality of marking segments 452a-s and L1 is the first length of the first heating element 424, and OL is a desired treatment overlap length. In one illustrative example, the first length L1 of the first heating element 424 may be 3 cm (1.181 in) the desired overlap between adjacent treatment segments may be 0.5 cm (0.197 in). In this example, the sixth length L6 between adjacent marking segments of the second plurality of marking segments 452a-s may be 2.5 cm (0.984 in). This is just one example. Thus, when using only the first heating element 424, 3 cm (1.181 in) of vessel may be treated in a single application of energy. The medical device 400 may be retracted by about 2.5 cm before energy is applied again.

It is contemplated that having two distinguishable marking systems extending along the length of the elongate shaft 402, or substantial portions thereof, the clinician may easily switch between using both the first and second heating elements 424, 426 or using only the first heating element 424 at any time during the procedure. Further having each marking system 436a-c, 452a-s extending along the length of the elongate shaft 402 or substantial portions thereof, may allow the clinician to consistently use only the first heating element 424 or both the first and second heating elements 424, 426 for an entire procedure.

In use, the medical device 400 may be inserted into a vessel through an introducer sheath. The distance the elongate shaft 402 is inserted may depend on the length of the vessel to be treated. Once the elongate shaft 402 is in position, the first and second heating elements 424, 426 may be activated together or the first heating element 424 may be activated individually to treat a first length of the vessel. As the thermal energy is delivered to the vessel, the vessel shrinks and/or collapses on itself to preclude a flow of blood therethrough. Once the first length of the vessel has been treated, the medical device 400 is proximally retracted. The clinician may use the first plurality of marking segments 436a-e to determine and the treatment length how far to retract the medical device 400 if both the first and the second heating elements 424, 426 are being activated. For example, the medical device 400 may be retracted until about a next marking segment of the first plurality of marking segments 436a-e is removed from the body. The first and second heating elements 424, 426 may again be activated together to treat a second length of the vessel. The distance of proximal retraction of the medical device 400 may be less than the sum of the first and second lengths L1, L2 of the first and second heating elements 424, 426 such that a first treatment overlap length occurs adjacent the distal end 406 of the elongate shaft 402 where a length of the vessel has been treated by both the current treatment and the preceding treatment. Additional treatment overlap lengths may occur for subsequent treatments. The medical device 400 may be repeatedly proximally retracted after each treatment.

The clinician may use the second plurality of marking segments 452a-s (as well as the first plurality of marking segments 436a-e) and the treatment length to determine how far to retract the medical device 400 if only the first heating element 424 is being activated. For example, the medical device 400 may be retracted until about a next marking segment of the second plurality of marking segments 452a-s or the next marking segment of the first plurality of marking segments 436a-e is removed from the body. The first heating element 424 may again be activated to treat a second length of the vessel. The distance of proximal retraction of the medical device 400 may be less than the first length L1 of the first heating element 424 such that a first treatment overlap length occurs adjacent the distal end 406 of the elongate shaft 402 where a length of the vessel has been treated by both the current treatment and the preceding treatment. Additional treatment overlap lengths may occur for subsequent treatments. The medical device 400 may be repeatedly proximally retracted after each treatment.

After one or more treatments, the proximal end 432 of the second heating element 426 may approach the introducer sheath. The elongate shaft 402 may include one or more sheath removal marking segments 440a-b, 442a-b. In the illustrated embodiment, the elongate shaft 402 may include four sheath removal marking segment 440a-b, 442a-b configured to guide a clinician for removal of an introducer sheath having one of two lengths and two treatment lengths. For example, a first sheath removal marking segment 440a may provide an indication to the clinician that an 11 cm (4.331 in) introducer sheath should be removed when using the first and second heating elements 424, 426 together. A second sheath removal marking segment 442a positioned distal to the first sheath removal marking segment 440a may provide an indication to the clinician that a 7 cm (2.756 in) introducer sheath should be removed when using the first and second heating elements 424, 426 together. A third sheath removal marking segment 440b positioned distal to the second sheath removal marking segment 442a may provide an indication to the clinician that an 11 cm (4.331 in) introducer sheath should be removed when using only first heating element 424. A fourth sheath removal marking segment 442b positioned distal to the third sheath removal marking segment 440b may provide an indication to the clinician that a 7 cm (2.756 in) introducer sheath should be removed when using only first heating element 424. These are just examples. It is contemplated that the number and/or spacing of the sheath removal marking segments 440a-b, 442a-b may be determined by the length of the first and/or second heating elements 424, 426 and/or the length of the available introducer sheaths.

The sheath removal marking segments 440a-b, 442a-b may have a different color, pattern, etc. from the first plurality of marking segments 436a-e, the second plurality of marking segments 452a-s and/or each other to indicate to the user that the marking segment is relaying a different type of information. It is contemplated that the sheath removal marking segments 440a-b, 442a-b may include other distinguishing features such as, but not limited to, patterns, etchings, alphanumeric indicators, or the like. The sheath removal marking segments 440a-b, 442a-b may be separate components extending around and coupled to an outer surface of the elongate shaft 402 or may be formed as a single monolithic structure with the elongate shaft 402, as desired. In one example, the sheath removal marking segments 440a-b, 442a-b may be sections of differently colored tubing joined together with other portions of the elongate shaft 402. Alternatively, or additionally, sheath removal marking segments 440a-b, 442a-b may be printed onto an outer surface of the elongate shaft 402. The sheath removal marking segments 440a-b, 442a-b may have varying lengths.

In some embodiments, the distalmost marking segment 436e of the first plurality of marking segments 436a-e may include a pattern, color, or other feature to distinguish said marking segment 436e from the other marking segments of the first plurality of marking segments 436a-c. This marking segment 436e having the different pattern may be a final treatment indicator when both the first and second heating elements 424, 426 arc being used and the introducer sheath has been removed. Similarly, the distalmost marking segment 452s of the second plurality of marking segments 452a-s may include a pattern, color, or other feature to distinguish said marking segment 452s from the other marking segments of the second plurality of marking segments 452a-s-e. This marking segments 452s having the different pattern may be a final treatment indicator when only the first heating element 424 is being used and the introducer sheath has been removed. For example, the final treatment indicator 436e may be positioned such that the clinician is alerted that the heating elements 424, 426 are approaching the incision location and no further application of energy should occur after the current treatment or else unintentional damage to surrounding tissues (e.g., dermis) may occur and the final treatment indicator 452s may be positioned such that the clinician is alerted that the first heating elements 424 is are approaching the incision location and no further application of energy should occur after the current treatment or else unintentional damage to surrounding tissues (e.g., dermis) may occur.

It is contemplated that in some cases, the clinician may choose to use both the first and second heating elements 424, 426 for an entirety of the procedure. The first plurality of marking segments 436a-e may guide the proximal retraction of the medical device 400 over an entirety of the procedure. For example, the clinician may position the distal end 406 of the medical device 400 at the distalmost treatment location (relative to the clinician). The clinician may activate the first and second heating elements 424, 426 to treat a first length of vessel. The first and second heating elements 424, 426 may be deactivated in response to a predetermined condition, such as, but not limited to, obtaining a preset temperature, activating for a predetermined length of time, or the like. The clinician may then proximally retract the medical device 400 using the first plurality of marking segments 436a-e to guide withdrawal length. For example, the clinician may proximally retract the medical device 400 until the next marking segments 436a-e of the first plurality of marking segments 436a-e is visible. The clinician may again activate the first and second heating elements 424, 426 to treat a second length of vessel. A proximal end region of the first treated length of vessel may overlap with a distal end region of the second treated length of vessel such that there is a first length of overlap between adjacent treatment regions. The clinician may repeatedly activate the first and second heating elements 424, 426 and proximally retract the medical device 400 using the first plurality of marking segments 436a-e as a guide until the distalmost marking segment 436c of the first plurality of marking segments 436a-e becomes visible indicating that the current treatment should be the final treatment While performing the incremental treatments, the clinician may remove the introducer sheath when either the first or second sheath removal marking segments 440a, 442a are visible (depending on the length of the introducer sheath). When the final treatment indicator 436e is visible, the clinician may perform a final treatment and remove the medical device 400 from the body.

It is contemplated that in some cases, the clinician may choose to use both the first and second heating elements 424, 426 for a first part or portion of the procedure and only the first heating element 424 for a second part or portion of the procedure. For example, the clinician may use the first and second heating elements 424, 426 together for a first part of the procedure (e.g., further from the introduction point) and switch to using only the first heating element 424 as the heating elements 424, 426 approach the introduction point. The first plurality of marking segments 436a-e may guide the proximal retraction of the medical device 400 over a first part of the procedure while the second plurality of marking segments 452a-s (and possibly some of the first plurality of marking segments 436a-e) guide a second part of the procedure. For example, the clinician may position the distal end 406 of the medical device 400 at the distalmost treatment location (relative to the clinician). The clinician may activate the first and second heating elements 424, 426 to treat a first length of vessel. The first and second heating elements 424, 426 may be deactivated in response to a predetermined condition, such as, but not limited to, obtaining a preset temperature, activating for a predetermined length of time, or the like. The clinician may then proximally retract the medical device 400 using the first plurality of marking segments 436a-e to guide withdrawal length. For example, the clinician may proximally retract the medical device 400 until the next marking segments 436a-e of the first plurality of marking segments 436a-e is visible. The clinician may again activate the first and second heating elements 424, 426 to treat a second length of vessel. A proximal end region of the first treated length of vessel may overlap with a distal end region of the second treated length of vessel such that there is a first length of overlap between adjacent treatment regions. The clinician may repeatedly activate the first and second heating elements 424, 426 and proximally retract the medical device 400 using the first plurality of marking segments 436a-e as a guide until the clinician switches to using only the first heating element 424. The clinician may then switch to using the second plurality of marking segments 452a-s and any intermediary marking segments of the first plurality of marking segments 432a-e to guide the proximal retraction of the medical device. For example, after treatment of a length of the vessel, the clinician may proximally retract the medical device by a length of a single consecutive marking segments 452a-s, 436a-c. The clinician may again activate only the first heating element 424 to treat another length of vessel. A proximal end region of the first treated length of vessel may overlap with a distal end region of the second treated length of vessel such that there is a second length of overlap between adjacent treatment regions. It is contemplated that the second length of overlap may be approximately the same as the first length of overlap. However, this is not required. The second length of overlap may be greater than or less than the first length of overlap depending on a spacing of the second plurality of marking segments 452a-s and/or a spacing of the first plurality of marking segments 436a-e. While performing the incremental treatments, the clinician may remove the introducer sheath when any one of the sheath removal marking segment 440a-b, 442b is visible. The clinician may select the appropriate sheath removal marking segment 440a-b, 442a-b based on whether the clinician is using both the first and second heating elements 424, 426 or only the first heating element 424 and a length of the introducer sheath when the sheath removal marking segment 440a-b, 442a-b becomes visible. When the final treatment indicator 452s is visible, the clinician may perform a final treatment and remove the medical device 400 from the body. The clinician may transition between the use of both the first and second heating elements 424, 426 to the use of only the first heating element 424 at any point in the procedure.

In yet another example, the clinician may choose to use only the first heating element 424 for an entirety of the procedure. The second plurality of marking segments 452a-s as well as the first plurality of marking segments 436a-e may guide the proximal retraction of the medical device 400 over an entirety of the procedure. For example, the clinician may position the distal end 406 of the medical device 400 at the distalmost treatment location (relative to the clinician). The clinician may activate the first heating element 424 to treat a first length of vessel. The first heating element 424 may be deactivated in response to a predetermined condition, such as, but not limited to, obtaining a preset temperature, activating for a predetermined length of time, or the like. The clinician may then proximally retract the medical device 400 using the next marking segment of cither the first plurality of marking segments 436a-e or the second plurality of marking segments 452a-s to guide withdrawal length. For example, the clinician may proximally retract the medical device 400 by one marking segment of either the first plurality of marking segments 436a-e or the second plurality of marking segments 452a-s depending on which is visible next. The clinician may again activate the first heating element 424 to treat a second length of vessel. A proximal end region of the first treated length of vessel may overlap with a distal end region of the second treated length of vessel such that there is a first length of overlap between adjacent treatment regions. The clinician may repeatedly activate the first heating element 424 and proximally retract the medical device 400 using the first plurality of marking segments 436a-e or the second plurality of marking segments 452a-s as a guide until the final treatment marking segments 452s become visible. While performing the incremental treatments, the clinician may remove the introducer sheath when the third or fourth sheath removal marking segment 440b, 442b is visible (depending on the length of the introducer sheath). When the distal final treatment indicator 452s is visible, the clinician may perform a final treatment and remove the medical device 400 from the body.

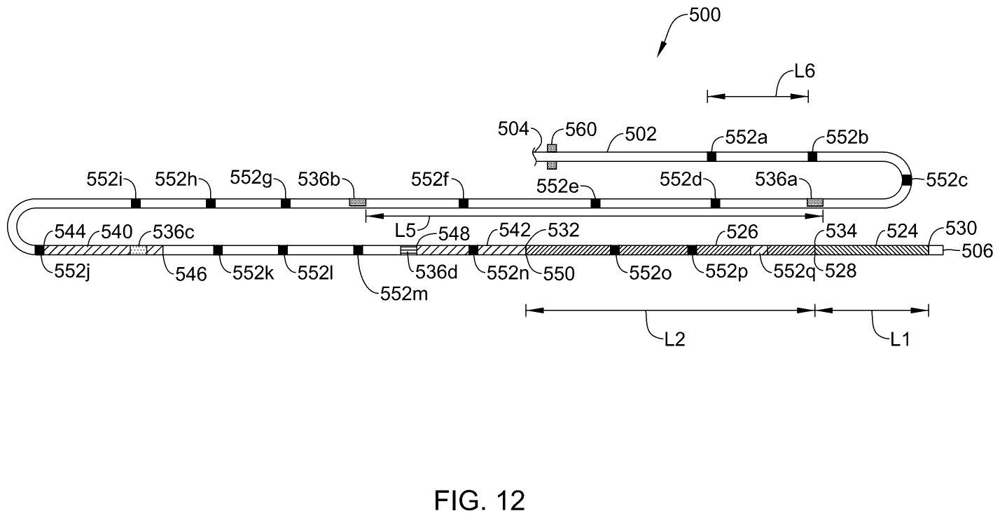

FIG. 12 is a side view of another illustrative medical device 500 configured to perform a thermal ablation procedure. The medical device 500 may be similar in form and function to the medical device 10 described herein with the medical device 500 having an alternative marking scheme. The medical device 500 may be a catheter including a flexible elongate shaft 502. The flexible elongate shaft 502 may extend from a proximal end 504 to a distal end 506. The proximal end 504 of the elongate shaft 502 may include a hub (not explicitly shown) attached thereto for connecting other treatment devices or providing a port (not explicitly shown) for facilitating other treatments. It is contemplated that the stiffness of the elongate shaft 502 may be modified to form a medical device 500 for use in various vessel diameters and various locations within a patient's vasculature. The elongate shaft 502 may further include one or more lumens extending therethrough. For example, the elongate shaft 502 may include a guidewire lumen and/or one or more auxiliary lumens. The lumens may be configured in any way known in the art. For example, the guidewire lumen may extend the entire length of the elongate shaft 502 such as in an over-the-wire catheter or may extend only along a distal portion of the elongate shaft 502 such as in a single operator exchange (SOE) catheter. It is contemplated that if a guidewire is used for access, the guidewire may be proximally retracted prior to treatment unless the guidewire lumen is insulated. These examples are not intended to be limiting, but rather examples of some possible configurations. While not explicitly shown, the medical device 500 may further include temperature sensors/wires, an infusion lumen, radiopaque/echogenic marker bands, fixed guidewire tip, a guidewire lumen, external sheath, and/or other components to facilitate the use and advancement of the medical device 500 within the vasculature.

While not explicitly shown, the medical device 500 may also include an electrical connection or other element similar in form and function to the electrical connection 18 described herein for providing power to a heating element disposed around and/or within the flexible elongate shaft 502. A proximal end of the electrical connection may be connected to a control and power element similar in form and function to the control and power element 22 described herein, which supplies the necessary electrical energy to activate the one or more heating elements 524, 526 at or near a distal end of the electrical connection.

The medical device 500 may further include one or more heating elements or ablation electrodes 524, 526 disposed on and/or surrounding the outer surface of the elongate shaft 502 adjacent the distal end 506. While the medical device 500 is illustrated as including two heating elements 524, 526, it is contemplated that the medical device 500 may include any number of heating elements 524, 526 desired, such as, but not limited to, one, three, four, or more. If multiple heating elements 524, 526 are provided, the heating elements 524, 526 may be longitudinally and/or circumferentially spaced as desired.

In some examples, a first heating element 524 having a first length L1 extending from a proximal end 528 to a distal end 530 may extend proximally from the distal end 506 of the elongate shaft 502. In some embodiments, the distal end 530 of the first heating element 524 may be proximally spaced from the distal end 506 of the elongate shaft 502. In some cases, the distance from the distal end 506 of the elongate shaft 502 to the distal end 530 of the first heating element 524 may be as short as possible so that the treatment starts as distal to the access site as possible without needing to track further while providing a rounded atraumatic tip, insulation at the forward (or distal) facing end of the first heating element 524, and mechanical robustness to the forward (or distal) facing end of the first heating element 524 to be able to push the medical device 500. However, the distance could be made longer, if so desired. A second heating element 526 having a second length L2 extending from a proximal end 532 to a distal end 534 may be positioned proximal to the first heating element 524. The distal end 534 of the second heating element 526 may abut or contact the proximal end 528 of the first heating element 524 such that when the first and second heating elements 524, 526 are activated together a continuous treatment area results. However, this is not required. In some embodiments, there may be a space or gap between the proximal end 528 of the first heating element 524 and the distal end 534 of the second heating element 526. The second length L2 of the second heating element 526 may be greater than the first length L1 of the first heating element 524. However, this is not required. In some cases, the first length L1 of the first heating element 524 may be greater than the second length L2 of the second heating element 526. In yet other examples, the first length L1 of the first heating element 524 and the second length L2 of the second heating element 526 may be approximately the same. In one illustrative example, the first length L1 of the first heating element 524 may be in the range of about 2 cm (0.787 in) to about 4 cm (1.575 in), or about 3 cm (1.181 in) and the second length L2 of the second heating element 526 may be in the range of about 8 cm (3.150 in) to about 10 cm (3.937 in), or about 9 cm (3.543 in). This is just an example. Other heating element lengths may be used, as desired.

In some instances, the heating elements 524, 526 may be circumferential electrodes or elements extending around the outer surface of the elongate shaft 502. A circumferential heating element 524, 526 may allow for circumferential treatment or ablation while reducing and/or eliminating the need for circumferential repositioning of the heating elements 524, 526 and/or elongate shaft 502. In some embodiments, the heating elements 524, 526 may not extend around an entirety of the perimeter of the elongate shaft 502. It is contemplated that multiple heating elements 524, 526 may be circumferentially positioned around the perimeter of the elongate shaft 502 to reduce and/or eliminate the need to circumferentially reposition the elongate shaft 502 to perform 360° ablation.

In some embodiments, the heating elements 524, 526 may be formed of one or more separate structures and attached to the elongate shaft 502. For example, each of the heating elements 524, 526 may be machined or stamped from a monolithic piece of material and subsequently bonded or otherwise attached to the elongate shaft 502. In other embodiments, the heating elements 524, 526 may be formed directly on the surface of the elongate shaft 502. For example, the heating elements 524, 526 may be plated, printed, or otherwise deposited on the surface. In some instances, one or more of the heating elements 524, 526 may be sufficiently radiopaque/echogenic so that it also functions as a visual marker under respective imaging modalities. The heating elements 524, 526 may be formed from any suitable material such as, but not limited to, platinum, gold, stainless steel, cobalt alloys, or other non-oxidizing and/or echogenic materials. In some instances, titanium, tantalum, or tungsten may be used. It is contemplated that the heating elements 524, 526 may take any shape desired, such as, but not limited to, square, rectangular, circular, elliptical, cylindrical, semi-cylindrical, tubular, semi-tubular, helically wound coils (open pitch or closed pitch), bidirectionally wound coils, or the like. In some embodiments, the heating elements 524, 526 may have rounded edges in order to reduce the effects of sharp edges on current density. The size and/or shape of the heating elements 524, 526 may be chosen to optimize the current density without increasing the profile of the medical device 500.

As described above, the first and second heating elements 524, 526 may be electrically coupled to the control and power element using separate electrical connections via the electrical connection. Each electrical connection may extend proximally from a distal end coupled to an individual heating element 524, 526 to a proximal end coupled to the control and power element. In some examples, the electrical connections may extend through the electrical connection or the electrical connection may be formed from the electrical connections. This may allow the first and second heating elements 524, 526 to be activated individually or together. For example, activating only the first heating element 524 may provide a treatment length equal to the first length L1 of the first heating element 524 while activating both the first and the second heating elements 524, 526 may provide a treatment length longer than the first length L1 of the first heating element 524 or equal to the sum of the first length L1 of the first heating element 524 and the second length L2 of the second heating element 526. In other examples, activating only the second heating element 526 may provide yet another treatment length different from the treatment length of the first heating element 524 and the treatment length equal to the sum of the first length L1 of the first heating element 524 and the second length L2 of the second heating element 526.

While not explicitly shown, the medical device 500 may be used with an introducer sheath, which may be similar in form and function to the introducer sheath 40 described herein. In some illustrative examples, the length of the elongate shaft of the introducer sheath may be about 7 cm (2.756 in) or about 11 cm (4.331 in). These are just some examples. The elongate shaft of the introducer sheath may have other lengths, as desired.

To perform a thermal heat treatment of a length of vessel, after preparation of the patient, the introducer sheath may be inserted into the target vessel at an introduction point. A movable marker 560 may be axially displaced along the longitudinal axis of the elongate shaft 502 to indicate the position of the medical device 500 when the first ablation is performed. The movable marker 560 may be moved along a length of the elongate shaft 502 throughout the procedure, as desired, to help guide placement and/or retraction of the elongate shaft 502. Next, the distal end 506 of the medical device 500 may be inserted through the introducer sheath and advanced through the vasculature to a desired treatment location. The medical device 500 may be advanced with or without a guidewire. The entire leg may be repositioned as necessary to facilitate access. When so provided, a guidewire may be removed from the lumen of the medical device prior to treatment if said lumen is not insulated. When the distal end 506 is adjacent to the distalmost end of the target treatment region (relative to the clinician), tumescent anesthesia may be injected into the fascia around the incompetent vein and along the length of an incompetent vein via external, direct injection. The tumescent anesthesia may provide local anesthetic relief as well as pressure along the incompetent vein such that the inner surface of the vein is in closer proximity to the heating elements 524, 526 to optimize heat transfer between the medical device 500 and the vein wall. Next, energy may be supplied to the first and/or second heating element 524, 526. It is contemplated that the clinician may choose to activate the first and second heating elements 524, 526 together to treat a longer length of vessel with each application of energy or the clinician may choose to activate the first heating element 524 individually to treat a smaller length of vessel with each application of energy. As the thermal energy is delivered to the vessel, a cascade of events begin to ultimately occlude the vein. After a predetermined treatment time or a desired temperature is reached, the medical device 500 may be proximally retracted toward the introducer sheath to treat another length of the vessel. The retraction distance may be less than the length L1 of the heating element 524 and/or less than the sum of the lengths L1, L2 of the heating elements 524, 526 used to heat the vessel. This may allow some overlap in the adjacent treatment regions. As will be described in more detail herein, the elongate shaft 502 may include markings configured to help the user withdraw the elongate shaft 502 by a desired amount. The clinician may continue to treat and withdraw the elongate shaft 502 until another marking indicates that the heating element(s) 524, 526 are approaching the introducer sheath. Said marking may indicate to the user that the introducer sheath should be removed or withdrawn to avoid damage to the introducer sheath and layers of dermis and to allow the treatment energy to penetrate the vessel. After removal of the introducer sheath, the user may continue to activate the first heating element 524 and/or the first and second heating elements 524, 526 and proximally retract or withdraw the elongate shaft 502. This may be repeated until a final treatment marker is visible. At this time, the clinician may perform a final treatment and remove the elongate shaft 502 from the vasculature.

The medical device 500 may further include one or more visual indicia disposed along an outer surface of the elongate shaft 502 and/or heating elements 524, 526. The visual indicia may be positioned along the outer surface of the elongate shaft 502 and/or the heating elements 524, 526 at predefined intervals configured to guide the clinician during use of the medical device 500. In some cases, the predefined intervals may vary between marking segments. Further, different visual indicia may provide the clinician with different information. For example, the visual indicia may provide the user with reference points to facilitate treating lengths of the vessel, moving the medical device 500, and treating another length of the vessel. In another example, the visual indicia may indicate to the user that the introducer sheath should be removed prior to continuing treatment. In the illustrated embodiment, the elongate shaft 502 may include markings for the use of two different lengths of introducer sheaths. In yet another example, the visual indicia may indicate to the user that a particular treatment should be a final treatment of a given length. Different patterns, colors, and/or other features may be provided for different types of markings. For example, markings used to guide treatment length may have a first type of marking, markings used to guide removal of the sheath may have a second type of marking, and final treatment markings may have a third type of marking. Each of the first, second, and third types of markings may be different from one another or distinguishable from one another. More than three or fewer than three types of markings may be used, as desired. For example, different marking types may be used to guide removal for two or more different lengths of sheaths.

The elongate shaft 502 may include a first plurality of markers or marking segments 536a-d extending distally from a proximal end region of the elongate shaft 502. A distalmost marking segment 536d may be positioned proximal to the distal end 506 of the elongate shaft 502 and in some cases proximal to the proximal end 532 of the second heating element 526. The marking segments 536a-d may each have a similar color or marking scheme to one another, although this is not required. The marking segments 536a-d may be separated by regions free from a marking or by other markers, such as, but not limited to the sheath removal marking segments 540, 542. Thus, each marking segment of the first plurality of marking segments 536a-d may have a length that is less than a desired measuring distance. For example, if it is desired to have a marking distance in the range of about 11-12 cm (4.331-4.724 in), the length of the marking segments 536a-d may be less than 11-12 cm (4.331-4.724 in) to provide separation between each marking segment of the first plurality of marking segments 536a-d. In some cases, the marking segments of the first plurality of marking segments 536a-d may have a length of less than 1 cm (0.394 in). It is contemplated that the first plurality of marking segments 536a-d may include other distinguishing features such as, but not limited to, patterns, etchings, alphanumeric indicators, or the like. The markers 536a-d may be separate components extending around and coupled to an outer surface of the elongate shaft 502 or may be formed as a single monolithic structure with the elongate shaft 502, as desired. The markers 52a-g may be separate components extending around and coupled to an outer surface of the elongate shaft 502 or may be formed as a single monolithic structure with the elongate shaft 502, as desired. In one example, the first plurality of marking segments 536a-d may be sections of differently colored tubing joined together. Alternatively, or additionally, the first plurality of marking segments 536a-d may be printed onto an outer surface of the elongate shaft 502.

It is contemplated that the first plurality of marking segments 536a-d may be spaced from one another by a fifth length L5 that is less than the sum of the first length L1 of the first heating element 524 and the second length L2 of the second heating element 526. In some cases, the fifth length L5 may be measured from a proximal end of a first marking segment 536a to a proximal end of a second adjacent marking segment 536b, the second marking segment 536b distally spaced from first marking segment 536a. This is just one example. In some cases, the fifth length L5 may be measured from distal end to distal end of adjacent marking segments 536a-d. In other examples, the fifth length L5 may be measured from a distal end of a marking segment to the proximal end of the adjacent marking segment. The fifth length L5 may be similar to the fifth length L5 described with respect to FIG. 11. For example, the fifth length may be defined by Equations 7 or 8. When the clinician is using both the first and second heating elements 524, 526 together, after a treatment is performed, the elongate shaft 502 may be proximally retracted until a next adjacent marking segment of the first plurality of marking segments 536a-d has been proximally retracted from the body. This may indicate to the clinician that the heating elements 524, 526 have been displaced from the previously treated region by a desired length. In some cases, it may be desirable to provide an overlap in adjacent treatment segments. This may ensure that an entirety of the length of the vessel is treated. The desired overlap may be taken into consideration when determining the distance between adjacent each marking segments of the first plurality of marking segments 536a-d. In one illustrative example, the first length L1 of the first heating element 524 may be 3 cm (1.181 in), the second length L2 of the second heating element 526 may be 9 cm (3.543 in) and the desired overlap between adjacent treatment segments may be 0.5 cm (0.197 in). In this example, the third length L5 of each marking segment of the first plurality of marking segments 52a-g may be 11.5 cm (4.528 in). This is just one example. Thus, when using both the first and second heating elements 524, 526 together, 12 cm (4.724 in) of vessel may be treated in a single application of energy. The medical device 500 may be retracted by about 11.5 cm (4.528 in) before energy is applied again.

The elongate shaft 502 may include a second plurality of markers or marking segments 552a-q extending distally from a proximal end region of the elongate shaft 502. A distalmost marking segment 552q may be positioned proximal to the distal end 506 of the elongate shaft 502 and in some cases proximal to the proximal end 528 of the first heating element 524. The marking segments 552a-q may each have a similar color or marking scheme to one another, although this is not required. The marking segments 552a-q may be separated by regions free from a marking or by other markers, such as, but not limited to the sheath removal marking segments 540, 542. Thus, each marking segment of the second plurality of marking segments 552a-q may have a length that is less than a desired measuring distance. For example, if it is desired to have a marking distance in the range of about 2-3 cm (0.787-1.181 in), the length of the marking segments 552a-q may be less than 2-3 cm (0.787-1.181 in) to provide separation between each marking segment of the second plurality of marking segments 552a-q. In some cases, the marking segments of the second plurality of marking segments 552a-q may have a length of less than 1 cm (0.394 in). It is contemplated that the second plurality of marking segments 552a-q may include other distinguishing features such as, but not limited to, patterns, etchings, alphanumeric indicators, or the like. It is contemplated that the second plurality of marking segments 552a-q may be visually distinct from the first plurality of marking segments 536a-d. The markers 552a-q may be separate components extending around and coupled to an outer surface of the elongate shaft 502 or may be formed as a single monolithic structure with the elongate shaft 502, as desired. In one example, the second plurality of marking segments 552a-q may be sections of differently colored tubing joined together with other portions of the elongate shaft 502. Alternatively, or additionally, the second plurality of marking segments 552a-q may be printed onto an outer surface of the elongate shaft 502.

It is contemplated that the second plurality of marking segments 552a-q may be spaced from one another by a sixth length L6 that is less than the first length L1 of the first heating element 524. In some cases, the sixth length L6 may be measured from a proximal end of a first marking segment 552a to a proximal end of a second adjacent marking segment 552b, the second marking segment 552b distally spaced from first marking segment 552a. This is just one example. In some cases, the sixth length L6 may be measured from distal end to distal end of adjacent marking segments 552a-q. In other examples, the sixth length L6 may be measured from a distal end of a marking segment to the proximal end of the adjacent marking segment. The sixth length L6 may be similar to the sixth length L6 described with respect to FIG. 11. For example, the sixth length L6 may be defined by Equations 9 or 10. When a marking segment of the first plurality of marking segments 536a-d is disposed between adjacent marking segments of the second plurality of marking segments 552a-q, said marking segment of the first plurality of marking segments 536a-d may function as one of the marking segments of the second plurality of marking segments 552a-q. For example, marking segment 536a is disposed between marking segment 552c and 552d. Marking segment 536a may be disposed a length L6 from marking segment 552c and marking segment 552d may be disposed a length L6 from marking segment 536a. Said differently, the marking segments of the first plurality of marking segments 536a-d may be considered to also be included in the second plurality of marking segments 552a-q.

When the clinician is using only the first heating element 524, after a treatment is performed, the elongate shaft 502 may be proximally retracted until a next adjacent marking segment of the second plurality of marking segments 552a-q has been proximally retracted from the body. This may indicate to the clinician that the heating element 524 has been displaced from the previously treated region by a desired length. In some cases, it may be desirable to provide an overlap in adjacent treatment segments. This may ensure that an entirety of the length of the vessel is treated. The desired overlap may be taken into consideration when determining the distance between adjacent each marking segments of the second plurality of marking segments 552a-q. In one illustrative example, the first length L1 of the first heating element 524 may be 3 cm (1.181 in) the desired overlap between adjacent treatment segments may be 0.5 cm (0.197 in). In this example, the sixth length L6 between adjacent marking segments of the second plurality of marking segments 552a-q may be 2.5 cm (0.984 in). This is just one example. Thus, when using only the first heating element 524, 3 cm (1.181 in) of vessel may be treated in a single application of energy. The medical device 500 may be retracted by about 2.5 cm (0.984 in) before energy is applied again.

It is contemplated that having two distinguishable marking systems extending along the length of the elongate shaft 502, or substantial portions thereof, the clinician may easily switch between using both the first and second heating elements 524, 526 or using only the first heating element 524 at any time during the procedure. Further having each marking system 536a-d, 552a-q extending along the length of the elongate shaft 502, or substantial portions thereof, may allow the clinician to consistently use only the first heating element 524 or both the first and second heating elements 524, 526 for an entire procedure.

In use, the medical device 500 may be inserted into a vessel through an introducer sheath. The distance the elongate shaft 502 is inserted may depend on the length of the vessel to be treated. Once the elongate shaft 502 is in position, the first and second heating elements 524, 526 may be activated together or the first heating element 524 may be activated individually to treat a first length of the vessel. As the thermal energy is delivered to the vessel, the vessel shrinks and/or collapses on itself to preclude a flow of blood therethrough. Once the first length of the vessel has been treated, the medical device 500 is proximally retracted. The clinician may use the first plurality of marking segments 536a-d and the treatment length to determine how far to retract the medical device 500 if both the first and the second heating elements 524, 526 are being activated. For example, the medical device 500 may be retracted until about a next marking segment of the first plurality of marking segments 536a-d is removed from the body. The first and second heating elements 524, 526 may again be activated together to treat a second length of the vessel. The distance of proximal retraction of the medical device 500 may be less than the sum of the first and second lengths L1, L2 of the first and second heating elements 524, 526 such that a first treatment overlap length occurs adjacent the distal end 506 of the elongate shaft 502 where a length of the vessel has been treated by both the current treatment and the preceding treatment. Additional treatment overlap lengths may occur for subsequent treatments. The medical device 500 may be repeatedly proximally retracted after each treatment.

The clinician may use the second plurality of marking segments 552a-q (as well as the first plurality of marking segments 536a-d) and the treatment length to determine how far to retract the medical device 500 if only the first heating element 524 is being activated. For example, the medical device 500 may be retracted until about a next marking segment of the second plurality of marking segments 552a-q or the next marking segment of the first plurality of marking segments 536a-d is removed from the body. The first heating element 524 may again be activated to treat a second length of the vessel. The distance of proximal retraction of the medical device 500 may be less than the first length L1 of the first heating element 524 such that a first treatment overlap length occurs adjacent the distal end 506 of the elongate shaft 502 where a length of the vessel has been treated by both the current treatment and the preceding treatment. Additional treatment overlap lengths may occur for subsequent treatments. The medical device 500 may be repeatedly proximally retracted after each treatment.

After one or more treatments, the proximal end 532 of the second heating element 526 may approach the introducer sheath. The elongate shaft 502 may include one or more sheath removal marking segments such as, but not limited to, a proximal sheath removal marking segment 540 and a distal sheath removal marking segment 542 distally spaced from the proximal sheath removal marking segment 540. Generally, the proximal and distal sheath removal marking segments 540, 542 may indicate to the clinician that the introducer sheath should be removed prior to performing any additional treatments to avoid damage to the introducer sheath. It is contemplated that as different lengths of heating elements 524, 526 and/or different lengths of introducer sheaths may be used, the introducer sheath may be removed at different times in the procedure. For example, an 11 cm (4.331 in) introducer sheath may extend further into the vessel than a 7 cm (2.756 in) introducer sheath and thus the proximal end of the heating elements 524, 526 may approach or near the distal end of an 11 cm (4.331 in) introducer sheath with less proximal retraction of the medical device 500 relative to the 7 cm (2.756 in) introducer sheath. Similarly, the proximal end 532 of the second heating element 526 may approach the distal end region of the introducer sheath with less proximal retraction of the medical device 500 than the proximal end 528 of the first heating element 524.

Generally, the proximal sheath removal marking segment 540 may be configured to indicate to the clinician that the introducer sheath should be removed when both the first and second heating elements 524, 526 are being activated together. The proximal sheath removal marking segment 540 may extend from a proximal end 544 to a distal end 546. The proximal sheath removal marking segment 540 may be sized and shaped to provide a visual indication that prompts the removal of various lengths of introducer sheaths. In one illustrative example, the proximal end 544 of the proximal sheath removal marking segment 540 may indicate to the user that the introducer sheath should be removed when an 11 cm (4.331 in) introducer sheath is being used while the distal end 546 of the proximal sheath removal marking segment 540 may indicate to the user that the introducer sheath should be removed when a 7 cm (2.756 in) introducer sheath is being used.

Generally, the distal sheath removal marking segment 542 may be configured to indicate to the clinician that the introducer sheath should be removed when only the first heating element 524 is being activated. When only the first heating element 524 is being activated, the introducer sheath may remain in the body for more treatments than if the first and second heating elements 524, 526 are used together. The distal sheath removal marking segment 542 may be positioned distal to the proximal sheath removal marking segment 540. The distal sheath removal marking segment 542 may extend from a proximal end 548 to a distal end 550. In some cases, the proximal end 548 of the distal sheath removal marking segment 542 may be longitudinally spaced from the proximal end 544 of the proximal sheath removal marking segment 540 by a length approximately equal to the length L2 of the second heating element 526. However, this is not required. The spacing may be less than or greater than L2, as desired. The distal sheath removal marking segment 542 may be sized and shaped to provide a visual indication that prompts the removal of various lengths of introducer sheaths. In one illustrative example, the proximal end 548 of the distal sheath removal marking segment 542 may indicate to the user that the introducer sheath should be removed when an 11 cm (4.331 in) introducer sheath is being used while the distal end 550 of the distal sheath removal marking segment 542 may indicate to the user that the introducer sheath should be removed when a 7 cm (2.756 in) introducer sheath is being used.

The proximal and/or distal sheath removal marking segments 540, 542 may have a different color, pattern, etc. from the first plurality of marking segments 536a-d and/or the second plurality of marking segments 552a-q to indicate to the user that the marking segment is relaying a different type of information. It is contemplated that the proximal and/or distal sheath removal marking segments 540, 542 may include other distinguishing features such as, but not limited to, patterns, etchings, alphanumeric indicators, or the like. The proximal and/or distal sheath removal marking segments 540, 542 may be separate components extending around and coupled to an outer surface of the elongate shaft 502 or may be formed as a single monolithic structure with the elongate shaft 502, as desired. In one example, the proximal and/or distal sheath removal marking segments 540, 542 may be sections of differently colored tubing joined together with other portions of the elongate shaft 502. Alternatively, or additionally, the proximal and/or distal sheath removal marking segments 540, 542 may be printed onto an outer surface of the elongate shaft 502. The proximal and/or distal sheath removal marking segments 540, 542 may have varying lengths. In one example, the proximal and/or distal sheath removal marking segments 540, 542 may each have a length that is approximately equal to the difference in lengths of the available introducer sheaths. For example, if the available introducer sheaths have lengths of 11 cm (4.331 in) and 7 cm (2.756 in), the proximal and/or distal sheath removal marking segments 540, 542 may each have a length of approximately 4 cm (1.575 in). This is just one example. The proximal and/or distal sheath removal marking segments 540, 542 may have lengths longer than 4 cm (1.575 in) or shorter than 4 cm (1.575 in), as desired. Further, the proximal and/or distal sheath removal marking segments 540, 542 need not have the same length.

In some embodiments, the distalmost marking segment 536d of the first plurality of marking segments 536a-d may include a pattern, color, or other feature to distinguish said marking segment 536d from the other marking segments of the first plurality of marking segments 536a-d. This marking segment 536d having the different pattern may be a final treatment indicator when both the first and second heating elements 524, 526 are being used and the introducer sheath has been removed. Similarly, the distalmost marking segment 552q of the second plurality of marking segments 552a-q may include a pattern, color, or other feature to distinguish said marking segment 552q from the other marking segments of the second plurality of marking segments 552a-q-c. This marking segment 552q having the different pattern may be a final treatment indicator when only the first heating element 524 is being used and the introducer sheath has been removed. For example, the final treatment indicator 536d may be positioned such that the clinician is alerted that the heating elements 524, 526 are approaching the incision location and no further application of energy should occur after the current treatment or else unintentional damage to surrounding tissues (e.g., dermis) may occur and the final treatment indicator 552q may be positioned such that the clinician is alerted that the first heating elements 524 is are approaching the incision location and no further application of energy should occur after the current treatment or else unintentional damage to surrounding tissues (e.g., dermis) may occur.

It is contemplated that in some cases, the clinician may choose to use both the first and second heating elements 524, 526 for an entirety of the procedure. The first plurality of marking segments 536a-d may guide the proximal retraction of the medical device 500 over an entirety of the procedure. For example, the clinician may position the distal end 506 of the medical device 500 at the distalmost treatment location (relative to the clinician). The clinician may activate the first and second heating elements 524, 526 to treat a first length of vessel. The first and second heating elements 524, 526 may be deactivated in response to a predetermined condition, such as, but not limited to, obtaining a preset temperature, activating for a predetermined length of time, or the like. The clinician may then proximally retract the medical device 500 using the first plurality of marking segments 536a-d to guide withdrawal length. For example, the clinician may proximally retract the medical device 500 until the next marking segments 536a-d of the first plurality of marking segments 536a-d is visible. The clinician may again activate the first and second heating elements 524, 526 to treat a second length of vessel. A proximal end region of the first treated length of vessel may overlap with a distal end region of the second treated length of vessel such that there is a first length of overlap between adjacent treatment regions. The clinician may repeatedly activate the first and second heating elements 524, 526 and proximally retract the medical device 500 using the first plurality of marking segments 536a-d as a guide until the distalmost marking segment 536d of the first plurality of marking segments 536a-d becomes visible indicating that the current treatment should be the final treatment While performing the incremental treatments, the clinician may remove the introducer sheath when the proximal sheath removal marking segment 540 is visible. The clinician may use the proximal end 544 or the distal end 546 to determine when to remove the introducer sheath based on the length of the introducer sheath used with the procedure. When the final treatment indicator 536d is visible, the clinician may perform a final treatment and remove the medical device 500 from the body.

It is contemplated that in some cases, the clinician may choose to use both the first and second heating elements 524, 526 for a first part or portion of the procedure and only the first heating element 524 for a second part or portion of the procedure. For example, the clinician may use the first and second heating elements 524, 526 together for a first part of the procedure (e.g., further from the introduction point) and switch to using only the first heating element 524 as the heating elements 524, 526 approach the introduction point. The first plurality of marking segments 536a-d may guide the proximal retraction of the medical device 500 over a first part of the procedure while the second plurality of marking segments 552a-q (and possibly some of the first plurality of marking segments 536a-d) guide a second part of the procedure. For example, the clinician may position the distal end 506 of the medical device 500 at the distalmost treatment location (relative to the clinician). The clinician may activate the first and second heating elements 524, 526 to treat a first length of vessel. The first and second heating elements 524, 526 may be deactivated in response to a predetermined condition, such as, but not limited to, obtaining a preset temperature, activating for a predetermined length of time, or the like. The clinician may then proximally retract the medical device 500 using the first plurality of marking segments 536a-d to guide withdrawal length. For example, the clinician may proximally retract the medical device 500 until the next marking segments 536a-d of the first plurality of marking segments 536a-d is visible. The clinician may again activate the first and second heating elements 524, 526 to treat a second length of vessel. A proximal end region of the first treated length of vessel may overlap with a distal end region of the second treated length of vessel such that there is a first length of overlap between adjacent treatment regions. The clinician may repeatedly activate the first and second heating elements 524, 526 and proximally retract the medical device 500 using the first plurality of marking segments 536a-d as a guide until the clinician switches to using only the first heating element 524. The clinician may then switch to using the second plurality of marking segments 552a-q and any intermediary marking segments of the first plurality of marking segments 532a-e to guide the proximal retraction of the medical device. For example, after treatment of a length of the vessel, the clinician may proximally retract the medical device by a length of a single consecutive marking segments 552a-q, 536a-d. The clinician may again activate only the first heating element 524 to treat another length of vessel. A proximal end region of the first treated length of vessel may overlap with a distal end region of the second treated length of vessel such that there is a second length of overlap between adjacent treatment regions. It is contemplated that the second length of overlap may be approximately the same as the first length of overlap. However, this is not required. The second length of overlap may be greater than or less than the first length of overlap depending on a spacing of the second plurality of marking segments 552a-q and/or a spacing of the first plurality of marking segments 536a-d. While performing the incremental treatments, the clinician may remove the introducer sheath when either the proximal sheath removal marking segment 540 or the distal sheath removal marking segment 542 is visible depending on whether the clinician is using both the first and second heating elements 524, 526 or only the first heating element 524 when the proximal and/or distal sheath removal marking segments 540, 542 become visible. The clinician may use the proximal ends 544, 548 or the distal ends 546, 550 to determine when to remove the introducer sheath based on the length of the introducer sheath used with the procedure. When the final treatment indicator 552q is visible, the clinician may perform a final treatment and remove the medical device 500 from the body. The clinician may transition between the use of both the first and second heating elements 524, 526 to the use of only the first heating element 524 at any point in the procedure.

In yet another example, the clinician may choose to use only the first heating element 524 for an entirety of the procedure. The second plurality of marking segments 552a-q as well as the first plurality of marking segments 536a-d may guide the proximal retraction of the medical device 500 over an entirety of the procedure. For example, the clinician may position the distal end 506 of the medical device 500 at the distalmost treatment location (relative to the clinician). The clinician may activate the first heating element 524 to treat a first length of vessel. The first heating element 524 may be deactivated in response to a predetermined condition, such as, but not limited to, obtaining a preset temperature, activating for a predetermined length of time, or the like. The clinician may then proximally retract the medical device 500 using the next marking segment of either the first plurality of marking segments 536a-d or the second plurality of marking segments 552a-q to guide withdrawal length. For example, the clinician may proximally retract the medical device 500 by one marking segment of either the first plurality of marking segments 536a-d or the second plurality of marking segments 552a-q depending on which is visible next. The clinician may again activate the first heating element 524 to treat a second length of vessel. A proximal end region of the first treated length of vessel may overlap with a distal end region of the second treated length of vessel such that there is a first length of overlap between adjacent treatment regions. The clinician may repeatedly activate the first heating element 524 and proximally retract the medical device 500 using the first plurality of marking segments 536a-d or the second plurality of marking segments 552a-q as a guide until the final treatment marking segments 552q become visible. While performing the incremental treatments, the clinician may remove the introducer sheath when the distal sheath removal marking segment 542 is visible. The clinician may use the proximal end 548 or the distal end 550 to determine when to remove the introducer sheath based on the length of the introducer sheath used with the procedure. When the distal final treatment indicator 552q is visible, the clinician may perform a final treatment and remove the medical device 500 from the body.

It is contemplated that in any of the embodiments described herein, treatment may be performed more than once at a given location prior to proximally retracting the medical device.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A medical device, comprising:
a flexible elongate shaft extending from a proximal end to a distal end;
a first heating element having a first length (L1) positioned adjacent to the distal end of the flexible elongate shaft;
a second heating element having a second length (L2) different from the first length, the second heating element positioned proximal to and abutting a proximal end of the first heating element;
a first plurality of marking segments extending from a proximal end of the flexible elongate shaft to an intermediate region positioned between the proximal end and the distal end of the flexible elongate shaft, each marking segment of the first plurality of marking segments having a third length, wherein the third length satisfies the following equation:
$$L 3 < \frac{L 1 + L 2}{2}$$
where L3 is the third length, L1 is the first length and L2 is the second length;
a second plurality of marking segments extending distally from a location distal to the first plurality of marking segments, each marking segment of the second plurality of marking segments spaced from one another by a fourth length, wherein the fourth length satisfies the following equation:
$$L 4 < L 1$$
where L4 is the fourth length;
wherein the first heating element is configured to operate alone or in combination with the second heating element.

2. The medical device of claim 1 further comprising a first sheath removal marking segment, the first sheath removal marking segment disposed between the first plurality of marking segments and the second plurality of marking segments.

3. The medical device of claim 2, further comprising a second sheath removal marking segment, the second sheath removal marking segment positioned proximal to and abutting a proximal end of the second heating element.

4. The medical device of claim 1, further comprising a first final treatment indicator, the first final treatment indicator providing an indication of a final treatment using the first heating element in combination with the second heating element.

5. The medical device of claim 4, further comprising a second final treatment indicator, the second final treatment indicator providing an indication of a final treatment using the first heating element.

6. The medical device of claim 5, wherein the second final treatment indicator is distal to the first final treatment indicator.

7. The medical device of claim 1, wherein the first length of the first heating element is less than the second length of the second heating element.

8. The medical device of claim 1, wherein the third length satisfies the following equation:
$$L 3 = \frac{( ( L 1 + L 2 ) - OL )}{2}$$
where OL is a treatment overlap length.

9. The medical device of claim 1, wherein the fourth length satisfies the following equation:
$$L 4 = L 1 - O L$$
where OL is a treatment overlap length.

10. The medical device of claim 1, wherein the third length satisfies the following equation:
$$L 3 = L 1 - O L$$
where OL is a treatment overlap length.

11. A medical device, comprising:
a flexible elongate shaft extending from a proximal end to a distal end;
a first heating element having a first length (L1) positioned adjacent to the distal end of the flexible elongate shaft;
a second heating element having a second length (L2) different from the first length, the second heating element positioned proximal to and abutting a proximal end of the first heating element;
a first plurality of marking segments extending from a proximal end of the flexible elongate shaft to an intermediate region positioned between the proximal end and the distal end of the flexible elongate shaft, each marking segment of the first plurality of marking segments having a third length (L3), the third length less than the first length of the first heating element; and
a second plurality of marking segments extending distally from a location distal to the first plurality of marking segments, each marking segment of the second plurality of marking segments spaced from one another by a fourth length (LA), the fourth length less than the first length of the first heating element;
wherein the first heating element is configured to operate alone or in combination with the second heating element.

12. The medical device of claim 11, further comprising a first sheath removal marking segment, the first sheath removal marking segment disposed between the first plurality of marking segments and the second plurality of marking segments.

13. The medical device of claim 12, further comprising a second sheath removal marking segment, the second sheath removal marking segment positioned proximal to and abutting a proximal end of the second heating element.

14. The medical device of claim 11, further comprising a first final treatment indicator, the first final treatment indicator providing an indication of a final treatment using the first heating element in combination with the second heating element.

15. The medical device of claim 14, further comprising a second final treatment indicator, the second final treatment indicator providing an indication of a final treatment using the first heating element.

16. The medical device of claim 15, wherein the second final treatment indicator is distal to the first final treatment indicator.

17. The medical device of claim 11 wherein the first length of the first heating element is less than the second length of the second heating element.

18. The medical device of claim 11, wherein the third length satisfies the following equation:
$$L 3 = L 1 - O L$$
where L3 is the third length, L1 is the first length, and OL is a treatment overlap length.

19. The medical device of claim 11, wherein the fourth length satisfies the following equation:
$$L 4 = L 1 - O L$$
where L4 is the fourth length, L1 is the first length, and OL is a treatment overlap length.

20. A medical device, comprising:
a flexible elongate shaft extending from a proximal end to a distal end;
a first heating element having a first length positioned adjacent to the distal end of the flexible elongate shaft;
a second heating element having a second length different from the first length, the second heating element positioned proximal to and abutting a proximal end of the first heating element;
a first plurality of marking segments extending from a proximal end of the flexible elongate shaft to an intermediate region positioned between the proximal end and the distal end of the flexible elongate shaft, each marking segment of the first plurality of marking segments having a third length; and
a second plurality of marking segments extending distally from a location distal to the first plurality of marking segments, each marking segment of the second plurality of marking segments spaced from one another by a fourth length, the fourth length less than the first length of the first heating element;
wherein the first heating element is configured to operate alone or in combination with the second heating element.
